(19) United States
(12) Patent Application Publication
(10) Pub. No.: US 2023/0320130 A1
(43) Pub. Date: Oct. 5, 2023

(54) LIGHT-EMITTING DEVICE, LIGHT-EMITTING APPARATUS, DISPLAY APPARATUS, ELECTRONIC DEVICE, AND LIGHTING DEVICE

(71) Applicant: Semiconductor Energy Laboratory Co., Ltd., Atsugi-shi, Kanagawa-ken (JP)

(72) Inventors: Takeyoshi WATABE, Atsugi (JP); Airi UEDA, Zama (JP); Hiromitsu KIDO, Atsugi (JP); Nobuharu OHSAWA, Zama (JP); Satoshi SEO, Sagamihara (JP)

(21) Appl. No.: 18/017,520

(22) PCT Filed: Jul. 28, 2021
(86) PCT No.: PCT/IB2021/056834

§ 371 Date: Jan. 23, 2023

(30) Foreign Application Priority Data
Aug. 11, 2020 (JP) 2020-135814

(51) Int. Cl. H10K 50/858; H10K 50/15; C07C 211/61; C09K 11/06

(52) U.S. Cl. CPC H10K 50/858; H10K 50/156; C07C 211/61; C09K 11/06; H10K 2102/351

(57) ABSTRACT

A novel light-emitting device that is highly convenient, useful, or reliable is provided. The light-emitting device includes a function of emitting light, a first electrode, a second electrode, and a unit. The light has the maximum peak at a wavelength (lambda). The second electrode includes a region overlapping with the first electrode. The unit includes a region positioned between the first electrode and the second electrode. The unit includes a first layer, a second layer, and a third layer. The first layer includes a region positioned between the second layer and the third layer. The first layer contains a light-emitting material. The second layer includes a fourth layer and a fifth layer. The fifth layer includes a region positioned between the fourth layer and the first layer. The fourth layer contains a first organic compound. The first organic compound has a first refractive index with respect to light having the wavelength (lambda). The fifth layer is in contact with the fourth layer. The fifth layer contains a second organic compound. The second organic compound has a second refractive index with respect to light having the wavelength (lambda). The second refractive index is lower than the first refractive index.

### TECHNICAL FIELD

One embodiment of the present invention relates to a light-emitting device, a light-emitting apparatus, a display apparatus, an electronic device, or a lighting device.

Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Thus, more specifically, examples of the technical field of one embodiment of the present invention disclosed in this specification include a semiconductor device, a display apparatus, a light-emitting apparatus, a power storage device, a memory device, a driving method thereof, and a manufacturing method thereof.

### BACKGROUND ART

Light-emitting devices (organic EL devices) utilizing electroluminescence (EL) using organic compounds have been put to more practical use. In the basic structure of such light-emitting devices, an organic compound layer containing a light-emitting material (an EL layer) is sandwiched between a pair of electrodes. Carriers (holes and electrons) are injected by application of voltage to the element, and recombination energy of the carriers is used, whereby light emission can be obtained from the light-emitting material.

Such light-emitting devices are of self-light-emitting type and thus have advantages over liquid crystal, such as high visibility and no need for backlight when used in pixels of a display, and are suitable as flat panel display elements. Displays including such light-emitting devices are also highly advantageous in that they can be thin and lightweight. Moreover, such light-emitting devices also have a feature that the response speed is extremely fast.

Since light-emitting layers of such light-emitting devices can be successively formed two-dimensionally, planar light emission can be achieved. This feature is difficult to realize with point light sources typified by incandescent lamps and LEDs or linear light sources typified by fluorescent lamps; thus, the light-emitting devices also have great potential as planar light sources, which can be applied to lighting and the like.

Displays or lighting devices using light-emitting devices can be suitably used for a variety of electronic devices as described above, and research and development of light-emitting devices have progressed for more favorable characteristics.

One of the problems often discussed in talking about an organic EL element is outcoupling efficiency being low. In particular, the attenuation due to reflection caused by a difference in refractive index between adjacent layers is a main cause of a reduction in element efficiency. In order to reduce this effect, a structure in which a layer formed of a low refractive index material is formed inside an EL layer has been proposed (see Patent Document 1, for example).

Although a light-emitting device having this structure can have higher outcoupling efficiency and higher external quantum efficiency than a light-emitting device having a conventional structure, it is not easy to form such a layer with a low refractive index inside an EL layer without adversely affecting other critical characteristics of the light-emitting device. This is because a low refractive index and a high carrier-transport property or reliability when the material is used for a light-emitting device have a trade-off relation. This problem is caused because the carrier-transport property or reliability of an organic compound largely depends on an unsaturated bond, and an organic compound having many unsaturated bonds tends to have a high refractive index.

### REFERENCE

### Patent Document

[Patent Document 1] United States Patent Application Publication No. 2020/0176692

### SUMMARY OF THE INVENTION

### Problems to be Solved by the Invention

An object of one embodiment of the present invention is to provide a novel light-emitting device that is highly convenient, useful, or reliable. Another object is to provide a novel light-emitting apparatus that is highly convenient, useful, or reliable. Another object is to provide a novel display apparatus that is highly convenient, useful, or reliable. Another object is to provide a novel electronic device that is highly convenient, useful, or reliable. Another object is to provide a novel lighting device that is highly convenient, useful, or reliable. Another object is to provide a novel light-emitting device, a novel light-emitting apparatus, a novel display apparatus, a novel electronic device, or a novel lighting device.

Note that the description of these objects does not preclude the existence of other objects. In one embodiment of the present invention, there is no need to achieve all these objects. Other objects are apparent from and can be derived from the description of the specification, the drawings, the claims, and the like.

### Means for Solving the Problems

(1) One embodiment of the present invention is a light-emitting device including a function of emitting light, a first electrode, a second electrode, and a unit. The light has a first spectrum ϕ1, and the first spectrum ϕ1 has a maximum peak at a wavelength λ.

The second electrode includes a region overlapping with the first electrode, the unit includes a region positioned between the first electrode and the second electrode, and the unit includes a first layer, a second layer, and a third layer.

The first layer includes a region positioned between the second layer and the third layer, and the first layer contains a light-emitting material.

The second layer includes a fourth layer and a fifth layer, and the fifth layer includes a region positioned between the fourth layer and the first layer.

The fourth layer contains a first organic compound CTM1, and the first organic compound CTM1 has a first refractive index n1 with respect to light having a wavelength λ1 nm.

The fifth layer is in contact with the fourth layer, and the fifth layer contains a second organic compound CTM2. The second organic compound CTM2 has a second refractive index n2 with respect to light having the wavelength λ, and the second refractive index n2 is higher than or equal to 1.4 and lower than or equal to 1.75.

(2) One embodiment of the present invention is a light-emitting device including a function of emitting light, a first electrode, a second electrode, and a unit. The light has a first spectrum ϕ1, and the first spectrum ϕ1 has a maximum peak at a wavelength λ1 nm.

The second electrode includes a region overlapping with the first electrode, the unit includes a region positioned between the first electrode and the second electrode, and the unit includes a first layer, a second layer, and a third layer.

The first layer includes a region positioned between the second layer and the third layer, and the first layer contains a light-emitting material.

The second layer includes a fourth layer and a fifth layer, and the fifth layer includes a region positioned between the fourth layer and the first layer,

The fourth layer contains a first organic compound CTM1, and the first organic compound CTM1 has a first refractive index n1 with respect to light having the wavelength λ1 nm.

The fifth layer is in contact with the fourth layer, the fifth layer contains a second organic compound CTM2, and the second organic compound CTM2 has a second refractive index n2 with respect to light having the wavelength λ1 nm. The second refractive index n2 is lower than the first refractive index n1.

(3) One embodiment of the present invention is the above-described light-emitting device in which the first refractive index n1 differs from the second refractive index n2 by 0.1 or more and 1.0 or less.

(4) One embodiment of the present invention is a light-emitting device including a first electrode, a second electrode, and a unit.

The second electrode includes a region overlapping with the first electrode, the unit includes a region positioned between the first electrode and the second electrode, and the unit includes a first layer, a second layer, and a third layer.

The first layer includes a region positioned between the second layer and the third layer, the first layer contains a light-emitting material, and the first layer emits photoluminescent light. The photoluminescent light has a second spectrum ϕ2, and the second spectrum ϕ2 has a maximum peak at a wavelength λ2 nm.

The second layer includes a region positioned between the first electrode and the first layer, the second layer includes a fourth layer and a fifth layer, and the fifth layer includes a region positioned between the fourth layer and the first layer.

The fourth layer contains a first organic compound CTM1, and the first organic compound CTM1 has a first refractive index n1 with respect to light having the wavelength λ2 nm.

The fifth layer is in contact with the fourth layer, and the fifth layer contains a second organic compound CTM2. The second organic compound CTM2 has a second refractive index n2 with respect to light having the wavelength λ2 nm, and the second refractive index n2 is higher than or equal to 1.4 and lower than or equal to 1.75.

(5) One embodiment of the present invention is a light-emitting device including a first electrode, a second electrode, and a unit.

The second electrode includes a region overlapping with the first electrode, the unit includes a region positioned between the first electrode and the second electrode, and the unit includes a first layer, a second layer, and a third layer.

The first layer includes a region positioned between the second layer and the third layer, the first layer contains a light-emitting material, and the first layer emits photoluminescent light. The photoluminescent light has a second spectrum ϕ2, and the second spectrum ϕ2 has a maximum peak at a wavelength λ2 nm.

The second layer includes a fourth layer and a fifth layer, and the fifth layer includes a region positioned between the fourth layer and the first layer.

The fourth layer contains a first organic compound CTM1, and the first organic compound CTM1 has a first refractive index n1 with respect to light having the wavelength λ2 nm.

The fifth layer is in contact with the fourth layer, the fifth layer contains a second organic compound CTM2, and the second organic compound CTM2 has a second refractive index n2 with respect to light having the wavelength λ2 nm. The second refractive index n2 is lower than the first refractive index n1.

(6) One embodiment of the present invention is the above-described light-emitting device in which the first refractive index n1 differs from the second refractive index n2 by 0.1 or more and 1.0 or less.

(7) One embodiment of the present invention is a light-emitting device including a first electrode, a second electrode, and a unit.

The second electrode includes a region overlapping with the first electrode, the unit includes a region positioned between the first electrode and the second electrode, and the unit includes a first layer, a second layer, and a third layer.

The first layer includes a region positioned between the second layer and the third layer, the first layer contains a light-emitting material, and the light-emitting material emits photoluminescent light. The photoluminescent light has a third spectrum ϕ3, and the third spectrum ϕ3 has a maximum peak at a wavelength λ3 nm.

The second layer includes a region positioned between the first electrode and the first layer, the second layer includes a fourth layer and a fifth layer, and the fifth layer includes a region positioned between the fourth layer and the first layer.

The fourth layer contains a first organic compound CTM1, and the first organic compound CTM1 has a first refractive index n1 with respect to light having the wavelength λ3 nm.

The fifth layer is in contact with the fourth layer, and the fifth layer contains a second organic compound CTM2. The second organic compound CTM2 has a second refractive index n2 with respect to light having the wavelength λ3 nm, and the second refractive index n2 is higher than or equal to 1.4 and lower than or equal to 1.75.

(8) One embodiment of the present invention is a light-emitting device including a first electrode, a second electrode, and a unit.

The second electrode includes a region overlapping with the first electrode, the unit includes a region positioned between the first electrode and the second electrode, and the unit includes a first layer, a second layer, and a third layer.

The first layer includes a region positioned between the second layer and the third layer, the first layer contains a light-emitting material, and the light-emitting material emits photoluminescent light. The photoluminescent light has a third spectrum ϕ3, and the third spectrum ϕ3 has a maximum peak at a wavelength λ3 nm.

The second layer includes a fourth layer and a fifth layer, and the fifth layer includes a region positioned between the fourth layer and the first layer.

The fourth layer contains a first organic compound CTM1, and the first organic compound CTM1 has a first refractive index n1 with respect to light having the wavelength λ3 nm.

The fifth layer is in contact with the fourth layer, the fifth layer contains a second organic compound CTM2, and the second organic compound CTM2 has a second refractive index n2 with respect to light having the wavelength λ3 nm. The second refractive index n2 is lower than the first refractive index n 1.

(9) One embodiment of the present invention is the above-described light-emitting device in which the first refractive index n1 differs from the second refractive index n2 by 0.1 or more and 1.0 or less.

Accordingly, the refractive index can be changed between the fourth layer and the fifth layer. Alternatively, light can be reflected with the use of the change in refractive index. Alternatively, light emitted from the first layer can be intensified with the use of the reflected light. Alternatively, the efficiency of extracting light from the light-emitting device can be increased. Alternatively, the emission efficiency of the light-emitting device can be increased.

Consequently, a novel light-emitting device that is highly convenient, useful, or reliable can be provided.

(10) One embodiment of the present invention is the above-described light-emitting 30 device in which the fourth layer has a distance d between the fourth layer and the first layer, and the distance is greater than or equal to 20 nm and less than or equal to 120 nm.

(11) One embodiment of the present invention is the above-described light-emitting device in which the fourth layer has a distance d between the fourth layer and the first layer, the first layer has a thickness t, and the distance d is in a range represented by the thickness t, the wavelength λ1 nm, the second refractive index n2, and the following Formula (1).

$$[ Formula 1 ]$$

$$0.5 \times 0.25 \times λ 1 \leq ( d + \frac{t}{2} ) \times n 2 \leq 1.5 \times 0.25 \times λ 1 \qquad ( 1 )$$

Accordingly, the refractive index can be changed between the fourth layer and the fifth layer. Alternatively, light can be reflected with the use of the change in refractive index. Alternatively, the phase of the reflected light can be made to be a phase with which the reflected light and light emitted from the first layer intensify each other. Alternatively, part of a microcavity structure can be formed inside the unit. Alternatively, the saturation of an emission color can be increased. Alternatively, the efficiency of extracting light from the light-emitting device can be increased. Alternatively, the emission efficiency of the light-emitting device can be increased. Consequently, a novel light-emitting device that is highly convenient, useful, or reliable can be provided.

(12) One embodiment of the present invention is the above-described light-emitting device in which the fifth layer is in contact with the first layer, and the fifth layer has a function of inhibiting transport of carriers from the first layer toward the fourth layer.

(13) One embodiment of the present invention is the above-described light-emitting device in which the second organic compound CTM2 has a hole-transport property.

The second organic compound CTM2 has a first lowest unoccupied molecular orbital level (abbreviation: LUMO level), the first layer contains a host material, the host material has a second LUMO level, and the second LUMO level is lower than the first LUMO level.

(14) One embodiment of the present invention is the above-described light-emitting device in which the second organic compound CTM2 is an amine compound.

(15) One embodiment of the present invention is the above-described light-emitting device in which the first organic compound CTM1 is an amine compound.

(16) One embodiment of the present invention is the above-described light-emitting device in which the second organic compound CTM2 is a monoamine compound.

The monoamine compound includes a group of aromatic groups and a nitrogen atom; and the group of aromatic groups includes a first aromatic group, a second aromatic group, and a third aromatic group.

The nitrogen atom is bonded to the first aromatic group, the second aromatic group, and the third aromatic group; the group of aromatic groups includes a substituent; and the substituent includes sp3 carbon. The sp3 carbon forms a bond with another atom by an sp3 hybrid orbital, and the sp3 carbon accounts for higher than or equal to 23% and lower than or equal to 55% of carbon included in the monoamine compound.

(17) One embodiment of the present invention is a light-emitting apparatus including the above light-emitting device and a transistor or a substrate.

(18) One embodiment of the present invention is a display apparatus including the above light-emitting device and a transistor or a substrate.

(19) One embodiment of the present invention is a lighting device including the above light-emitting apparatus and a housing.

(20) One embodiment of the present invention is an electronic device including the above display apparatus and a sensor, an operation button, a speaker, or a microphone.

Although a block diagram in which components are classified by their functions and shown as independent blocks is shown in the drawing attached to this specification, it is difficult to completely separate actual components according to their functions and one component can relate to a plurality of functions.

Note that the light-emitting apparatus in this specification includes an image display device using a light-emitting element. Moreover, the light-emitting apparatus may also include a module in which a connector such as an anisotropic conductive film or a TCP (Tape Carrier Package) is attached to a light-emitting element, a module in which a printed wiring board is provided on the tip of a TCP, or a module in which an IC (integrated circuit) is directly mounted on a light-emitting element by a COG (Chip On Glass) method. Furthermore, a lighting device or the like may include the light-emitting apparatus.

### Effect of the Invention

According to one embodiment of the present invention, a novel light-emitting device that is highly convenient, useful, or reliable can be provided. Alternatively, a novel light-emitting apparatus that is highly convenient, useful, or reliable can be provided. Alternatively, a novel display apparatus that is highly convenient, useful, or reliable can be provided. Alternatively, a novel electronic device that is highly convenient, useful, or reliable can be provided. Alternatively, a novel lighting device that is highly convenient, useful, or reliable can be provided. Alternatively, a novel light-emitting device, a novel light-emitting apparatus, a novel display apparatus, a novel electronic device, or a novel lighting device can be provided.

Note that the description of these effects does not preclude the existence of other effects. One embodiment of the present invention does not need to have all these effects. Other effects will be apparent from and can be derived from the description of the specification, the drawings, the claims, and the like.

### BRIEF DESCRIPTION OF THE DRAWINGS

FIG. 1A to FIG. 1D are diagrams illustrating a structure of a light-emitting device of an embodiment.

FIG. 2A and FIG. 2B are diagrams each illustrating a structure of a light-emitting device of an embodiment.

FIG. 3 is a diagram illustrating a structure of a functional panel of an embodiment.

FIG. 4A and FIG. 4B are conceptual diagrams of an active matrix light-emitting apparatus.

FIG. 5A and FIG. 5B are conceptual diagrams of active matrix light-emitting apparatuses.

FIG. 6 is a conceptual diagram of an active matrix light-emitting apparatus.

FIG. 7A and FIG. 7B are conceptual diagrams of a passive matrix light-emitting apparatus.

FIG. 8A and FIG. 8B are diagrams illustrating a lighting device.

FIG. 9A, FIG. 9B1, FIG. 9B2, and FIG. 9C are diagrams illustrating electronic devices.

FIG. 10A to FIG. 10C are diagrams illustrating electronic devices.

FIG. 11 is a diagram illustrating a lighting device.

FIG. 12 is a diagram illustrating a lighting device.

FIG. 13 is a diagram illustrating in-vehicle display apparatuses and lighting devices.

FIG. 14A to FIG. 14C are diagrams illustrating an electronic device.

FIG. 15A to FIG. 15C are diagrams illustrating structures of light-emitting devices of an example.

FIG. 16 is a graph showing current density—luminance characteristics of light-emitting devices of an example.

FIG. 17 is a graph showing luminance—current efficiency characteristics of light-emitting devices of an example.

FIG. 18 is a graph showing voltage—luminance characteristics of light-emitting devices of an example.

FIG. 19 is a graph showing voltage—current characteristics of light-emitting devices of an example.

FIG. 20 is a graph showing luminance—blue index characteristics of light-emitting devices of an example.

FIG. 21 is a graph showing emission spectra of light-emitting devices of an example.

FIG. 22 is a graph showing current density—luminance characteristics of a light-emitting device of an example.

FIG. 23 is a graph showing luminance—current efficiency characteristics of a light-emitting device of an example.

FIG. 24 is a graph showing voltage—luminance characteristics of a light-emitting device of an example.

FIG. 25 is a graph showing voltage—current characteristics of a light-emitting device of an example.

FIG. 26 is a graph showing luminance—external quantum efficiency characteristics of a light-emitting device of an example.

FIG. 27 is a graph showing an emission spectrum of a light-emitting device of an example.

### MODE FOR CARRYING OUT THE INVENTION

A light-emitting device includes a function of emitting light, a first electrode, a second electrode, and a unit; the light has the maximum peak at a wavelength λ; the second electrode includes a region overlapping with the first electrode; and the unit includes a region positioned between the first electrode and the second electrode. The unit includes a first layer, a second layer, and a third layer; the first layer includes a region positioned between the second layer and the third layer; and the first layer contains a light-emitting material. The second layer includes a fourth layer and a fifth layer, and the fifth layer includes a region positioned between the fourth layer and the first layer. The fourth layer contains a first organic compound; the first organic compound has a first refractive index with respect to light having the wavelength λ; the fifth layer is in contact with the fourth layer; the fifth layer contains a second organic compound; the second organic compound has a second refractive index with respect to light having the wavelength λ; and the second refractive index is lower than the first refractive index.

Accordingly, light emission efficiency can be increased. Alternatively, reliability as well as light emission efficiency can be increased. Consequently, a novel light-emitting device that is highly convenient, useful, or reliable can be provided.

Embodiments are described in detail with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments. Note that in structures of the invention described below, the same portions or portions having similar functions are denoted by the same reference numerals in different drawings, and a description thereof is not repeated.

### Embodiment 1

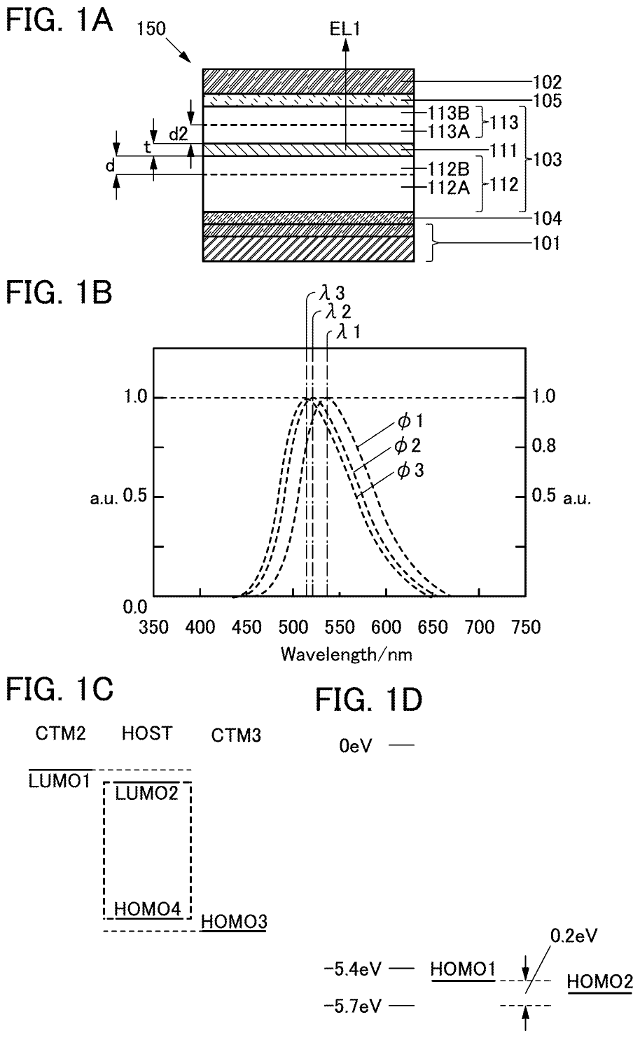

In this embodiment, a structure of a light-emitting device 150 of one embodiment of the present invention is described with reference to FIG. 1.

FIG. 1A is a diagram illustrating structures of light-emitting devices of one embodiment of the present invention, FIG. 1B is a graph showing spectra of light emitted from the light-emitting devices of one embodiment of the present invention, and FIG. 1C is a diagram illustrating part of the structure in FIG. 1A.

### <Structure Example 1 of Light-Emitting Device 150>

The light-emitting device 150 described in this embodiment includes a function of emitting light EL1, an electrode 101, an electrode 102, and a unit 103 (see FIG. 1A). Note that the light EL1 has a spectrum ϕ1, and the spectrum ϕ1 has a maximum peak at a wavelength λ1 nm (see FIG. 1B). The electrode 102 includes a region overlapping with the electrode 101.

### <<Structure Example 1 of Unit 103>>

The unit 103 includes a region positioned between the electrode 101 and the electrode 102, and the unit 103 includes a layer 111, a layer 112, and a layer 113.

### <<Structure Example 1 of Layer 111>>

The layer 111 includes a region positioned between the layer 112 and the layer 113, and the layer 111 contains a host material and a light-emitting material.

### <<Structure Example 1 of Layer 112>>

For example, a material having a carrier-transport property can be used for the layer 112. Specifically, a material having a hole-transport property can be used for the layer 112. A material having a wider band gap than the light-emitting material contained in the layer 111 is preferably used for the layer 112. Thus, energy transfer from excitons generated in the layer 111 to the layer 112 can be inhibited.

### [Example 1 of Material Having Hole-Transport Property]

A material having a hole mobility of 1×10⁻⁶ cm²/Vs or higher can be suitably used as the material having a hole-transport property.

As the material having a hole-transport property, an amine compound or an organic compound having a π-electron rich heteroaromatic ring skeleton can be used, for example. Specifically, a compound having an aromatic amine skeleton, a compound having a carbazole skeleton, a compound having a thiophene skeleton, a compound having a furan skeleton, or the like can be used. In particular, a compound having an aromatic amine skeleton and a compound having a carbazole skeleton are preferable because these have favorable reliability, have high hole-transport properties, and contribute to a reduction in driving voltage.

The following are examples that can be used as a compound having an aromatic amine skeleton: 4,4′-bis[N-(1-naphthyl)-N-phenylamino]biphenyl (abbreviation: NPB), N,N-bis(3-methylphenyl)-N,N-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]spiro-9,9′-bifluoren-2-amine (abbreviation: PCBASF).

As a compound having a carbazole skeleton, for example, 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), or the like can be used.

As a compound having a thiophene skeleton, for example, 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), or the like can be used.

As a compound having a furan skeleton, for example, 4,4′,4″-(benzene-1,3,5-triyl)tri(dibenzofuran) (abbreviation: DBF3P-II), 4-{3-[3-(9-phenyl-9H-fluoren-9-yl)phenyl]phenyl}dibenzofuran (abbreviation: mmDBFFLBi-II), or the like can be used.

### <<Structure Example 2 of Layer 112>>

The layer 112 includes a layer 112A and a layer 112B, and the layer 112B includes a region positioned between the layer 112A and the layer 111.

### <<Structure Example 1 of Layer 112A>>

The layer 112A contains a material CTM1. The material CTM1 has a refractive index n1 with respect to light having the wavelength λ1 nm.

### <<Structure Example 1 of Layer 112B>>

The layer 112B is in contact with the layer 112A, and the layer 112B contains a material CTM2. The material CTM2 has a refractive index n2 with respect to light having the wavelength λ1 nm, and the refractive index n2 is lower than the refractive index n1.

Accordingly, the refractive index can be changed between the layer 112A and the layer 112B. Alternatively, light can be reflected with the use of the change in refractive index. Alternatively, light emitted from the layer 111 can be intensified with the use of the reflected light. Alternatively, the efficiency of extracting light from the light-emitting device can be increased. Alternatively, the emission efficiency of the light-emitting device can be increased. Consequently, a novel light-emitting device that is highly convenient, useful, or reliable can be provided.

### <<Example 1 of Material CTM2>>

A material having a refractive index higher than or equal to 1.4 and lower than or equal to 1.75 can be favorably used as the material CTM2.

As the material CTM2, it is possible to use, for example, a material that has a hole-transport property and an ordinary refractive index higher than or equal to 1.50 and lower than or equal to 1.75 in a blue light emission range (455 nm to 465 nm) or an ordinary refractive index higher than or equal to 1.45 and lower than or equal to 1.70 with respect to 633-nm light, which is usually used for measurement of refractive indices.

In the case where the material has anisotropy, the refractive index with respect to an ordinary ray might differ from the refractive index with respect to an extraordinary ray. When a thin film to be measured is in such a state, anisotropy analysis can be performed to separately calculate the ordinary refractive index and the extraordinary refractive index. In this specification, when the measured material has both the ordinary refractive index and the extraordinary refractive index, the ordinary refractive index is used as an indicator.

### [Example 2 of Material Having Hole-Transport Property]

An example of the material having a hole-transport property is a monoamine compound including a first aromatic group, a second aromatic group, and a third aromatic group, in which the first aromatic group, the second aromatic group, and the third aromatic group are bonded to the same nitrogen atom.

In the monoamine compound, the proportion of carbon atoms forming a bond by the sp3 hybrid orbitals to the total number of carbon atoms in the molecule is preferably higher than or equal to 23% and lower than or equal to 55%. In addition, it is preferable that the integral value of signals at lower than 4 ppm exceed the integral value of signals at 4 ppm or higher in the results of ¹H-NMR measurement conducted on the monoamine compound.

The monoamine compound preferably has at least one fluorene skeleton. One or more of the first aromatic group, the second aromatic group, and the third aromatic group are preferably a fluorene skeleton.

Examples of the above-described material having a hole-transport property include organic compounds having structures represented by General Formulae (Gₕ₁1) to (Gₕ₁4) below.

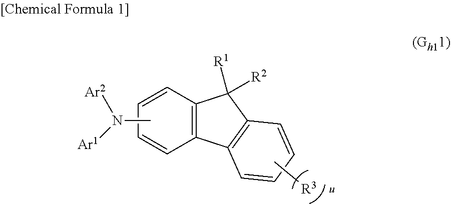

In General Formula (Gₕ₁1) above, each of Ar¹ and Ar² independently represents a benzene ring or a substituent in which two or three benzene rings are bonded to each other. Note that one or both of Ar¹ and Ar² have one or more hydrocarbon groups each having 1 to 12 carbon atoms forming a bond only by the sp3 hybrid orbitals. The total number of carbon atoms contained in all of the hydrocarbon groups bonded to Ar¹ and Ar² is 8 or more, and the total number of carbon atoms contained in all of the hydrocarbon groups bonded to either Ar¹ or Ar² is 6 or more. Note that in the case where a plurality of straight-chain alkyl groups each having one or two carbon atoms are bonded to Ar¹ or Ar² as the hydrocarbon groups, the straight-chain alkyl groups may be bonded to each other to form a ring.

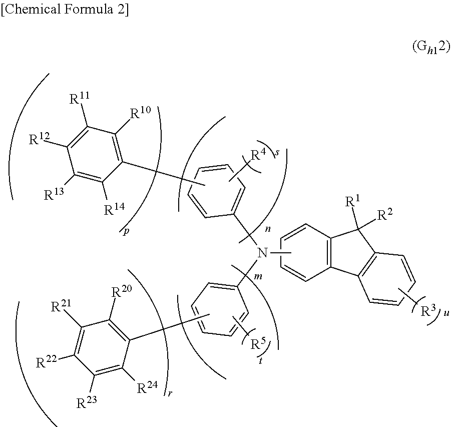

In General Formula (Gₕ₁2) above, each of m and r independently represents 1 or 2 and m+r is 2 or 3. Furthermore, t represents an integer of 0 to 4 and is preferably 0. Moreover, R⁵ represents hydrogen or a hydrocarbon group having 1 to 3 carbon atoms. When m is 2, the kind and number of sub stituents and the position of bonds included in one phenylene group may be the same as or different from those of the other phenylene group; and when r is 2, the kind and number of substituents and the position of bonds included in one phenyl group may be the same as or different from those of the other phenyl group. In the case where t is an integer of 2 to 4, R⁵s may be the same as or different from each other, and adjacent groups of R⁵s may be bonded to each other to form a ring.

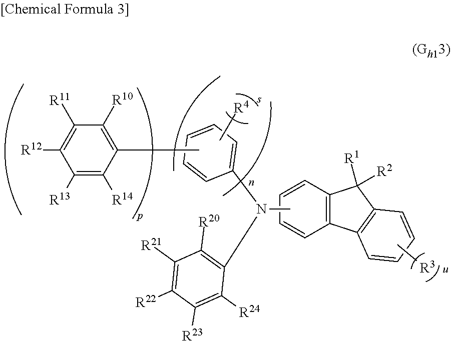

In General Formulae (Gₕ₁2) and (Gₕ₁3) above, each of n and p independently represents 1 or 2 and n+p is 2 or 3. In addition, s represents an integer of 0 to 4 and is preferably 0. Moreover, R⁴ represents hydrogen or a hydrocarbon group having 1 to 3 carbon atoms. When n is 2, the kind and number of substituents and the position of bonds in one phenylene group may be the same as or different from those of the other phenylene group; and when p is 2, the kind and number of substituents and the position of bonds in one phenyl group may be the same as or different from those of the other phenyl group. In the case where s is an integer of 2 to 4, R⁴s may be the same as or different from each other.

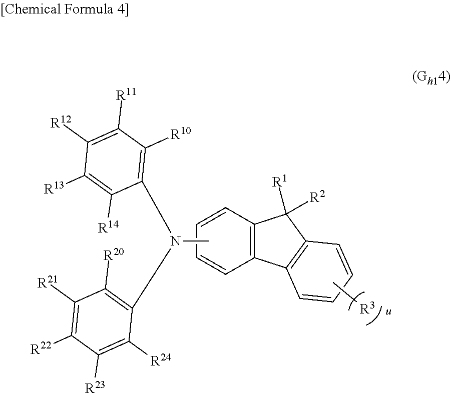

In General Formulae (Gₕ₁2) to (Gₕ₁4) above, each of R¹⁰ to R¹⁴ and R²⁰ to R²⁴ independently represents hydrogen or a hydrocarbon group having 1 to 12 carbon atoms forming a bond only by the sp3 hybrid orbitals. Note that at least three of R¹⁰ to R¹⁴ and at least three of R²⁰ to R²⁴ are preferably hydrogen. As the hydrocarbon group having 1 to 12 carbon atoms forming a bond only by the sp3 hybrid orbitals, a tert-butyl group and a cyclohexyl group are preferable. The total number of carbon atoms contained in R¹⁰ to R¹⁴ and R²⁰ to R²⁴ is 8 or more, and the total number of carbon atoms contained in either R¹⁰ to R¹⁴ or R²⁰ to R²⁴ is 6 or more. Adjacent groups of R⁴, R¹⁰ to R¹⁴, and R²⁰ to R²⁴ may be bonded to each other to form a ring.

In General Formulae (Gₕ₁1) to (Gₕ₁4) above, u represents an integer of 0 to 4 and is preferably 0. In the case where u is an integer of 2 to 4, les may be the same as or different from each other. In addition, each of R¹, R², and R³ independently represents an alkyl group having 1 to 4 carbon atoms, and R¹ and R² may be bonded to each other to form a ring.

An arylamine compound that has at least one aromatic group having first to third benzene rings and at least three alkyl groups is preferable as one of the materials having a hole-transport property. Note that the first to third benzene rings are bonded in this order, and the first benzene ring is directly bonded to nitrogen of amine.

The first benzene ring may further include a substituted or unsubstituted phenyl group and preferably includes an unsubstituted phenyl group. Furthermore, the second benzene ring or the third benzene ring may include a phenyl group substituted by an alkyl group.

Note that hydrogen is not directly bonded to carbon atoms at 1- and 3-positions in two or more of, preferably all of the first to third benzene rings, and the carbon atoms are bonded to any of the first to third benzene rings, the phenyl group substituted by the alkyl group, the at least three alkyl groups, and the nitrogen of the amine.

It is preferable that the arylamine compound further include a second aromatic group. The second aromatic group is preferably a group having an unsubstituted monocyclic ring or a substituted or unsubstituted bicyclic or tricyclic condensed ring, further preferably a group having a substituted or unsubstituted bicyclic or tricyclic condensed ring where the number of carbon atoms forming the ring is 6 to 13, still further preferably a group including a fluorene ring. Note that a dimethylfluorenyl group is preferable as the second aromatic group.

It is preferable that the arylamine compound further include a third aromatic group. The third aromatic group is a group having 1 to 3 substituted or unsubstituted benzene rings.

It is preferable that the at least three alkyl groups and the alkyl group substituted for the phenyl group be each a chain alkyl group having 2 to 5 carbon atoms. In particular, as the alkyl group, a chain alkyl group having a branch formed of 3 to 5 carbon atoms is preferable, and a t-butyl group is further preferable.

Examples of the above-described material having a hole-transport property include organic compounds having structures represented by (G^{h2}1) to (Gₕ₂3) shown below.

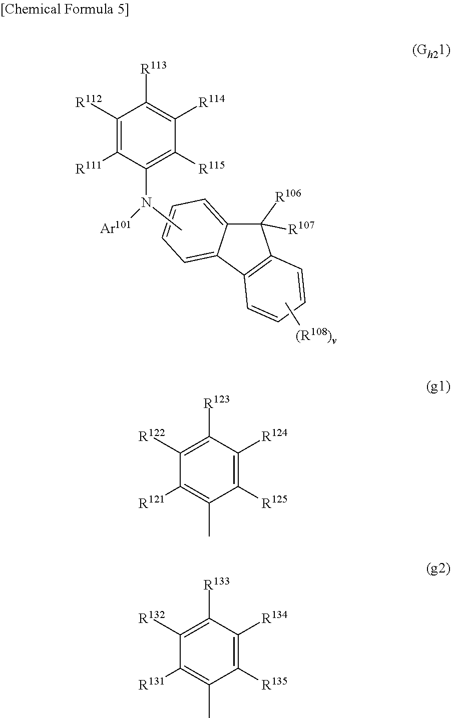

Note that in General Formula (G^{h2}1) above, Ar¹⁰¹ represents a substituted or unsubstituted benzene ring or a substituent in which two or three substituted or unsubstituted benzene rings are bonded to each other.

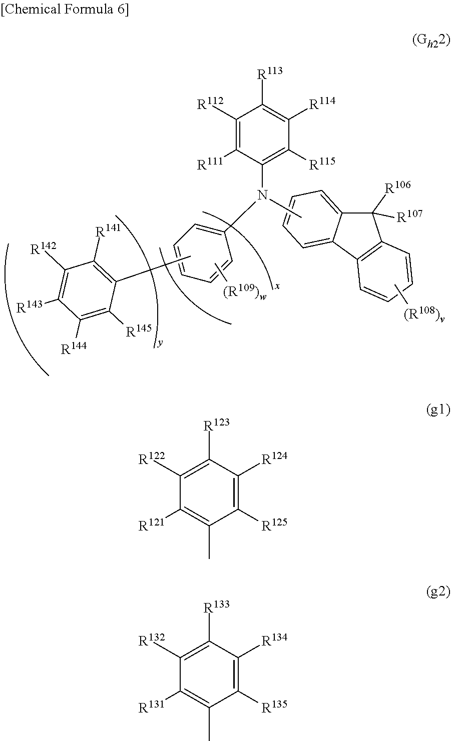

Note that in General Formula (Gₕ₂2) above, each of x and y independently represents 1 or 2 and x+y is 2 or 3. Furthermore, R¹⁰⁹ represents an alkyl group having 1 to 4 carbon atoms, and w represents an integer of 0 to 4. Each of R¹⁴¹ to R¹⁴⁵ independently represents any one of hydrogen, an alkyl group having 1 to 6 carbon atoms, and a cycloalkyl group having 5 to 12 carbon atoms. When w is 2 or more, R¹⁰⁹ may be the same as or different from each other. When x is 2, the kind and number of substituents and the position of bonds included in one phenylene group may be the same as or different from those of the other phenylene group. When y is 2, the kind and number of substituents included in one phenyl group including R¹⁴¹ to R¹⁴⁵ may be the same as or different from those of the other phenyl group including R¹⁴¹ to R¹⁴⁵.

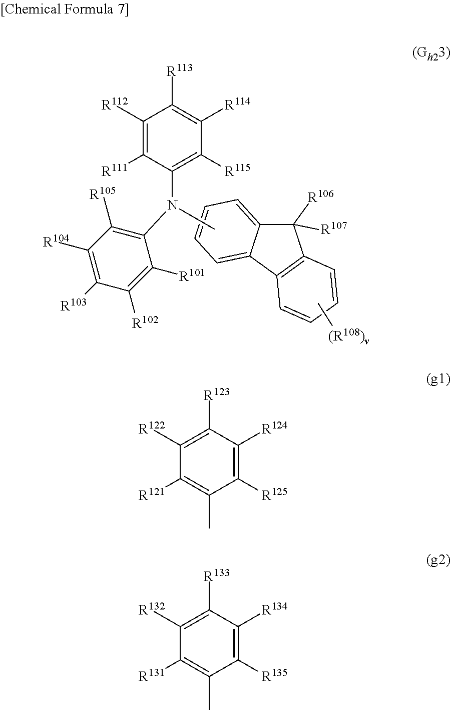

In General Formula (Gₕ₂3) above, each of R¹⁰¹ to R¹⁰⁵ independently represents any one of hydrogen, an alkyl group having 1 to 6 carbon atoms, a cycloalkyl group having 6 to 12 carbon atoms, and a substituted or unsubstituted phenyl group.

In General Formulae (Gₕ₂1) to (Gₕ₂3) above, each of R¹⁰⁶, R¹⁰⁷, and R¹⁰⁸ independently represents an alkyl group having 1 to 4 carbon atoms, and v represents an integer of 0 to 4. Note that when v is 2 or more, R¹⁰⁸s may be the same as or different from each other. One of R¹¹¹ to R¹¹⁵ represents a substituent represented by General Formula (g1) above, and the others each independently represent any one of hydrogen, an alkyl group having 1 to 6 carbon atoms, and a substituted or unsubstituted phenyl group. In General Formula (g1) above, one of R¹²¹ to R¹²⁵ represents a substituent represented by General Formula (g2) above, and the others each independently represent any one of hydrogen, an alkyl group having 1 to 6 carbon atoms, and a phenyl group substituted by an alkyl group having 1 to 6 carbon atoms. In General Formula (g2) above, each of R¹³¹ to R¹³⁵ independently represents any one of hydrogen, an alkyl group having 1 to 6 carbon atoms, and a phenyl group substituted by an alkyl group having 1 to 6 carbon atoms. Note that at least three of R¹¹¹ to R¹¹⁵, R¹²¹ to R¹²⁵, and R¹³¹ to R¹³⁵ are each an alkyl group having 1 to 6 carbon atoms; the number of substituted or unsubstituted phenyl groups in R¹¹¹ to R¹¹⁵ is one or less; and the number of phenyl groups substituted by an alkyl group having 1 to 6 carbon atoms in R¹²¹ to R¹²⁵ and R¹³¹ to R¹³⁵ is one or less. In at least two combinations of the three combinations R¹¹² and R¹¹⁴, R¹²² and R¹²⁴, and R¹³² and R¹¹⁴, at least one R represents any of the substituents other than hydrogen.

Specifically, any of the following can be used as the material CTM2, for example: N,N-bis(4-cyclohexylphenyl)-N-(9,9-dimethyl-9H-fluoren-2yl)amine (abbreviation: dchPAF), N-(4-cyclohexylphenyl)-N-(3″,5″-ditertiarybutyl-1,1″-biphenyl-4-yl)-N-(9,9-dimethyl-9H-fluoren-2yl)amine (abbreviation: mmtBuBichPAF), N-(3,3″,5,5″-tetra-t-butyl-1,1′:3′,1″-terphenyl-5′-yl)-N-(4-cyclohexylphenyl)-9,9-dimethyl-9H-fluoren-2-amine (abbreviation: mmtBumTPchPAF), N-[(3,3′,5′-t-butyl)-1,1′-biphenyl-5-yl]-N-(4-cyclohexylphenyl)-9,9-dimethyl-9H-fluoren-2-amine (abbreviation: mmtBumBichPAF), N-(1,1′-biphenyl-2-yl)-N-[(3,3′,5′-tri-t-butyl)-1,1′-biphenyl-5-yl]-9,9-dimethyl-9H-fluoren-2-amine (abbreviation: mmtBumBioFBi), N-(4-tert-butylphenyl)-N-(3,3″,5,5″-tetra-t-butyl-1,1′:3′,1″-terphenyl-5′-yl)-9,9,-dimethyl-9H-fluoren-2-amine (abbreviation: mmtBumTPtBuPAF), N-(1,1′-biphenyl-2-yl)-N-(3,3″,5′,5″-tetra-t-butyl-1,1′:3′,1″-terphenyl-5-yl)-9,9-dimethyl-9H-fluoren-2-amine (abbreviation: mmtBumTPoFBi-02), N-(4-cyclohexylphenyl)-N-(3,3″,5′,5″-tetra-t-butyl-1,1′:3′,1″-terphenyl-5-yl)-9,9-dimethyl-9H-fluoren-2-amine (abbreviation: mmtBumTPchPAF-02), N-(1,1′-biphenyl-2-yl)-N-(3″,5′,5″-tri-t-butyl-1,1′:3′,1″-terphenyl-5-yl)-9,9-dimethyl-9H-fluoren-2-amine (abbreviation: mmtBumTPoFBi-03), and N-(4-cyclohexylphenyl)-N-(3″,5′,5″-tri-t-butyl-1,1′:3′,1″-terphenyl-5-yl)-9,9-dimethyl-9H-fluoren-2-amine (abbreviation: mmtBumTPchPAF-03).

### <<Example of Material CTM1>>

A material whose refractive index differs from the refractive index n2 of the material CTM2 by 0.1 or more and 1.0 or less can be favorably used as the material CTM1. Preferably, a material whose refractive index differs from the refractive index n2 of the material CTM2 by 0.15 or more and 1.0 or less can be used as the material CTM1. Further preferably, a material whose refractive index differs from the refractive index n2 of the material CTM2 by 0.2 or more and 1.0 or less can be used as the material CTM1. Specifically, a material selected appropriately from the above-described materials having a hole-transport property can be used as the material CTM1.

### <Structure Example 2 of Light-Emitting Device 150>

The light-emitting device 150 described in this embodiment is different from Structure Example 1 of the light-emitting device 150 in that the layer 111 emits photoluminescent light and the photoluminescent light has a second spectrum ϕ2. Different portions will be described in detail here, and the above description is referred to for portions that can use similar structures.

### <<Structure Example 2 of Layer 111>>

The layer 111 emits photoluminescent light, and the photoluminescent light has the second spectrum ϕ2. Note that the second spectrum ϕ2 has a maximum peak at a wavelength λ2 nm.

### <<Structure Example 2 of Layer 112A>>

The layer 112A contains the material CTM1. The material CTM1 has the refractive index n1 with respect to light having the wavelength λ2 nm.

### <<Structure Example 2 of Layer 112B>>

The layer 112B is in contact with the layer 112A, and the layer 112B contains the material CTM2. The material CTM2 has the refractive index n2 with respect to light having the wavelength λ2 nm, and the refractive index n2 is lower than the refractive index n1.

### <Structure Example 3 of Light-Emitting Device 150>

The light-emitting device 150 described in this embodiment is different from Structure Example 1 of the light-emitting device 150 in that the layer 111 contains a light-emitting material, the light-emitting material emits photoluminescent light, and the photoluminescent light has a third spectrum ϕ3. Different portions will be described in detail here, and the above description is referred to for portions that can use similar structures.

### <<Structure Example 3 of Layer 111>>

The layer 111 contains a light-emitting material, and the light-emitting material emits photoluminescent light. The photoluminescent light has the third spectrum ϕ3. Note that the third spectrum ϕ3 has a maximum peak at a wavelength λ3 nm. Photoluminescence from the light-emitting material can be observed, for example, in a state where the light-emitting material is dissolved in a solvent. Photoluminescence from the light-emitting material can be observed, for example, in a state where the light-emitting material is dissolved in a polar solvent, a non-polar solvent, water, or the like. Specifically, toluene, dichloromethane, acetonitrile, or the like can be used as the solvent. In particular, toluene can be suitably used.

### <<Structure Example 3 of Layer 112A>>

The layer 112A contains the material CTM1. The material CTM1 has the refractive index n1 with respect to light having the wavelength λ3 nm.

### <<Structure Example 3 of Layer 112B>>

The layer 112B is in contact with the layer 112A, and the layer 112B contains the material CTM2. The material CTM2 has the refractive index n2 with respect to light having the wavelength λ3 nm, and the refractive index n2 is lower than the refractive index n1.

### <<Structure Example 4 of Layer 112A>>

The layer 112A has a distance d between the layer 112A and the layer 111. For example, the distance d is greater than or equal to 20 nm and less than or equal to 120 nm.

### <<Structure Example 2 of Unit 103>>

In the light-emitting device 150 described in this embodiment, the structure of the unit 103 has a relation represented by the following formula. Note that in the formula, d is the distance between the layer 112A and the layer 111, t is the thickness of the layer 111, λ is the wavelength of the maximum peak of the emission spectrum, and n2 is the refractive index of the material CTM2 with respect to light having the wavelength λ nm (see FIG. 1A).

$$[ Formula 2 ]$$

$$0.5 \times 0.25 \times λ \leq ( d + \frac{t}{2} ) \times n 2 \leq 1.5 \times 0.25 \times λ \qquad ( 1 )$$

Note that in the spectrum of light emitted from the light-emitting device 150, the wavelength λ1 nm, at which the maximum peak is observed, can be used as the wavelength λ nm. Alternatively, in the spectrum of photoluminescent light emitted from the layer 111, the wavelength λ2 nm, at which the maximum peak is observed, can be used as the wavelength λ nm. Alternatively, in the spectrum of photoluminescent light emitted from the light-emitting material contained in the layer 111, the wavelength λ3 nm, at which the maximum peak is observed, can be used as the wavelength λ nm.

Accordingly, the refractive index can be changed between the layer 112A and the layer 112B. Alternatively, light can be reflected with the use of the change in refractive index. Alternatively, the phase of the reflected light can be made to be a phase with which the reflected light and light emitted from the layer 111 intensify each other. Alternatively, part of a microcavity structure can be formed inside the unit 103. Alternatively, the saturation of an emission color can be increased. Alternatively, the efficiency of extracting light from the light-emitting device can be increased. Alternatively, the emission efficiency of the light-emitting device can be increased. Consequently, a novel light-emitting device that is highly convenient, useful, or reliable can be provided.

### <<Structure Example 4 of Layer 112>>

In one embodiment of the present invention, the layer 112B is in contact with the layer 111, and the layer 112B has a function of inhibiting transfer of carriers from the layer 111 toward the layer 112A. For example, the layer 112B has a function of inhibiting transfer of electrons.

### <<Example 2 of Material CTM2>>

The material CTM2 has a hole-transport property, and the material CTM2 has a LUMO level LUMO1 (see FIG. 1C).

### <<Structure Example 4 of Layer 111>>

The layer 111 contains a host material. The host material (HOST) has a LUMO level LUMO2, and the LUMO level LUMO2 is lower than the LUMO level LUMO1.

Accordingly, transfer of electrons from the layer 111 toward the layer 112A can be inhibited. Alternatively, the probability of recombination of electrons and holes in the layer 111 can be increased. Alternatively, the light emission efficiency can be increased. Alternatively, the reliability can be increased. Consequently, a novel light-emitting device that is highly convenient, useful, or reliable can be provided.

### <<Structure Example 1 of Layer 113>>

For example, a material having an electron-transport property, a material having an anthracene skeleton, and a mixed material can be used for the layer 113. The layer 113 can be referred to as an electron-transport layer. A material having a wider band gap than the light-emitting material contained in the layer 111 is preferably used for the layer 113. Thus, energy transfer from excitons generated in the layer 111 to the layer 113 can be inhibited.

### [Material Having Electron-Transport Property]

For example, a metal complex or an organic compound having a π-electron deficient heteroaromatic ring skeleton can be used as the material having an electron-transport property.

A material having an electron mobility higher than or equal to 1×10⁻⁷ cm²/Vs and lower than or equal to 5×10⁻⁵ cm²/Vs in a condition where the square root of the electric field strength [V/cm] is 600 can be favorably used as the material having an electron-transport property. Thus, the electron-transport property in the electron-transport layer can be controlled. Alternatively, the amount of electrons injected into the light-emitting layer can be controlled. Alternatively, the light-emitting layer can be prevented from having excess electrons.

As the metal complex, bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), bis[2-(2-benzothiazolyl)phenolato]zinc(II) (abbreviation: ZnBTZ), or the like can be used, for example.

As the organic compound having a π-electron deficient heteroaromatic ring skeleton, a heterocyclic compound having a polyazole skeleton, a heterocyclic compound having a diazine skeleton, a heterocyclic compound having a pyridine skeleton, a heterocyclic compound having a triazine skeleton, or the like can be used, for example. In particular, the heterocyclic compound having a diazine skeleton or the heterocyclic compound having a pyridine skeleton has favorable reliability and thus is preferable. Furthermore, the heterocyclic compound having a diazine (pyrimidine or pyrazine) skeleton has a high electron-transport property and contributes to a reduction in driving voltage.

As the heterocyclic compound having a polyazole skeleton, 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-b enzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or the like can be used, for example.

As the heterocyclic compound having a diazine skeleton, 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,8-bis[3-(dibenzothiophen-4-yl)phenyl]benzo[h]quinazoline (abbreviation: 4,8mDBtP2Bqn), or the like can be used, for example.

As the heterocyclic compound having a pyridine skeleton, 3,5-bis[3-(9H-carbazol-9-yl)phenyl]pyridine (abbreviation: 35DCzPPy), 1,3,5-tri[3-(3-pyridyl)phenyl]b enzene (abbreviation: TmPyPB), or the like can be used, for example.

As the heterocyclic compound having a triazine skeleton, 2-[3′-(9,9-dimethyl-9H-fluoren-2-yl)-1,1′-biphenyl-3-yl]-4,6-diphenyl-1,3,5-triazine (abbreviation: mFBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3 [3-(benzo[b]naphtho[1,2-d]furan-6-yl)phenyl]phenyl}-4,6-diphenyl-1,3,5-triazine (abbreviation: mBnfBPTzn-02), or the like can be used, for example.

### [Material Having Anthracene Skeleton]

An organic compound having an anthracene skeleton can be used for the layer 113. In particular, an organic compound having both an anthracene skeleton and a heterocyclic skeleton can be suitably used.

For example, an organic compound having both an anthracene skeleton and a nitrogen-containing five-membered ring skeleton can be used. Alternatively, an organic compound having both an anthracene skeleton and a nitrogen-containing five-membered ring skeleton where two heteroatoms are included in a ring can be used. Specifically, a pyrazole ring, an imidazole ring, an oxazole ring, a thiazole ring, or the like can be favorably used as the heterocyclic skeleton.

For example, an organic compound having both an anthracene skeleton and a nitrogen-containing six-membered ring skeleton can be used. Alternatively, an organic compound having both an anthracene skeleton and a nitrogen-containing six-membered ring skeleton where two heteroatoms are included in a ring can be used. Specifically, a pyrazine ring, a pyrimidine ring, a pyridazine ring, or the like can be favorably used as the heterocyclic skeleton.

### [Structure Example of Mixed Material]

A material in which a plurality of kinds of substances are mixed can be used for the layer 113. Specifically, a mixed material that contains a substance having an electron-transport property and any of an alkali metal, an alkali metal compound, and an alkali metal complex can be used for the layer 113. Note that it is further preferable that the highest occupied molecular orbital level (abbreviation: HOMO level) of the material having an electron-transport property be −6.0 eV or higher.

The mixed material can be suitably used for the layer 113 in combination with a structure using a composite material for a layer 104. For example, a composite material of a substance having an acceptor property and a material having a hole-transport property can be used for the layer 104. Specifically, a composite material of a substance having an acceptor property and a substance having a relatively deep HOMO level HOMO1, which is greater than or equal to −5.7 eV and lower than or equal to −5.4 eV, can be used for the layer 104 (see FIG. 1D). In particular, the mixed material can be suitably used for the layer 113 in combination with the structure using the composite material for the layer 104. As a result, the reliability of the light-emitting device can be increased.

Furthermore, a structure using a material having a hole-transport property for the layer 112 can be suitably combined with the structure using the mixed material for the layer 113 and the composite material for the layer 104. For example, a substance having the HOMO level HOMO2, which is within the range of ˜0.2 eV to 0 eV from the relatively deep HOMO level HOMO1, can be used for the layer 112 (see FIG. 1D). As a result, the reliability of the light-emitting device can be increased.

The concentration of the alkali metal, the alkali metal compound, or the alkali metal complex preferably differs in the thickness direction of the layer 113 (including the case where the concentration is 0).

For example, a metal complex having an 8-hydroxyquinolinato structure can be used. A methyl-substituted product of the metal complex having an 8-hydroxyquinolinato structure (e.g., a 2-methyl-substituted product or a 5-methyl-substituted product) or the like can also be used.

As the metal complex having an 8-hydroxyquinolinato structure, 8-hydroxyquinolinato-lithium (abbreviation: Liq), 8-hydroxyquinolinato-sodium (abbreviation: Naq), or the like can be used. In particular, a complex of a monovalent metal ion, especially a complex of lithium is preferable, and Liq is further preferable.

### <<Structure Example 2 of Layer 113>>

The layer 113 includes a layer 113A and a layer 113B, and the layer 113A includes a region positioned between the layer 113B and the layer 111.

### <<Structure Example 1 of Layer 113B>>

The layer 113B contains a material CTM12. The material CTM12 has a refractive index n12 with respect to light having the wavelength λ1 nm.

### <<Structure Example of Layer 113A>>

The layer 113A is in contact with the layer 113B, and the layer 113A contains a material CTM11. The material CTM11 has a refractive index n11 with respect to light having the wavelength λ1 nm, and the refractive index n11 is lower than the refractive index n12.

### <<Example 1 of Material CTM11>>

A material having a refractive index higher than or equal to 1.4 and lower than or equal to 1.75 can be favorably used as the material CTM11.

As the material CTM11, it is possible to use, for example, a material that has an electron-transport property and an ordinary refractive index higher than or equal to 1.50 and lower than or equal to 1.75 in a blue light emission range (455 nm to 465 nm) or an ordinary refractive index higher than or equal to 1.45 and lower than or equal to 1.70 with respect to 633-nm light, which is usually used for measurement of refractive indices.

In the case where the material has anisotropy, the refractive index with respect to an ordinary ray might differ from the refractive index with respect to an extraordinary ray. When a thin film to be measured is in such a state, anisotropy analysis can be performed to separately calculate the ordinary refractive index and the extraordinary refractive index. In this specification, when the measured material has both the ordinary refractive index and the extraordinary refractive index, the ordinary refractive index is used as an indicator.

### [Material Having Electron-Transport Property]

An example of the material having an electron-transport property is an organic compound that includes at least one six-membered heteroaromatic ring having 1 to 3 nitrogen atoms, a plurality of aromatic hydrocarbon rings each of which has 6 to 14 carbon atoms forming a ring and at least two of which are benzene rings, and a plurality of hydrocarbon groups forming a bond by the sp3 hybrid orbitals.

In such an organic compound, the proportion of carbon atoms forming a bond by the sp3 hybrid orbitals in total carbon atoms in the molecule of the organic compound is preferably higher than or equal to 10% and lower than or equal to 60%, further preferably higher than or equal to 10% and lower than or equal to 50%. Alternatively, when such an organic compound is subjected to ¹H-NMIR measurement, the integral value of signals at lower than 4 ppm is preferably ½ or more of the integral value of signals at 4 ppm or higher.

It is preferable that all the hydrocarbon groups forming a bond by the sp3 hybrid orbitals in the above organic compound be bonded to the aromatic hydrocarbon rings each having 6 to 14 carbon atoms forming a ring, and the LUMO of the organic compound not be distributed in the aromatic hydrocarbon rings.

The organic compound having an electron-transport property is preferably an organic compound represented by General Formula (Gₑ₁1) or (Gₑ₁2) shown below.

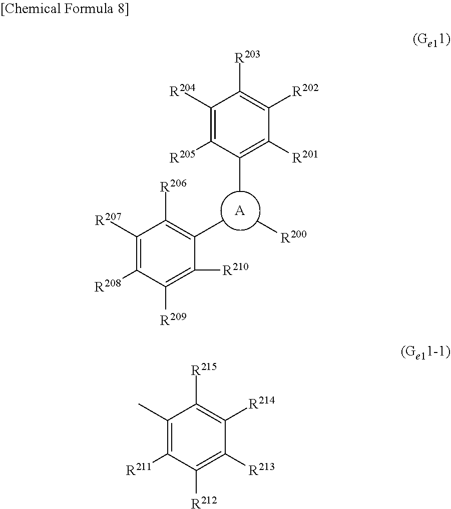

In the formula, A represents a six-membered heteroaromatic ring having 1 to 3 nitrogen atoms, and is preferably any of a pyridine ring, a pyrimidine ring, a pyrazine ring, a pyridazine ring, and a triazine ring.

In addition, R²⁰⁰ represents any of hydrogen, an alkyl group having 1 to 6 carbon atoms, an alicyclic group having 3 to 10 carbon atoms, and a substituent represented by Formula (Gₑ₁1-1).

At least one of R²⁰¹ to R²¹⁵ represents a phenyl group having a substituent and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. Note that R²⁰¹, R²⁰³, R²⁰⁵, R²⁰⁶, R²⁰⁸, R²¹⁰, R²¹¹, R²¹³, and R²¹⁵ are preferably hydrogen. The phenyl group having a substituent has one or two substituents, which each independently represent any of an alkyl group having 1 to 6 carbon atoms, an alicyclic group having 3 to 10 carbon atoms, and a substituted or unsubstituted aromatic hydrocarbon group having 6 to 14 carbon atoms in a ring.

The organic compound represented by General Formula (Gₑ₁1) shown above has a plurality of hydrocarbon groups selected from an alkyl group having 1 to 6 carbon atoms and an alicyclic group having 3 to 10 carbon atoms, and total carbon atoms forming a bond by the sp3 hybrid orbitals account for higher than or equal to 10% and lower than or equal to 60% of total carbon atoms in molecules of the organic compound.

The organic compound having an electron-transport property is preferably an organic compound represented by General Formula (Gₑ₁2) shown below.

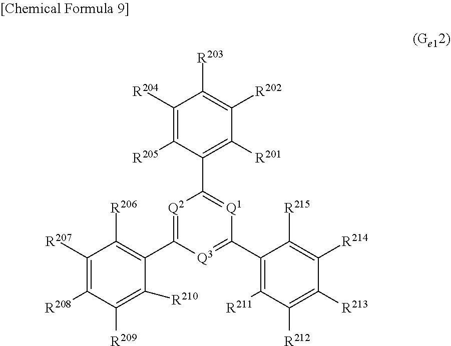

In the formula, two or three of Q¹ to Q³ represent N; in the case where two of Q¹ to Q³ are N, the other represents CH.

At least any one of R²⁰¹ to R²¹⁵ represents a phenyl group having a substituent and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in a ring, and a substituted or unsubstituted pyridyl group. Note that R²⁰¹, R²⁰³, R²⁰⁵, R²⁰⁶, R²⁰⁸, R²¹⁰, R²¹¹, R²¹³, and R²¹⁵ are preferably hydrogen.

The phenyl group having a substituent has one or two substituents, which each independently represent any of an alkyl group having 1 to 6 carbon atoms, an alicyclic group having 3 to carbon atoms, and a substituted or unsubstituted aromatic hydrocarbon group having 6 to 14 carbon atoms in a ring.

The organic compound represented by General Formula (Gₑ₁2) above includes a plurality of hydrocarbon groups selected from an alkyl group having 1 to 6 carbon atoms and an alicyclic group having 3 to 10 carbon atoms, and carbon atoms forming a bond by the sp3 hybrid orbitals preferably account for higher than or equal to 10% and lower than or equal to 60% of total carbon atoms in a molecule of the organic compound.

In the organic compound represented by General Formula (Gₑ₁1) or (Gₑ₁2) shown above, the phenyl group having a substituent is preferably a group represented by Formula (Gₑ₁1-2) shown below.

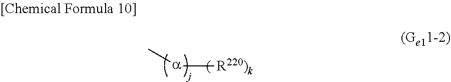

In the formula, a represents a substituted or unsubstituted phenylene group and is preferably a meta-substituted phenylene group. In the case where the meta-substituted phenylene group has a substituent, the substituent is also preferably meta-substituted. The substituent is preferably an alkyl group having 1 to 6 carbon atoms or an alicyclic group having 3 to 10 carbon atoms, further preferably an alkyl group having 1 to 6 carbon atoms, and still further preferably a t-butyl group.

R²²⁰ represents an alkyl group having 1 to 6 carbon atoms, an alicyclic group having 3 to 10 carbon atoms, or a substituted or unsubstituted aromatic hydrocarbon group having 6 to 14 carbon atoms in a ring.

In addition, j and k each represent 1 or 2. In the case where j is 2, a plurality of a may be the same or different from each other. In the case where k is 2, a plurality of R²²⁰ may be the same or different from each other. Note that R²²⁰ is preferably a phenyl group and is a phenyl group that has an alkyl group having 1 to 6 carbon atoms or an alicyclic group having 3 to 10 carbon atoms at one or both of the two meta-positons. The substituent at one or both of the two meta-positons of the phenyl group is preferably an alkyl group having 1 to 6 carbon atoms, further preferably a t-butyl group.

Specifically, any of the following can be used as the material CTM11, for example: 2-{(3′,5′-di-tert-butyl)-1,1′-biphenyl-3-yl}-4,6-bis(3,5-di-tert-butylphenyl)-1,3,5-triazine (abbreviation: mmtBumBP-dmmtBuPTzn), 2-{(3′,5′-di-tert-butyl)-1,1′-biphenyl-3-yl}-4,6-diphenyl-1,3,5-triazine (abbreviation: mmtBumBPTzn), 2-(3,3″,5,5″-tetra-tert-butyl-1,1′:3′,1″-phenyl-5′-yl)-4,6-diphenyl-1,3,5-triazine (abbreviation: mmtBumTPTzn), 2-{(3′,5′-di-tert-butyl)-1,1′-biphenyl-3-yl}-4,6-bis(3,5-di-tert-butylphenyl)-1,3-pyrimidine (abbreviation: mmtBumBP-dmmtBuPPm), and 2-(3,3″,5′,5″-tetra-tert-butyl-1,1′:3′,1″-terphenyl-5-yl)-4,6-diphenyl-1,3,5-triazine (abbreviation: mmtBumTPTzn-02).

### <<Example of Material CTM12>>

A material whose refractive index differs from the refractive index n11 of the material CTM11 by 0.1 or more and 1.0 or less can be favorably used as the material CTM12. Preferably, a material whose refractive index differs from the refractive index n11 of the material CTM11 by 0.15 or more and 1.0 or less can be used as the material CTM12. Further preferably, a material whose refractive index differs from the refractive index n11 of the material CTM11 by 0.2 or more and 1.0 or less can be used as the material CTM12. Specifically, a material selected appropriately from the above-described materials having an electron-transport property can be used as the material CTM12.

### <<Structure Example 2 of Layer 113B>>

The layer 113B has a distance d2 between the layer 113B and the layer 111. For example, the distance d2 is greater than or equal to 20 nm and less than or equal to 120 nm.

### <<Structure Example 3 of Unit 103>>

In the light-emitting device 150 described in this embodiment, the structure of the unit 103 has a relation represented by the following formula. Note that in the formula, d2 is the distance between the layer 113B and the layer 111, t is the thickness of the layer 111, λ is the wavelength of the maximum peak of the emission spectrum, and n11 is the refractive index of the material CTM11 with respect to light having the wavelength λ nm (see FIG. 1A).

$$[ Formula 3 ]$$

$$0.5 \times 0.25 \times λ \leq ( d 2 + \frac{t}{2} ) \times n 11 \leq 1.5 \times 0.25 \times λ \qquad ( 2 )$$

Note that in the spectrum of light emitted from the light-emitting device 150, the wavelength λ1 nm, at which the maximum peak is observed, can be used as the wavelength λ nm. Alternatively, in the spectrum of photoluminescent light emitted from the layer 111, the wavelength λ2 nm, at which the maximum peak is observed, can be used as the wavelength λ nm. Alternatively, in the spectrum of photoluminescent light emitted from the light-emitting material contained in the layer 111, the wavelength λ3 nm, at which the maximum peak is observed, can be used as the wavelength λ nm.

Accordingly, light emission efficiency can be increased. Consequently, a novel light-emitting device that is highly convenient, useful, or reliable can be provided.

### <<Structure Example 3 of Layer 113>>

In one embodiment of the present invention, the layer 113A is in contact with the layer 111, and the layer 113A has a function of inhibiting transfer of carriers from the layer 111 toward the layer 113B. For example, the layer 113A has a function of inhibiting transfer of holes.

### <<Example 2 of Material CTM11>>

The material CTM11 has an electron-transport property, and the material CTM11 has a HOMO level HOMO3.

### <<Structure Example 5 of Layer 111>>

The layer 111 contains a host material. The host material has a HOMO level HOMO4, and the HOMO level HOMO4 is higher than the HOMO level HOMO3.

Accordingly, transfer of electrons from the layer 111 toward the layer 113B can be inhibited. Alternatively, the probability of recombination of electrons and holes in the layer 111 can be increased. Alternatively, the light emission efficiency can be increased. Alternatively, the reliability can be increased. Consequently, a novel light-emitting device that is highly convenient, useful, or reliable can be provided.

Note that this embodiment can be combined with any of the other embodiments in this specification as appropriate.

### Embodiment 2

In this embodiment, the structure of the light-emitting device 150 of one embodiment of the present invention is described with reference to FIG. 1A.

### <Structure Example of Light-Emitting Device 150>

The light-emitting device 150 described in this embodiment includes the electrode 101, the electrode 102, and the unit 103. The electrode 102 includes a region overlapping with the electrode 101, and the unit 103 includes a region positioned between the electrode 101 and the electrode 102.

### <Structure Example of Unit 103>

The unit 103 includes the layer 111, the layer 112, and the layer 113 (see FIG. 1A).

The layer 111 includes a region positioned between the layer 112 and the layer 113, the layer 112 includes a region positioned between the electrode 101 and the layer 111, and the layer 113 includes a region positioned between the electrode 102 and the layer 111.

For example, a layer selected from functional layers such as a light-emitting layer, a hole-transport layer, an electron-transport layer, and a carrier-blocking layer can be used in the unit 103. Moreover, a layer selected from functional layers such as a hole-injection layer, an electron-injection layer, an exciton-blocking layer, and a charge-generation layer can be used in the unit 103.

### <<Structure Example 1 of Layer 111>>

For example, a light-emitting material or a light-emitting material and a host material can be used for the layer 111. The layer 111 can be referred to as a light-emitting layer. The layer 111 is preferably provided in a region where holes and electrons are recombined. This allows efficient conversion of energy generated by recombination of carriers into light and emission of the light. Furthermore, the layer 111 is preferably provided apart from a metal used for the electrode or the like. In that case, a quenching phenomenon caused by the metal used for the electrode or the like can be inhibited.

For example, a fluorescent substance, a phosphorescent substance, or a substance exhibiting thermally activated delayed fluorescence (TADF) (also referred to as a TADF material) can be used as the light-emitting material. Thus, energy generated by recombination of carriers can be released as the light EL1 from the light-emitting material (see FIG. 1A).

### [Fluorescent Substance]

A fluorescent substance can be used for the layer 111. For example, the following fluorescent substances can be used for the layer 111. Note that without being limited to the following ones, a variety of known phosphorescent substances can be used for the layer 111.

Specifically, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPrn), N,N-bis(3-methylphenyl)-N,N-bis[3-(9-phenyl-9H-fluoren-9-yl)phenyl]pyrene-1,6-diamine (abbreviation: 1,6mMemFLPAPrn), 1N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N, 9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis[N,N,N-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC 1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PC ABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N,N-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6 [2-(2,3,6,7-tetrahydro-1H, 5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H, 5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahy dro-1H, 5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-m ethoxy-1,1,7,7-tetramethyl-2,3,6,7-tetrahy dro-1H, 5H-benzo[ij]quinolizin-9-yl)ethenyl]-4H-pyran-4-ylidene}propanedinitrile (abbreviation: BisDCJTM), N,N-(pyrene-1,6-diyl)bis[(6,N-diphenylbenzo[b]naphtho[1,2-d]furan)-8-amine] (abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), or 3,10-bis[N-(dibenzofuran-3-yl)-N-phenylamino]naphtho[2,3-b; 6,7-b′]bisbenzofuran (abbreviation: 3,10FrA2Nbf(IV)-02).

Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high light emission efficiency, or high reliability.

### [Phosphorescent Substance]

A phosphorescent substance can be used for the layer 111. For example, the following phosphorescent substances can be used for the layer 111. Note that without being limited to the following ones, a variety of known phosphorescent substances can be used for the layer 111.

For example, any of the following can be used for the layer 111: an organometallic iridium complex having a 4H-triazole skeleton, an organometallic iridium complex having a 1H-triazole skeleton, an organometallic iridium complex having an imidazole skeleton, an organometallic iridium complex having a phenylpyridine derivative with an electron-withdrawing group as a ligand, an organometallic iridium complex having a pyrimidine skeleton, an organometallic iridium complex having a pyrazine skeleton, an organometallic iridium complex having a pyridine skeleton, a rare earth metal complex, and a platinum complex.

### [Phosphorescent Substance (Blue)]

As an organometallic iridium complex having a 4H-triazole skeleton or the like, tris{2-[5-(2-methylphenyl)-4-(2,6-dimethylphenyl)-4H-1,2,4-triazol-3-yl-κN²]phenyl-κC}niridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), or the like can be used.

As an organometallic iridium complex having a 1H-triazole skeleton or the like, tris[3-methyl-1-(2-methylphenyl)-5-phenyl-1H-1,2,4-triazolato]iridium(III) (abbreviation: [Ir(Mptz1-mp)₃]), tris(1-methyl-5-phenyl-3-propyl-1H-1,2,4-triazolato]iridium(III) (abbreviation: [Ir(Prptzl-Me)₃]), or the like can be used.

As an organometallic iridium complex having an imidazole skeleton or the like, fac-tris[1-(2,6-diisopropylphenyl)-2-phenyl-1H-imidazole]iridium(III) (abbreviation: [Ir(iPrpmi)₃]), tris[3-(2,6-dimethylphenyl)-7-methylimidazo[1,2-f]phenanthridinato]iridium(III) (abbreviation: [Ir(dmpimpt-Me)₃]), or the like can be used.

As an organometallic iridium complex having a phenylpyridine derivative with an electron-withdrawing group as a ligand, or the like, bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: Ir(CF₃ppy)₂(pic)), bis[2-(4′,6′-difluorophenyl)pyridinato-N,C^{2′}]iridium(III) acetylacetonate (abbreviation: FIracac), or the like can be used.

Note that these are compounds exhibiting blue phosphorescence, and are compounds having an emission wavelength peak at 440 nm to 520 nm.

### [Phosphorescent Substance (Green)]

As an organometallic iridium complex having a pyrimidine skeleton or the like, it is possible to use, for example, tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), or (acetylacetonato)bis(4,6-diphenylpyrimidinato)iridium(III) (abbreviation: [Ir(dppm)₂(acac)]).

As an organometallic iridium complex having a pyrazine skeleton or the like, (acetylacetonato)bis(3,5-dimethyl-2-phenylpyrazinato)iridium(III) (abbreviation: [Ir(mppr-Me)₂(acac)]), (acetylacetonato)bis(5-isopropyl-3-methyl-2-phenylpyrazinato)iridium(III) (abbreviation: [Ir(mppr-iPr)₂(acac)]), or the like can be used.

As an organometallic iridium complex having a pyridine skeleton or the like, it is possible to use, for example, tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N, C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d3-methyl-(2-pyridinyl-κN)benzofuro[2,3-b]pyridine-κC]bis[2-(5-d3-methyl-2-pyridyl-κN2)phenyl-κ]iridium(III) (abbreviation: [Ir(5mppy-d3)₂(mbfpypy-d3)]), or [2-d3-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy-d3)]).

An example of a rare earth metal complex is tris(acetylacetonato) (monophenanthroline)terbium(III) (abbreviation: [Tb(acac)₃(Phen)]).

Note that these are compounds mainly exhibiting green phosphorescence, and have an emission wavelength peak at 500 nm to 600 nm. An organometallic iridium complex having a pyrimidine skeleton excels particularly in reliability or light emission efficiency.

### [Phosphorescent Substance (Red)]

As an organometallic iridium complex having a pyrimidine skeleton or the like, (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), bis[4,6-di(naphthalen-1-yl)pyrimidinato] (dipivaloylmethanato)iridium(III) (abbreviation: [Ir(d1npm)₂(dpm)]), or the like can be used.

As an organometallic iridium complex having a pyrazine skeleton or the like, (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), (acetylacetonato)bis[2,3-bis(4-fluorophenyl)quinoxalinato]iridium(III) (abbreviation: [Ir(Fdpq)₂(acac)]), or the like can be used.

As an organometallic iridium complex having a pyridine skeleton or the like, tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), or the like can be used.

As a rare earth metal complex or the like, tris(1,3-diphenyl-1,3-propanedionato)(monophenanthroline)europium(III) (abbreviation: [Eu(DBM)₃(Phen)]), tris[1-(2-thenoyl)-3,3,3-trifluoroacetonato] (monophenanthroline)europium(III) (abbreviation: [Eu(TTA)₃(Phen)]), or the like can be used.

As a platinum complex or the like, 2,3,7,8,12,13,17,18-octaethyl-21H,23H-porphyrin platinum(II) (abbreviation: PtOEP) or the like can be used.

Note that these are compounds exhibiting red phosphorescence, and have an emission peak at 600 nm to 700 nm. Furthermore, from the organometallic iridium complex having a pyrazine skeleton, red light emission with chromaticity favorably used for display apparatuses can be obtained.

### [Substance Exhibiting Thermally Activated Delayed Fluorescence (TADF)]

A TADF material can be used for the layer 111. For example, any of the TADF materials given below can be used as the light-emitting material. Note that without being limited thereto, a variety of known TADF materials can be used as the light-emitting material.

In the TADF material, the difference between the S1 level and the T1 level is small, and reverse intersystem crossing (upconversion) from the triplet excited state into the singlet excited state can be achieved by a little thermal energy. Thus, the singlet excited state can be efficiently generated from the triplet excited state. In addition, the triplet excitation energy can be converted into light.

An exciplex whose excited state is formed of two kinds of substances has an extremely small difference between the S1 level and the T1 level and functions as a TADF material capable of converting triplet excitation energy into singlet excitation energy.

A phosphorescent spectrum observed at a low temperature (e.g., 77 K to 10 K) is used for an index of the T1 level. When the level of energy with a wavelength of the line obtained by extrapolating a tangent to the fluorescent spectrum at a tail on the short wavelength side is the S level and the level of energy with a wavelength of the line obtained by extrapolating a tangent to the phosphorescent spectrum at a tail on the short wavelength side is the T1 level, the difference between S1 and T1 of the TADF material is preferably smaller than or equal to 0.3 eV, further preferably smaller than or equal to 0.2 eV.

When a TADF material is used as the light-emitting substance, the S1 level of the host material is preferably higher than the S1 level of the TADF material. In addition, the T1 level of the host material is preferably higher than the T1 level of the TADF material.

For example, a fullerene, a derivative thereof, an acridine, a derivative thereof, an eosin derivative, or the like can be used as the TADF material. Furthermore, porphyrin containing a metal such as magnesium (Mg), zinc (Zn), cadmium (Cd), tin (Sn), platinum (Pt), indium (In), or palladium (Pd) can be used as the TADF material.

Specifically, any of the following materials whose structural formulae are shown below can be used: a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex SnF₂(Etio I)), an octaethylporphyrin-platinum chloride complex (PtCl₂OEP), and the like.

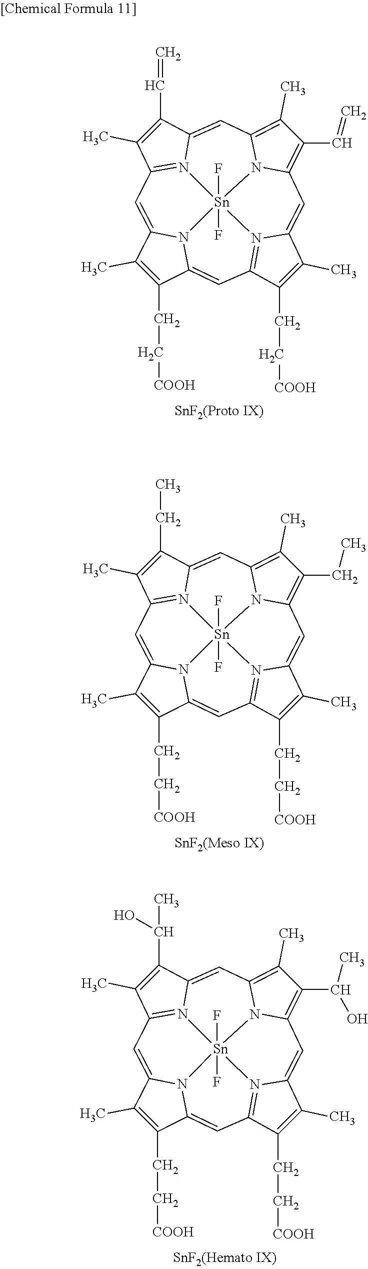

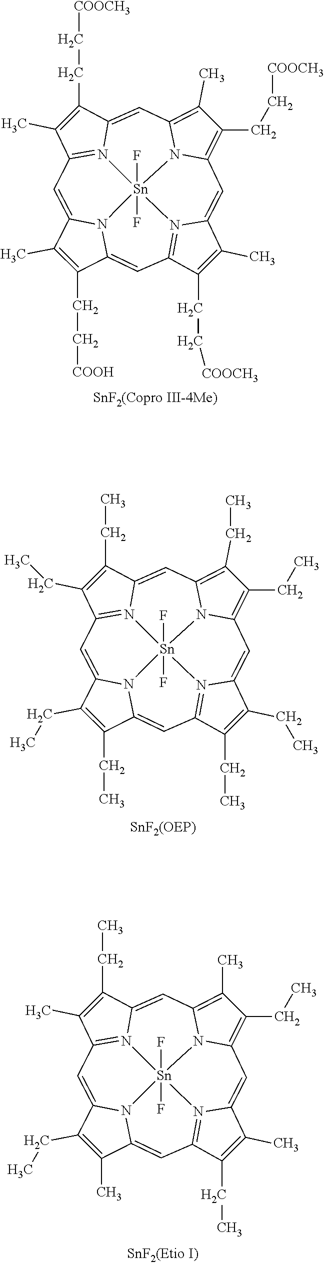

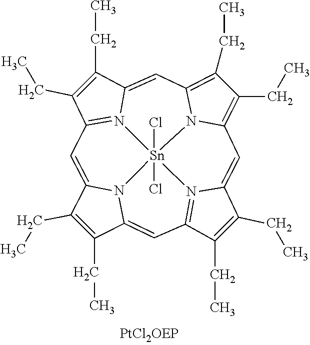

Furthermore, a heterocyclic compound including one or both of a π-electron rich heteroaromatic ring and a π-electron deficient heteroaromatic ring can be used, for example, as the TADF material.

Specifically, any of the following materials whose structural formulae are shown below can be used, for example: 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazine-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), and 10-phenyl-10H,10′H-spiro[acridin-9,9′-anthracen]-10′-one (abbreviation: ACRSA).

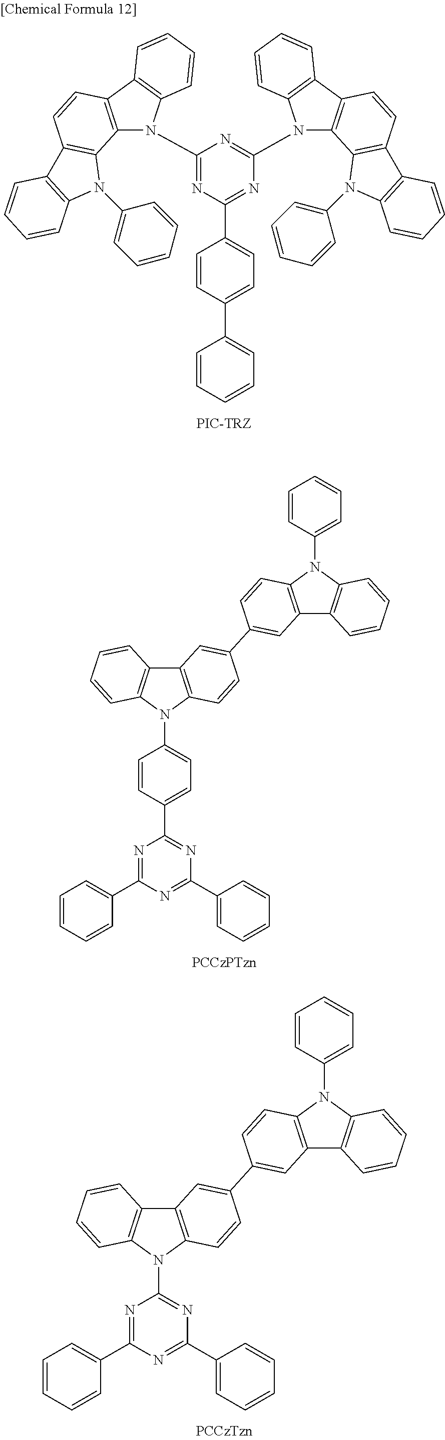 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

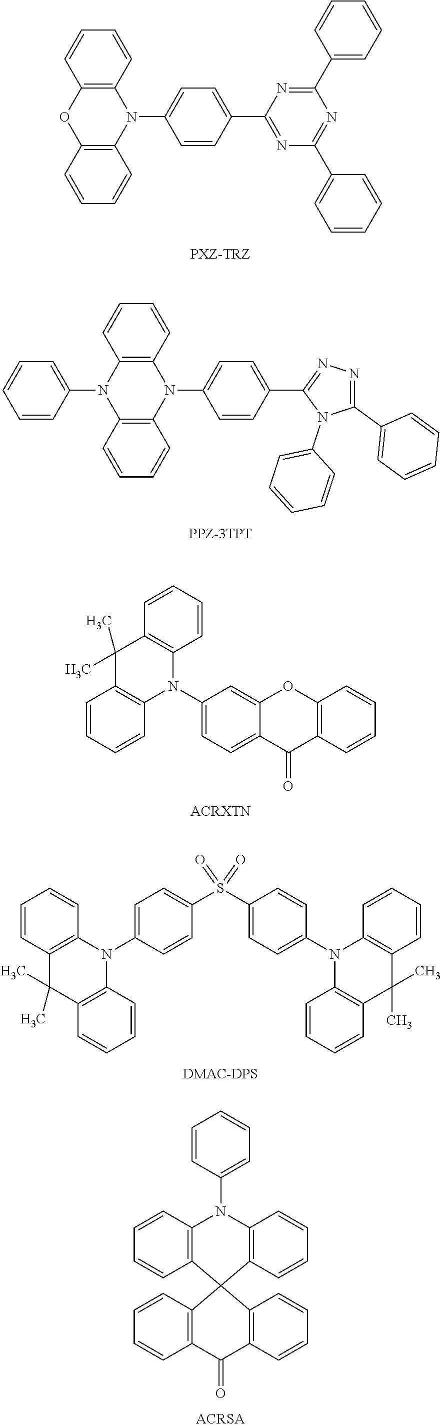 [SMILES: c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

Such a heterocyclic compound is preferable because of having high electron-transport and hole-transport properties owing to a π-electron rich heteroaromatic ring and a π-electron deficient heteroaromatic ring. In particular, among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferred because of their stability and favorable reliability. In particular, a benzofuropyrimidine skeleton, a benzothienopyrimidine skeleton, a benzofuropyrazine skeleton, and a benzothienopyrazine skeleton are preferred because of their high acceptor properties and favorable reliability.

Among skeletons having the π-electron rich heteroaromatic ring, an acridine skeleton, a phenoxazine skeleton, a phenothiazine skeleton, a furan skeleton, a thiophene skeleton, and a pyrrole skeleton have stability and favorable reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable.

Note that a substance in which the π-electron rich heteroaromatic ring is directly bonded to the π-electron deficient heteroaromatic ring is particularly preferred because the electron-donating property of the π-electron rich heteroaromatic ring and the electron-accepting property of the π-electron deficient heteroaromatic ring are both improved, the energy difference between the S1 level and the T1 level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used.

As a π-electron deficient skeleton, a xanthene skeleton, a thioxanthene dioxide skeleton, an oxadiazole skeleton, a triazole skeleton, an imidazole skeleton, an anthraquinone skeleton, a boron-containing skeleton such as phenylborane or boranthrene, an aromatic ring or a heteroaromatic ring having a nitrile group or a cyano group, such as benzonitrile or cyanobenzene, a carbonyl skeleton such as benzophenone, a phosphine oxide skeleton, a sulfone skeleton, or the like can be used.

As described above, a π-electron deficient skeleton and a π-electron rich skeleton can be used instead of at least one of the π-electron deficient heteroaromatic ring and the π-electron rich heteroaromatic ring.

### <<Structure Example 2 of Layer 111>>

A material having a carrier-transport property can be used as the host material. For example, a material having a hole-transport property, a material having an electron-transport property, a substance exhibiting thermally activated delayed fluorescence (TADF), a material having an anthracene skeleton, or a mixed material can be used as the host material.

### [Material Having Hole-Transport Property]

A material having a hole mobility of 1×10⁻⁶ cm²/Vs or higher can be suitably used as the material having a hole-transport property.

For example, a material having a hole-transport property that can be used for the layer 112 can be used for the layer 111. Specifically, a material having a hole-transport property that can be used for the hole-transport layer can be used for the layer 111.

### [Material Having Electron-Transport Property]

For example, a material having an electron-transport property that can be used for the layer 113 can be used for the layer 111. Specifically, a material having an electron-transport property that can be used for the electron-transport layer can be used for the layer 111.

### [Material Having Anthracene Skeleton]

An organic compound having an anthracene skeleton can be used as the host material. An organic compound having an anthracene skeleton is preferable particularly in the case where a fluorescent substance is used as the light-emitting substance. Thus, a light-emitting device with high emission efficiency and high durability can be obtained.

As the organic compound having an anthracene skeleton, an organic compound having a diphenylanthracene skeleton, in particular, a 9,10-diphenylanthracene skeleton is chemically stable and thus is preferable. The host material preferably has a carbazole skeleton, in which case the hole-injection and hole-transport properties are improved. In particular, the host material preferably has a dibenzocarbazole skeleton, in which case the HOMO level thereof is shallower than that of carbazole by approximately 0.1 eV so that holes enter the host material easily, the hole-transport property is improved, and the heat resistance is increased. Note that in terms of the hole-injection and hole-transport properties, a benzofluorene skeleton or a dibenzofluorene skeleton may be used instead of a carbazole skeleton.

Thus, a substance having both a 9,10-diphenylanthracene skeleton and a carbazole skeleton, a substance having both a 9,10-diphenylanthracene skeleton and a benzocarbazole skeleton, or a substance having both a 9,10-diphenylanthracene skeleton and a dibenzocarbazole skeleton is preferable as the host material.

For example, it is possible to use 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), 9-(1-naphthyl)-10-[4-(2-naphthyl)phenyl]anthracene (abbreviation: aN-(3NPAnth), 9-phenyl-3-[4-(10-phenyl-9-anthryl)phenyl]-9H-carbazole (abbreviation: PCzPA), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), or 3-[4-(1-naphthyl)-phenyl]-9-phenyl-9H-carbazole (abbreviation: PCPN).

In particular, CzPA, cgDBCzPA, 2mBnfPPA, and PCzPA have excellent characteristics.

### [Substance Exhibiting Thermally Activated Delayed Fluorescence (TADF)]

A TADF material can be used for the layer 111. For example, any of the TADF materials given below can be used as the host material. Note that without being limited thereto, a variety of known TADF materials can be used as the host material.

When the TADF material is used as the host material, triplet excitation energy generated in the TADF material can be converted into singlet excitation energy by reverse intersystem crossing. Moreover, excitation energy can be transferred to the light-emitting substance. In other words, the TADF material functions as an energy donor, and the light-emitting substance functions as an energy acceptor. Thus, the emission efficiency of the light-emitting device can be increased.

This is very effective in the case where the light-emitting substance is a fluorescent substance. In that case, in order to achieve high emission efficiency, the S1 level of the TADF material is preferably higher than the S1 level of the fluorescent substance. Furthermore, the T1 level of the TADF material is preferably higher than the S1 level of the fluorescent substance. Therefore, the T1 level of the TADF material is preferably higher than the T1 level of the fluorescent substance.

It is also preferable to use a TADF material that exhibits light emission overlapping with the wavelength of a lowest-energy-side absorption band of the fluorescent substance. This enables smooth transfer of excitation energy from the TADF material to the fluorescent substance and accordingly enables efficient light emission, which is preferable.

In order that singlet excitation energy is efficiently generated from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton that causes light emission) of the fluorescent substance. As the protective group, a substituent having no n bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituent having no n bond has a poor carrier-transport property; thus, the TADF material and the luminophore of the fluorescent substance can be made away from each other with little influence on carrier transportation or carrier recombination.

Here, the luminophore refers to an atomic group (skeleton) that causes light emission in a fluorescent substance. The luminophore is preferably a skeleton having a n bond, further preferably includes an aromatic ring, and still further preferably includes a condensed aromatic ring or a condensed heteroaromatic ring.

Examples of the condensed aromatic ring or the condensed heteroaromatic ring include a phenanthrene skeleton, a stilbene skeleton, an acridone skeleton, a phenoxazine skeleton, and a phenothiazine skeleton. Specifically, fluorescent substances having a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton are preferable because of their high fluorescence quantum yields.

For example, the TADF material that can be used as the light-emitting material can be used as the host material.

### [Structure Example 1 of Mixed Material]

A material in which a plurality of kinds of substances are mixed can be used as the host material. For example, a material having an electron-transport property and a material having a hole-transport property can be used in the mixed material. The weight ratio of the material having a hole-transport property to the material having an electron-transport property in the mixed material is the material having a hole-transport property: the material having an electron-transport property=1:19 to 19:1. Accordingly, the carrier-transport property of the layer 111 can be easily adjusted. A recombination region can also be easily controlled.

### [Structure Example 2 of Mixed Material]

A material mixed with a phosphorescent substance can be used as the host material. When a fluorescent substance is used as the light-emitting substance, a phosphorescent substance can be used as an energy donor for supplying excitation energy to the fluorescent substance.

A mixed material containing a material to form an exciplex can be used as the host material. For example, a material in which an emission spectrum of a formed exciplex overlaps with a wavelength of the absorption band on the lowest energy side of the light-emitting substance can be used as the host material. This enables smooth energy transfer and improves emission efficiency. Alternatively, the driving voltage can be lowered.

A phosphorescent substance can be used as at least one of the materials forming an exciplex. Accordingly, reverse intersystem crossing can be used. Alternatively, triplet excitation energy can be efficiently converted into singlet excitation energy.

A combination of materials forming an exciplex is preferably such that the HOMO level of a material having a hole-transport property is higher than or equal to the HOMO level of a material having an electron-transport property. Alternatively, the LUMO level of the material having a hole-transport property is preferably higher than or equal to the LUMO level of the material having an electron-transport property. Thus, an exciplex can be efficiently formed. Note that the LUMO levels and the HOMO levels of the materials can be derived from the electrochemical characteristics (reduction potentials and oxidation potentials). Specifically, the reduction potentials and the oxidation potentials can be measured by cyclic voltammetry (CV).

The formation of an exciplex can be confirmed by a phenomenon in which the emission spectrum of a mixed film in which the material having a hole-transport property and the material having an electron-transport property are mixed is shifted to the longer wavelength side than the emission spectrum of each of the materials (or has another peak on the longer wavelength side) observed by comparison of the emission spectrum of the material having a hole-transport property, the emission spectrum of the material having an electron-transport property, and the emission spectrum of the mixed film of these materials, for example. Alternatively, the formation of an exciplex can be confirmed by a difference in transient response, such as a phenomenon in which the transient PL lifetime of the mixed film has longer lifetime components or has a larger proportion of delayed components than that of each of the materials, observed by comparison of transient photoluminescence (PL) of the material having a hole-transport property, the transient PL of the material having an electron-transport property, and the transient PL of the mixed film of these materials. The transient PL can be rephrased as transient electroluminescence (EL). That is, the formation of an exciplex can also be confirmed by a difference in transient response observed by comparison of the transient EL of the material having a hole-transport property, the transient EL of the material having an electron-transport property, and the transient EL of the mixed film of these materials.

Note that this embodiment can be combined with any of the other embodiments in this specification as appropriate.

### Embodiment 3

In this embodiment, the structure of the light-emitting device 150 of one embodiment of the present invention is described with reference to FIG. 1A.

### <Structure Example of Light-Emitting Device 150>

The light-emitting device 150 described in this embodiment includes the electrode 101, the electrode 102, the unit 103, and the layer 104. The electrode 102 includes a region overlapping with the electrode 101, and the unit 103 includes a region positioned between the electrode 101 and the electrode 102. The layer 104 includes a region positioned between the electrode 101 and the unit 103. For example, any of the structures described in Embodiment 1 and Embodiment 2 can be used for the unit 103.

### <Structure Example of Electrode 101>

For example, a conductive material can be used for the electrode 101. Specifically, a metal, an alloy, a conductive compound, a mixture of these, or the like can be used for the electrode 101. For example, a material having a work function higher than or equal to 4.0 eV can be suitably used.

For example, indium oxide-tin oxide (ITO: Indium Tin Oxide), indium oxide-tin oxide containing silicon or silicon oxide, indium oxide-zinc oxide, indium oxide containing tungsten oxide and zinc oxide (IWZO), or the like can be used.

Furthermore, for example, gold (Au), platinum (Pt), nickel (Ni), tungsten (W), chromium (Cr), molybdenum (Mo), iron (Fe), cobalt (Co), copper (Cu), palladium (Pd), a nitride of a metal material (e.g., titanium nitride), or the like can be used. Alternatively, graphene can be used.

### <<Structure Example of Layer 104>>

For example, a material having a hole-injection property can be used for the layer 104. The layer 104 can be referred to as a hole-injection layer.

Specifically, a substance having an acceptor property can be used for the layer 104. Alternatively, a material in which a substance having an acceptor property and a material having a hole-transport property are combined can be used for the layer 104. This can facilitate injection of holes from the electrode 101, for example. Alternatively, the driving voltage of the light-emitting device can be lowered.

### [Substance Having Acceptor Property]

An organic compound and an inorganic compound can be used as the substance having an acceptor property. The substance having an acceptor property can extract electrons from an adjacent hole-transport layer or an adjacent material having a hole-transport property by the application of an electric field.

For example, a compound having an electron-withdrawing group (a halogen group or a cyano group) can be used as the substance having an acceptor property. Note that an organic compound having an acceptor property is easily evaporated and deposited. As a result, the productivity of the light-emitting device can be increased.

Specifically, it is possible to use, for example, 7,7,8,8-tetracyano-2,3,5,6-tetrafluoroquinodimethane (abbreviation: F₄-TCNQ), chloranil, 2,3,6,7,10,11-hexacy ano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene)malononitrile.

A compound in which electron-withdrawing groups are bonded to a condensed aromatic ring having a plurality of heteroatoms, such as HAT-CN, is particularly preferable because it is thermally stable.

Alternatively, a [3]radialene derivative having an electron-withdrawing group (in particular, a cyano group or a halogen group such as a fluoro group) is preferable because it has a very high electron-accepting property.

Specifically, it is possible to use, for example, α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], or α,α′,α″-1,2,3-cyclopropanetriylidenetris[2,3,4,5,6-pentafluorobenzeneacetonitrile]

As the substance having an acceptor property, a molybdenum oxide, a vanadium oxide, a ruthenium oxide, a tungsten oxide, a manganese oxide, or the like can be used.

Alternatively, it is possible to use any of the following compounds: phthalocyanine-based complex compounds such as phthalocyanine (abbreviation: H₂Pc) and copper phthalocyanine (CuPc); and compounds having an aromatic amine skeleton, such as 4,4′-bis[N-(4-diphenylaminophenyl)-N-phenylamino]biphenyl (abbreviation: DPAB) and N,N′-bis{4-[bis(3-methylphenyl)amino]phenyl}-N,N′-diphenyl-(1,1′-biphenyl)-4,4′-diamine (abbreviation: DNTPD).

Alternatively, a high molecular compound such as poly(3,4-ethylenedioxythiophene)/poly(styrenesulfonic acid) (PEDOT/PSS), or the like can be used.

### [Structure Example 1 of Composite Material]

A material in which a plurality of kinds of substances are combined can be used as the material having a hole-injection property. For example, a substance having an acceptor property and a material having a hole-transport property can be used for the composite material. Thus, besides a material having a high work function, a material having a low work function can also be used for the electrode 101. Alternatively, a material used for the electrode 101 can be selected from a wide range of materials regardless of its work function.

As the material having a hole-transport property in the composite material, for example, a compound having an aromatic amine skeleton, a carbazole derivative, an aromatic hydrocarbon, an aromatic hydrocarbon having a vinyl group, a high molecular compound (such as an oligomer, a dendrimer, or a polymer), or the like can be used. A material having a hole mobility of 1×10⁻⁶ cm²/Vs or higher can be suitably used as the material having a hole-transport property in the composite material.

A substance having a relatively deep HOMO level can be suitably used as the material having a hole-transport property in the composite material. Specifically, the HOMO level is preferably higher than or equal to −5.7 eV and lower than or equal to −5.4 eV, in which case hole injection to the unit 103 can be facilitated. Alternatively, hole injection to the layer 112 can be facilitated. Alternatively, the reliability of the light-emitting device can be increased.

As the compound having an aromatic amine skeleton, for example, N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), or the like can be used.

As the carbazole derivative, for example, 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(N-carbazolyl)]phenyl-10-phenylanthracene (abbreviation: CzPA), 1,4-bis[4-(N-carbazolyl)phenyl]-2,3,5,6-tetraphenylbenzene, or the like can be used.

As the aromatic hydrocarbon, for example, 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, 2,5,8,11-tetra(tert-butyl)perylene, pentacene, coronene, or the like can be used.

As the aromatic hydrocarbon having a vinyl group, for example, 4,4′-bis(2,2-diphenylvinyl)biphenyl (abbreviation: DPVBi), 9,10-bis[4-(2,2-diphenylvinyl)phenyl]anthracene (abbreviation: DPVPA), or the like can be used.

As the high molecular compound, for example, poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), poly[N,N′-bis(4-butylphenyl)-N,N′-bis(phenyl)benzidine] (abbreviation: Poly-TPD), or the like can be used.

As another example, a substance having any of a carbazole skeleton, a dibenzofuran skeleton, a dibenzothiophene skeleton, and an anthracene skeleton can be favorably used as the material having a hole-transport property in the composite material. Moreover, as the material having a hole-transport property in the composite material, it is possible to use a substance including any of an aromatic amine having a substituent that includes a dibenzofuran ring or a dibenzothiophene ring, an aromatic monoamine that includes a naphthalene ring, and an aromatic monoamine in which a 9-fluorenyl group is bonded to nitrogen of amine through an arylene group. With the use of a substance including an N,N-bis(4-biphenyl)amino group, the reliability of the light-emitting device can be increased.

As such a material, it is possible to use, for example, N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnffiB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBMB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yl)triphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenyl amine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-diphenyl-4′-(2-naphthyl)-4″-{9-(4-biphenylyl)carbazole)}triphenylamine (abbreviation: YGTBi(3NB), N-[4-(9-phenyl-9H-carbazol-3-yl)phenyl]-N-[4-(1-naphthyl)phenyl]-9,9′-spirobi (9H-fluoren)-2-amine (abbreviation: PCBNBSF), N,N-bis(4-biphenylyl)-9,9′-spirobi[9H-fluoren]-2-amine (abbreviation: BBASF), N,N-bis(1,1′-biphenyl-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi(9H-fluoren)-4-amine (abbreviation: oFBiSF), N-(4-biphenyl)-N-(dibenzofuran-4-yl)-9,9-dimethyl-9H-fluoren-2-amine (abbreviation: FrBiF), N-[4-(1-naphthyl)phenyl]-N-[3-(6-phenyl dibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]spiro-9,9′-bifluoren-2-amine (abbreviation: PCBASF), N-(1,1′-biphenyl-4-yl)-9,9-dimethyl-N-[4-(9-phenyl-9H-carbazol-3-yl)phenyl]-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, or N,N-bis(9,9-dimethyl-9H-fluoren-2-yl)-9,9′-spirobi-9H-fluoren-1-amine.

### [Structure Example 2 of Composite Material]

For example, a composite material including a material having an acceptor property, a material having a hole-transport property, and a fluoride of an alkali metal or a fluoride of an alkaline earth metal can be used as the material having a hole-injection property. In particular, a composite material in which the proportion of fluorine atoms is higher than or equal to 20% can be favorably used. Thus, the refractive index of the layer 104 can be reduced. Alternatively, a layer with a low refractive index can be formed inside the light-emitting device. Alternatively, the external quantum efficiency of the light-emitting device can be improved.

Note that this embodiment can be combined with any of the other embodiments in this specification as appropriate.

### Embodiment 4

In this embodiment, the structure of the light-emitting device 150 of one embodiment of the present invention is described with reference to FIG. 1A.

### <Structure Example of Light-Emitting Device 150>

The light-emitting device 150 described in this embodiment includes the electrode 101, the electrode 102, the unit 103, and a layer 105. The electrode 102 includes a region overlapping with the electrode 101, and the unit 103 includes a region positioned between the electrode 101 and the electrode 102. The layer 105 includes a region positioned between the unit 103 and the electrode 102. For example, the structure described in any of Embodiment 1 to Embodiment 3 can be used for the unit 103.

### <Structure Example of Electrode 102>

A conductive material can be used for the electrode 102, for example. Specifically, a metal, an alloy, a conductive compound, a mixture of these, or the like can be used for the electrode 102. For example, a material having a lower work function than the electrode 101 can be suitably used for the electrode 102. Specifically, a material having a work function lower than or equal to 3.8 eV is preferable.

For example, an element belonging to Group 1 of the periodic table, an element belonging to Group 2 of the periodic table, a rare earth metal, or an alloy containing any of these elements can be used for the electrode 102.

Specifically, lithium (Li), cesium (Cs), or the like; magnesium (Mg), calcium (Ca), strontium (Sr), or the like; europium (Eu), ytterbium (Yb), or the like; or an alloy containing any of these (MgAg or AlLi) can be used for the electrode 102.

### <<Structure Example of Layer 105>>

A material having an electron-injection property can be used for the layer 105, for example. The layer 105 can be referred to as an electron-injection layer.

Specifically, a substance having a donor property can be used for the layer 105. Alternatively, a material in which a substance having a donor property and a material having an electron-transport property are combined can be used for the layer 105. Alternatively, electride can be used for the layer 105. This can facilitate injection of electrons from the electrode 102, for example. Alternatively, besides a material having a low work function, a material having a high work function can also be used for the electrode 102. Alternatively, a material used for the electrode 102 can be selected from a wide range of materials regardless of its work function. Specifically, Al, Ag, ITO, indium oxide-tin oxide containing silicon or silicon oxide, or the like can be used for the electrode 102. Alternatively, the driving voltage of the light-emitting device can be lowered.

### [Substance Having Donor Property]

For example, an alkali metal, an alkaline earth metal, a rare earth metal, or a compound thereof (an oxide, a halide, a carbonate, or the like) can be used as the substance having a donor property. Alternatively, an organic compound such as tetrathianaphthacene (abbreviation: TTN), nickelocene, or decamethylnickelocene can be used as the substance having a donor property.

As an alkali metal compound (including an oxide, a halide, and a carbonate), lithium oxide, lithium fluoride (LiF), cesium fluoride (CsF), lithium carbonate, cesium carbonate, 8-hydroxyquinolinato-lithium (abbreviation: Liq), or the like can be used.

As an alkaline earth metal compound (including an oxide, a halide, and a carbonate), calcium fluoride (CaF₂) or the like can be used.

### [Structure Example of Composite Material]

A material in which a plurality of kinds of substances are combined can be used as the material having an electron-injection property. For example, a substance having a donor property and a material having an electron-transport property can be used for the composite material. As another example, a material having an electron-transport property usable for the unit 103 can be used for the composite material.

A material including a fluoride of an alkali metal in a microcrystalline state and a material having an electron-transport property can be used for the composite material. Alternatively, a material including a fluoride of an alkaline earth metal in a microcrystalline state and a material having an electron-transport property can be used for the composite material. In particular, a composite material including a fluoride of an alkali metal or a fluoride of an alkaline earth metal at 50 wt % or higher can be suitably used. Alternatively, a composite material including an organic compound having a bipyridine skeleton can be suitably used. Thus, the refractive index of the layer 104 can be reduced. Alternatively, the external quantum efficiency of the light-emitting device can be improved.

### [Electride]

For example, a substance obtained by adding electrons at high concentration to an oxide where calcium and aluminum are mixed, or the like can be used as the material having an electron-injection property.

Note that this embodiment can be combined with any of the other embodiments in this specification as appropriate.

### Embodiment 5

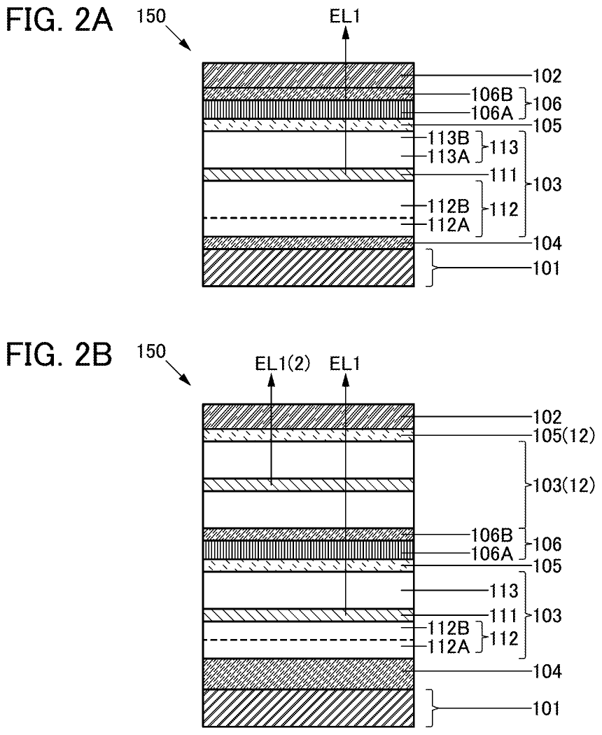

In this embodiment, the structure of the light-emitting device 150 of one embodiment of the present invention is described with reference to FIG. 2A.

FIG. 2A is a cross-sectional view illustrating a structure of the light-emitting device of one embodiment of the present invention.

### <Structure Example of Light-Emitting Device 150>

The light-emitting device 150 described in this embodiment includes the electrode 101, the electrode 102, the unit 103, and an intermediate layer 106 (see FIG. 2A). The electrode 102 includes a region overlapping with the electrode 101, and the unit 103 includes a region positioned between the electrode 101 and the electrode 102. The intermediate layer 106 includes a region positioned between the unit 103 and the electrode 102.

### <<Structure Example of Intermediate Layer 106>>

The intermediate layer 106 includes a layer 106A and a layer 106B. The layer 106B includes a region positioned between the layer 106A and the electrode 102.

### <<Structure Example of Layer 106A>>

For example, a material having an electron-transport property can be used for the layer 106A. The layer 106A can be referred to as an electron-relay layer. With the use of the layer 106A, a layer that is in contact with the anode side of the layer 106A can be distanced from a layer that is in contact with the cathode side of the layer 106A. It is possible to reduce interaction between the layer in contact with the anode side of the layer 106A and the layer in contact with the cathode side of the layer 106A. Electrons can be smoothly supplied to the layer in contact with the anode side of the layer 106A.

A substance whose LUMO level is positioned between the LUMO level of the substance having an acceptor property included in the layer in contact with the anode side of the layer 106A and the LUMO level of the substance included in the layer in contact with the cathode side of the layer 106A can be suitably used for the layer 106A.

For example, a material that has a LUMO level in a range higher than or equal to −5.0 eV, preferably higher than or equal to −5.0 eV and lower than or equal to −3.0 eV can be used for the layer 106A.

Specifically, a phthalocyanine-based material can be used for the layer 106A. Alternatively, a metal complex having a metal-oxygen bond and an aromatic ligand can be used for the layer 106A.

### <<Structure Example of Layer 106B>>

For example, a material that supplies electrons to the anode side and supplies holes to the cathode side when voltage is applied can be used for the layer 106B. Specifically, electrons can be supplied to the unit 103 that is positioned on the anode side. The layer 106B can be referred to as a charge-generation layer.

Specifically, a material having a hole-injection property usable for the layer 104 can be used for the layer 106B. For example, a composite material can be used for the layer 106B. As another example, a stacked film in which a film including the composite material and a film including a material having a hole-transport property are stacked can be used as the layer 106B.

Note that this embodiment can be combined with any of the other embodiments in this specification as appropriate.

### Embodiment 6

In this embodiment, the structure of the light-emitting device 150 of one embodiment of the present invention is described with reference to FIG. 2B.

FIG. 2B is a cross-sectional view illustrating a structure of the light-emitting device of one embodiment of the present invention, which is different from the structure illustrated in FIG. 2A.

### <Structure Example of Light-Emitting Device 150>

The light-emitting device 150 described in this embodiment includes the electrode 101, the electrode 102, the unit 103, the intermediate layer 106, and a unit 103(12) (see FIG. 2B). The 30 electrode 102 includes a region overlapping with the electrode 101, the unit 103 includes a region positioned between the electrode 101 and the electrode 102, and the intermediate layer 106 includes a region positioned between the unit 103 and the electrode 102. The unit 103(12) includes a region positioned between the intermediate layer 106 and the electrode 102.

A structure including the intermediate layer 106 and a plurality of units is referred to as a stacked light-emitting device or a tandem light-emitting device in some cases. This structure can obtain light emission at high luminance while the current density is kept low. Alternatively, the reliability can be increased. Alternatively, the driving voltage can be lowered compared to other structures with the same luminance. Alternatively, power consumption can be reduced.

### <<Structure Example of Unit 103(12)>>

The structure usable for the unit 103 can be employed for the unit 103(12). In other words, the light-emitting device 150 includes a plurality of units that are stacked. Note that the number of stacked units is not limited to two, and three or more units can be stacked.

The same structure as the unit 103 can be employed for the unit 103(12). Alternatively, a structure different from that of the unit 103 can be employed for the unit 103(12).

For example, a structure that exhibits a different emission color from the emission color of the unit 103 can be employed for the unit 103(12). Specifically, the unit 103 that emits red light and green light and the unit 103(12) that emits blue light can be employed. Accordingly, a light-emitting device that emits light of a desired color can be provided. For example, a light-emitting device that emits white light can be provided.

### <<Structure Example of Intermediate Layer 106>>

The intermediate layer 106 has a function of supplying electrons to one of the unit 103 and the unit 103(12) and supplying holes to the other. For example, the intermediate layer 106 described in Embodiment 5 can be used.

### <Manufacturing Method of Light-Emitting Device 150>

For example, each layer of the electrode 101, the electrode 102, the unit 103, the intermediate layer 106, and the unit 103(12) can be formed by a dry process, a wet process, an evaporation method, a droplet discharge method, a coating method, a printing method, or the like. Different methods can be used to form the components.

Specifically, the light-emitting device 150 can be manufactured with a vacuum evaporation machine, an inkjet machine, a coating machine such as a spin coater, a gravure printing machine, an offset printing machine, a screen printing machine, or the like.

For example, the electrode can be formed by a wet process or a sol-gel method using a paste of a metal material. Specifically, an indium oxide-zinc oxide film can be formed by a sputtering method using a target obtained by adding zinc oxide to indium oxide at 1 to 20 wt %. Furthermore, an indium oxide film containing tungsten oxide and zinc oxide (IWZO) can be formed by a sputtering method using a target containing, with respect to indium oxide, tungsten oxide at 0.5 to 5 wt % and zinc oxide at 0.1 to 1 wt %.

Note that this embodiment can be combined with any of the other embodiments in this specification as appropriate.

### Embodiment 7

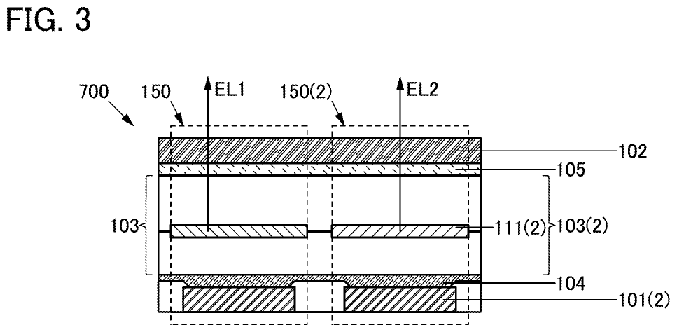

In this embodiment, a structure of a light-emitting panel 700 of one embodiment of the present invention is described with reference to FIG. 3.

### <Structure Example of Light-Emitting Panel 700>

The light-emitting panel 700 described in this embodiment includes the light-emitting device 150 and a light-emitting device 150(2) (FIG. 3).

For example, the light-emitting device described in any of Embodiment 1 to Embodiment 6 can be used as the light-emitting device 150.

### <Structure Example of Light-Emitting Device 150(2)>

The light-emitting device 150(2) described in this embodiment includes an electrode 101(2), the electrode 102, and a unit 103(2) (see FIG. 3). The electrode 102 includes a region overlapping with the electrode 101(2). Note that some of the components of the light-emitting device 150 can be used as some of the components of the light-emitting device 150(2). Thus, some of the components can be used in common. Alternatively, the manufacturing process can be simplified.

### <<Structure Example of Unit 103(2)>>

The unit 103(2) includes a region positioned between the electrode 101(2) and the electrode 102, and the unit 103(2) includes a layer 111(2).

The unit 103(2) has a single-layer structure or a stacked-layer structure. For example, the unit 103(2) can include a layer selected from functional layers such as a hole-transport layer, an electron-transport layer, a carrier-blocking layer, and an exciton-blocking layer.

The unit 103(2) includes a region where electrons injected from one of the electrodes recombine with holes injected from the other electrode. For example, the unit 103(2) includes a region where holes injected from the electrode 101(2) recombine with electrons injected from the electrode 102.

### <<Structure Example 1 of Layer 111(2)>>

The layer 111(2) contains a light-emitting material and a host material. The layer 111(2) can be referred to as a light-emitting layer. The layer 111(2) is preferably provided in a region where holes and electrons are recombined. This allows efficient conversion of energy generated by recombination of carriers into light and emission of the light. Furthermore, the layer 111(2) is preferably provided apart from a metal used for the electrode or the like. In that case, a quenching phenomenon caused by the metal used for the electrode or the like can be inhibited.

For example, a light-emitting material different from the light-emitting material used for the layer 111 can be used for the layer 111(2). Specifically, a light-emitting material having a different emission color from that of the layer 111 can be used for the layer 112(2). Thus, light-emitting devices with different hues can be provided. Alternatively, additive color mixing can be performed using a plurality of light-emitting devices with different hues. Alternatively, it is possible to express a color of a hue that an individual light-emitting device cannot display.

For example, a light-emitting device that emits blue light, a light-emitting device that emits green light, and a light-emitting device that emits red light can be provided in the functional panel. Alternatively, a light-emitting device that emits white light, a light-emitting device that emits yellow light, and a light-emitting device that emits infrared rays can be provided in the functional panel.

Note that this embodiment can be combined with any of the other embodiments in this specification as appropriate.

### Embodiment 8

In this embodiment, a light-emitting apparatus including the light-emitting device described in any one of Embodiment 1 to Embodiment 6 is described.

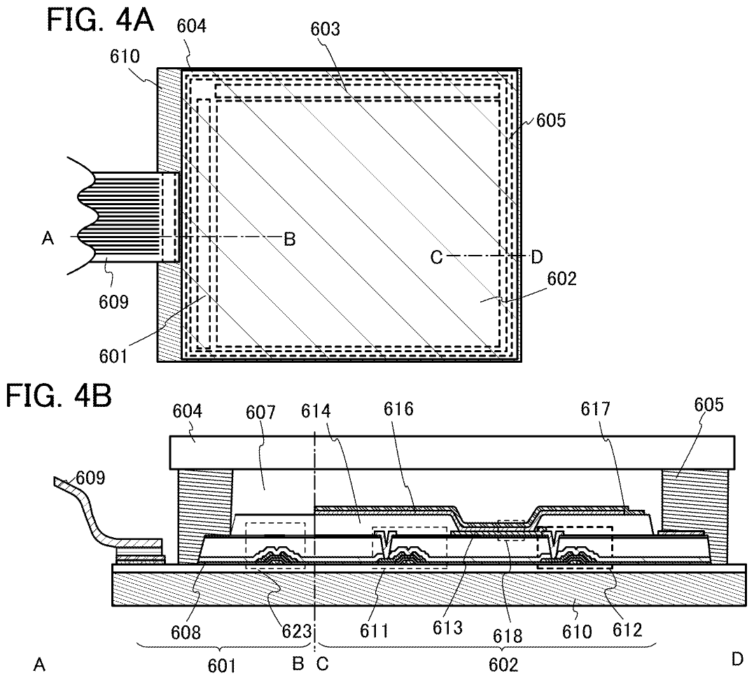

In this embodiment, a light-emitting apparatus fabricated using the light-emitting device described in any one of Embodiment 1 to Embodiment 6 is described with reference to FIG. 4. FIG. 4A is a top view illustrating the light-emitting apparatus, and FIG. 4B is a cross-sectional view taken along A-B and C-D in FIG. 4A. This light-emitting apparatus includes a driver circuit portion (source line driver circuit 601), a pixel portion 602, and a driver circuit portion (gate line driver circuit 603) that are to control light emission of light-emitting devices and are illustrated with dotted lines. Reference numeral 604 denotes a sealing substrate; 605, a sealant; and 607, a space surrounded by the sealant 605.

A lead wiring 608 is a wiring for transmitting signals to be input to the source line driver circuit 601 and the gate line driver circuit 603 and receives a video signal, a clock signal, a start signal, a reset signal, or the like from an FPC (flexible printed circuit) 609 serving as an external input terminal. Although only the FPC is illustrated here, a printed wiring board (PWB) may be attached to the FPC. The light-emitting apparatus in this specification includes, in its category, not only the light-emitting apparatus itself but also the light-emitting apparatus provided with the FPC or the PWB.

Next, a cross-sectional structure is described with reference to FIG. 4B. The driver circuit portions and the pixel portion are formed over an element substrate 610; here, the source line driver circuit 601, which is a driver circuit portion, and one pixel in the pixel portion 602 are illustrated.

The element substrate 610 may be formed using a substrate containing glass, quartz, an organic resin, a metal, an alloy, a semiconductor, or the like or a plastic substrate formed of FRP (Fiber Reinforced Plastics), PVF (polyvinyl fluoride), polyester, an acrylic resin, or the like.

There is no particular limitation on the structure of transistors used in pixels or driver circuits. For example, inverted staggered transistors may be used, or staggered transistors may be used. Furthermore, top-gate transistors or bottom-gate transistors may be used. A semiconductor material used for the transistors is not particularly limited, and for example, silicon, germanium, silicon carbide, gallium nitride, or the like can be used. Alternatively, an oxide semiconductor containing at least one of indium, gallium, and zinc, such as an In—Ga—Zn-based metal oxide, may be used.

There is no particular limitation on the crystallinity of a semiconductor material used for the transistors, and an amorphous semiconductor or a semiconductor having crystallinity (a microcrystalline semiconductor, a polycrystalline semiconductor, a single crystal semiconductor, or a semiconductor partly including crystal regions) may be used. A semiconductor having crystallinity is preferably used because deterioration of the transistor characteristics can be inhibited.

Here, an oxide semiconductor is preferably used for semiconductor devices such as the transistors provided in the pixels or the driver circuits and transistors used for after-mentioned touch sensors and the like. In particular, an oxide semiconductor having a wider band gap than silicon is preferably used. When an oxide semiconductor having a wider band gap than silicon is used, the off-state current of the transistors can be reduced.

The oxide semiconductor preferably contains at least indium (In) or zinc (Zn). Further preferably, the oxide semiconductor contains an oxide represented by an In-M-Zn-based oxide (M represents a metal such as Al, Ti, Ga, Ge, Y, Zr, Sn, La, Ce, or Hf).

As a semiconductor layer, it is particularly preferable to use an oxide semiconductor film including a plurality of crystal parts whose c-axes are aligned perpendicular to a surface on which the semiconductor layer is formed or the top surface of the semiconductor layer and in which the adjacent crystal parts have no grain boundary.

The use of such materials for the semiconductor layer makes it possible to provide a highly reliable transistor in which a change in the electrical characteristics is inhibited.

Charge accumulated in a capacitor through a transistor including the above-described semiconductor layer can be held for a long time because of the low off-state current of the transistor. When such a transistor is used in a pixel, operation of a driver circuit can be stopped while a gray scale of an image displayed in each display region is maintained. As a result, an electronic device with extremely low power consumption can be obtained.

For stable characteristics or the like of the transistor, a base film is preferably provided. The base film can be formed with a single layer or stacked layers using an inorganic insulating film such as a silicon oxide film, a silicon nitride film, a silicon oxynitride film, or a silicon nitride oxide film. The base film can be formed by a sputtering method, a CVD (Chemical Vapor Deposition) method (e.g., a plasma CVD method, a thermal CVD method, or an MOCVD (Metal Organic CVD) method), an ALD (Atomic Layer Deposition) method, a coating method, a printing method, or the like. Note that the base film does not have to be provided if not necessary.

Note that an FET 623 is illustrated as a transistor formed in the source line driver circuit 601. The driver circuit is formed with any of a variety of circuits such as a CMOS circuit, a PMOS circuit, or an NMOS circuit. Although a driver-integrated type in which the driver circuit is formed over the substrate is illustrated in this embodiment, the driver circuit is not necessarily formed over the substrate, and the driver circuit can be formed outside, not over the substrate.

The pixel portion 602 is formed with a plurality of pixels including a switching FET 611, a current control FET 612, and a first electrode 613 electrically connected to a drain of the current control FET 612; however, without being limited thereto, a pixel portion in which three or more FETs and a capacitor are combined may be employed.

Note that an insulator 614 is formed to cover an end portion of the first electrode 613. Here, the insulator 614 can be formed using a positive photosensitive acrylic resin film.

In order to improve the coverage with an EL layer or the like which is formed later, the insulator 614 is formed to have a curved surface with curvature at its upper or lower end portion. For example, in the case where a positive photosensitive acrylic resin is used as a material for the insulator 614, only the upper end portion of the insulator 614 preferably has a curved surface with a curvature radius (greater than or equal to 0.2 μm and less than or equal to 3 μm). As the insulator 614, either a negative photosensitive resin or a positive photosensitive resin can be used.

An EL layer 616 and a second electrode 617 are formed over the first electrode 613. Here, as a material used for the first electrode 613 functioning as an anode, a material with a high work function is desirably used. For example, a single-layer film of an ITO film, an indium tin oxide film containing silicon, an indium oxide film containing zinc oxide at 2 wt % or higher and 20 wt % or lower, a titanium nitride film, a chromium film, a tungsten film, a Zn film, a Pt film, or the like, a stacked layer of a titanium nitride film and a film containing aluminum as its main component, a three-layer structure of a titanium nitride film, a film containing aluminum as its main component, and a titanium nitride film, or the like can be used. The stacked-layer structure enables low wiring resistance, favorable ohmic contact, and a function as an anode.

The EL layer 616 is formed by any of a variety of methods such as an evaporation method using an evaporation mask, an inkjet method, and a spin coating method. The EL layer 616 has the structure described in any one of Embodiment 1 to Embodiment 6. As another material included in the EL layer 616, a low molecular compound or a high molecular compound (including an oligomer or a dendrimer) may be used.

As a material used for the second electrode 617, which is formed over the EL layer 616 and functions as a cathode, a material with a low work function (e.g., Al, Mg, Li, Ca, or an alloy or a compound thereof (e.g., MgAg, Mgln, or AlLi)) is preferably used. Note that in the case where light generated in the EL layer 616 passes through the second electrode 617, it is preferable to use, for the second electrode 617, a stacked layer of a thin metal film and a transparent conductive film (e.g., ITO, indium oxide containing zinc oxide at 2 wt % or higher and 20 wt % or lower, indium tin oxide containing silicon, or zinc oxide (ZnO)).

Note that a light-emitting device 618 is formed with the first electrode 613, the EL layer 616, and the second electrode 617. The light-emitting device is the light-emitting device described in any one of Embodiment 1 to Embodiment 6. A plurality of light-emitting devices are formed in the pixel portion, and the light-emitting apparatus of this embodiment may include both the light-emitting device described in any one of Embodiment 1 to Embodiment 6 and a light-emitting device having a different structure.

The sealing substrate 604 is attached to the element substrate 610 with the sealant 605, so that the light-emitting device 618 is provided in the space 607 surrounded by the element substrate 610, the sealing substrate 604, and the sealant 605. The space 607 is filled with a filler; it is filled with an inert gas (e.g., nitrogen or argon) in some cases, and filled with the sealant in some cases. It is preferable that the sealing substrate have a recessed portion provided with a desiccant, in which case degradation due to the influence of moisture can be inhibited.

Note that an epoxy resin or glass frit is preferably used for the sealant 605. It is preferable that such a material transmit moisture or oxygen as little as possible. As the material used for the sealing substrate 604, in addition to a glass substrate and a quartz substrate, a plastic substrate formed of FRP (Fiber Reinforced Plastics), PVF (polyvinyl fluoride), polyester, an acrylic resin, or the like can be used.

Although not illustrated in FIG. 4, a protective film may be provided over the second electrode. The protective film is formed using an organic resin film or an inorganic insulating film. The protective film may be formed so as to cover an exposed portion of the sealant 605. The protective film can be provided to cover surfaces and side surfaces of the pair of substrates and exposed side surfaces of a sealing layer, an insulating layer, and the like.

The protective film can be formed using a material that does not easily transmit an impurity such as water. Thus, diffusion of an impurity such as water from the outside into the inside can be effectively inhibited.

As a material of the protective film, an oxide, a nitride, a fluoride, a sulfide, a ternary compound, a metal, a polymer, or the like can be used. For example, it is possible to use a material containing aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, indium oxide, or the like; a material containing aluminum nitride, hafnium nitride, silicon nitride, tantalum nitride, titanium nitride, niobium nitride, molybdenum nitride, zirconium nitride, gallium nitride, or the like; a material containing a nitride containing titanium and aluminum, an oxide containing titanium and aluminum, an oxide containing aluminum and zinc, a sulfide containing manganese and zinc, a sulfide containing cerium and strontium, an oxide containing erbium and aluminum, an oxide containing yttrium and zirconium, or the like.

The protective film is preferably formed using a deposition method with favorable step coverage. One such method is an atomic layer deposition (ALD) method. A material that can be deposited by an ALD method is preferably used for the protective film. With the use of an ALD method, a dense protective film with reduced defects such as cracks or pinholes or with a uniform thickness can be formed. Furthermore, damage caused to a process member in forming the protective film can be reduced.

By an ALD method, for example, a uniform protective film with few defects can be formed even on a surface with a complex uneven shape or upper, side, and lower surfaces of a touch panel.

As described above, the light-emitting apparatus fabricated using the light-emitting device described in any one of Embodiment 1 to Embodiment 6 can be obtained.

The light-emitting apparatus in this embodiment is fabricated using the light-emitting device described in any one of Embodiment 1 to Embodiment 6 and thus can have favorable characteristics. Specifically, since the light-emitting device described in any one of Embodiment 1 to Embodiment 6 has favorable emission efficiency, the light-emitting apparatus with low power consumption can be obtained.

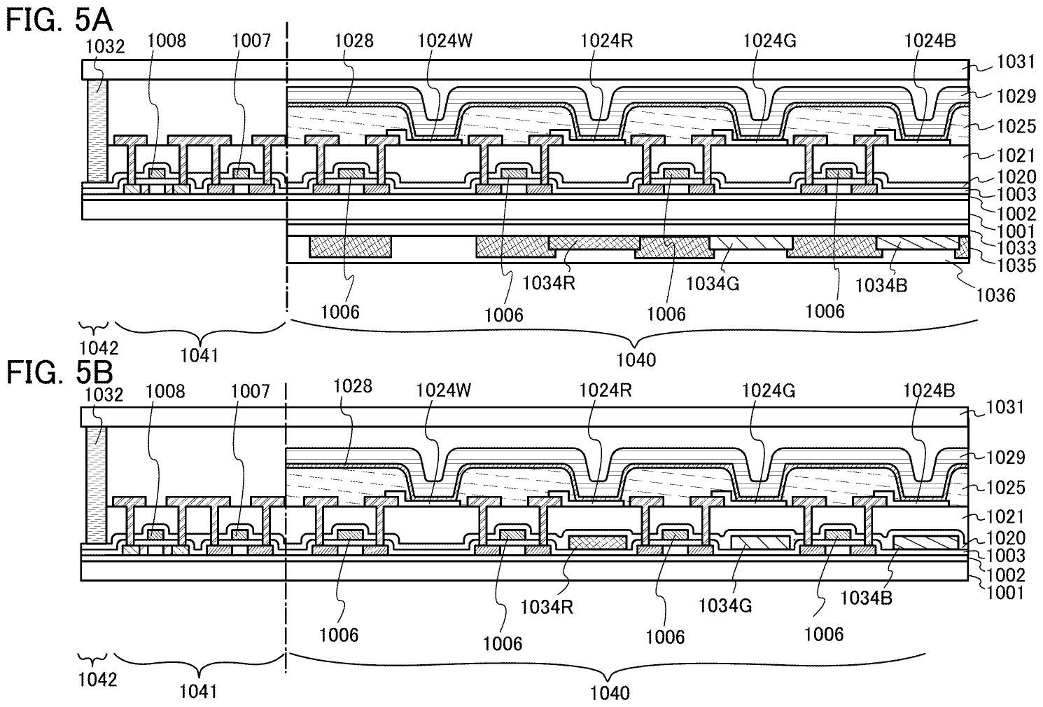

FIG. 5 illustrates examples of a light-emitting apparatus in which full color display is achieved by formation of light-emitting devices exhibiting white light emission and provision of coloring layers (color filters) and the like. FIG. 5A illustrates a substrate 1001, a base insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, first electrodes 1024W, 1024R, 1024G, and 1024B of the light-emitting devices, a partition 1025, an EL layer 1028, a second electrode 1029 of the light-emitting devices, a sealing substrate 1031, a sealant 1032, and the like.

In FIG. 5A, coloring layers (a red coloring layer 1034R, a green coloring layer 1034G, and a blue coloring layer 1034B) are provided on a transparent base material 1033. A black matrix 1035 may be additionally provided. The transparent base material 1033 provided with the coloring layers and the black matrix is aligned and fixed to the substrate 1001. Note that the coloring layers and the black matrix 1035 are covered with an overcoat layer 1036. In FIG. 5A, a light-emitting layer from which light is emitted to the outside without passing through the coloring layer and light-emitting layers from which light is emitted to the outside, passing through the coloring layers of the respective colors are shown. Since light that does not pass through the coloring layer is white and light that passes through the coloring layer is red, green, or blue, an image can be expressed by pixels of the four colors.

FIG. 5B shows an example in which the coloring layers (the red coloring layer 1034R, the green coloring layer 1034G, and the blue coloring layer 1034B) are formed between the gate insulating film 1003 and the first interlayer insulating film 1020. As in the structure, the coloring layers may be provided between the substrate 1001 and the sealing substrate 1031.

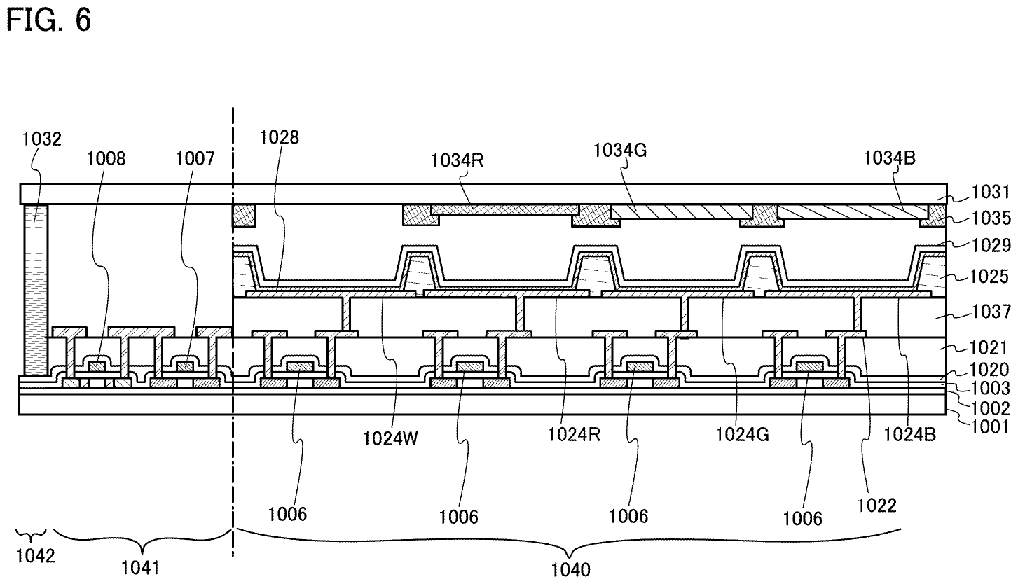

The above-described light-emitting apparatus has a structure in which light is extracted from the substrate 1001 side where FETs are formed (a bottom emission structure), but may have a structure in which light is extracted from the sealing substrate 1031 side (a top emission structure). FIG. 6 shows a cross-sectional view of a top-emission light-emitting apparatus. In this case, a substrate that does not transmit light can be used as the substrate 1001. The top-emission light-emitting apparatus is formed in a manner similar to that of the bottom-emission light-emitting apparatus until a connection electrode that connects the FET and the anode of the light-emitting device is formed. Then, a third interlayer insulating film 1037 is formed to cover an electrode 1022. This insulating film may have a planarization function. The third interlayer insulating film 1037 can be formed using a material similar to that of the second interlayer insulating film, and can alternatively be formed using any of other known materials.

The first electrodes 1024W, 1024R, 1024G, and 1024B of the light-emitting devices are each an anode here, but may each be a cathode. In the case of the top-emission light-emitting apparatus such as one in FIG. 6, the first electrodes are preferably reflective electrodes. The structure of the EL layer 1028 is such a structure as that of the unit 103 described in any one of Embodiment 1 to Embodiment 6, and an element structure with which white light emission can be obtained.

In the case of such a top-emission structure as in FIG. 6, sealing can be performed with the sealing substrate 1031 on which the coloring layers (the red coloring layer 1034R, the green coloring layer 1034G, and the blue coloring layer 1034B) are provided. The sealing substrate 1031 may be provided with the black matrix 1035 that is positioned between pixels. The coloring layers (the red coloring layer 1034R, the green coloring layer 1034G, and the blue coloring layer 1034B) or the black matrix may be covered with the overcoat layer 1036. Note that a substrate having a light-transmitting property is used as the sealing substrate 1031. Although an example in which full color display is performed using four colors of red, green, blue, and white is shown here, there is no particular limitation and full color display may be performed using four colors of red, yellow, green, and blue or three colors of red, green, and blue.

In the top-emission light-emitting apparatus, a microcavity structure can be favorably employed. A light-emitting device with a microcavity structure can be obtained with the use of a reflective electrode as the first electrode and a transflective electrode as the second electrode. The light-emitting device with a microcavity structure includes at least an EL layer between the reflective electrode and the transflective electrode, and the EL layer includes at least a light-emitting layer serving as a light-emitting region.

Note that the reflective electrode is a film having a visible light reflectivity of 40% to 100%, preferably 70% to 100%, and a resistivity of 1×10⁻² Ωcm or lower. In addition, the transflective electrode is a film having a visible light reflectivity of 20% to 80%, preferably 40% to 70%, and a resistivity of 1×10⁻² Ωcm or lower.

Light emitted from the light-emitting layer included in the EL layer is reflected and resonated by the reflective electrode and the transflective electrode.

In the light-emitting device, by changing thicknesses of the transparent conductive film, the above-described composite material, the carrier-transport material, or the like, the optical path length between the reflective electrode and the transflective electrode can be changed. Thus, light with a wavelength that is resonated between the reflective electrode and the transflective electrode can be intensified while light with a wavelength that is not resonated therebetween can be attenuated.

Note that light that is reflected back by the reflective electrode (first reflected light) considerably interferes with light that directly enters the transflective electrode from the light-emitting layer (first incident light). For this reason, the optical path length between the reflective electrode and the light-emitting layer is preferably adjusted to (2n−1)λ/4 (n is a natural number of 1 or larger and λ is a wavelength of light to be amplified). By adjusting the optical path length, the phases of the first reflected light and the first incident light can be aligned with each other and the light emitted from the light-emitting layer can be further amplified.

Note that in the above structure, the EL layer may include a plurality of light-emitting layers or may include a single light-emitting layer; for example, in combination with the structure of the above-described tandem light-emitting device, a plurality of EL layers each including a single or a plurality of light-emitting layer(s) may be provided in one light-emitting device with a charge-generation layer positioned between the EL layers.

With the microcavity structure, emission intensity with a specific wavelength in the front direction can be increased, whereby power consumption can be reduced. Note that in the case of a light-emitting apparatus that displays images with subpixels of four colors, red, yellow, green, and blue, the light-emitting apparatus can have favorable characteristics because the luminance can be increased owing to yellow light emission and each subpixel can employ a microcavity structure suitable for wavelengths of the corresponding color.

The light-emitting apparatus in this embodiment is fabricated using the light-emitting device described in any one of Embodiment 1 to Embodiment 6 and thus can have favorable characteristics. Specifically, since the light-emitting device described in any one of Embodiment 1 to Embodiment 6 has favorable emission efficiency, the light-emitting apparatus with low power consumption can be obtained.

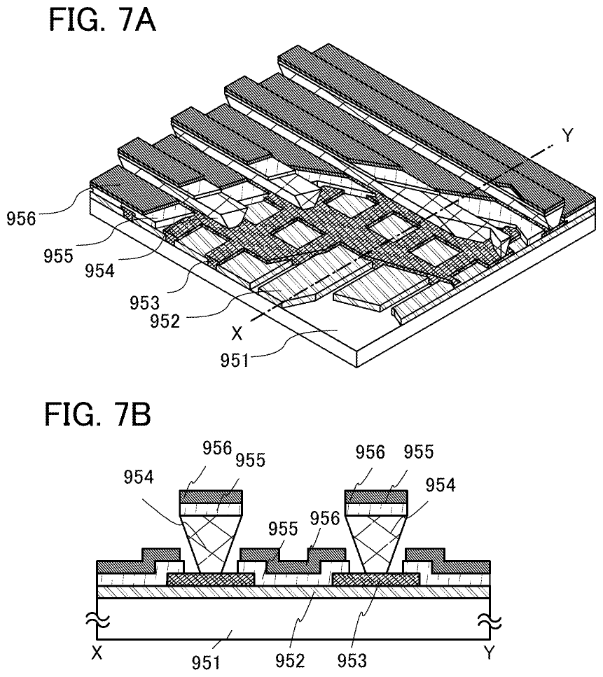

The active matrix light-emitting apparatus is described above, whereas a passive matrix light-emitting apparatus is described below. FIG. 7 illustrates a passive matrix light-emitting apparatus fabricated using the present invention. Note that FIG. 7A is a perspective view illustrating the light-emitting apparatus, and FIG. 7B is a cross-sectional view taken along X-Y in FIG. 7A. In FIG. 7, over a substrate 951, an EL layer 955 is provided between an electrode 952 and an electrode 956. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. In other words, a cross section taken along the direction of the short side of the partition layer 954 is trapezoidal, and the lower side (a side that is parallel to the surface of the insulating layer 953 and is in contact with the insulating layer 953) is shorter than the upper side (a side that is parallel to the surface of the insulating layer 953 and is not in contact with the insulating layer 953). The partition layer 954 thus provided can prevent defects in the light-emitting device due to static electricity or the like. The passive matrix light-emitting apparatus also uses the light-emitting device described in any one of Embodiment 1 to Embodiment 6; thus, the light-emitting apparatus can have favorable reliability or low power consumption.

In the light-emitting apparatus described above, many minute light-emitting devices arranged in a matrix can each be controlled; thus, the light-emitting apparatus can be suitably used as a display apparatus for displaying images.

This embodiment can be freely combined with any of the other embodiments.

### Embodiment 9

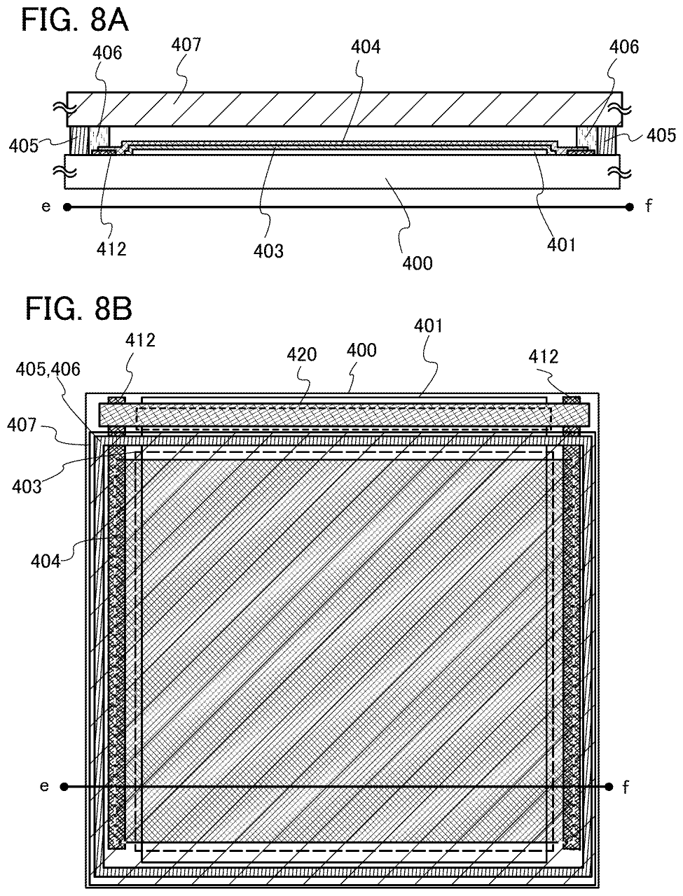

In this embodiment, an example in which the light-emitting device described in any one of Embodiment 1 to Embodiment 6 is used for a lighting device is described with reference to FIG. 8. FIG. 8B is a top view of the lighting device, and FIG. 8A is a cross-sectional view taken along e-f in FIG. 8B.

In the lighting device in this embodiment, a first electrode 401 is formed over a substrate 400 that is a support and has a light-transmitting property. The first electrode 401 corresponds to the first electrode 101 in any one of Embodiment 1 to Embodiment 6. In the case where light emission is extracted from the first electrode 401 side, the first electrode 401 is formed with a material having a light-transmitting property.

A pad 412 for supplying a voltage to a second electrode 404 is formed over the substrate 400.

An EL layer 403 is formed over the first electrode 401. The EL layer 403 has a structure corresponding to the structure of the unit 103 in any one of Embodiment 1 to Embodiment 6, the structure in which the unit 103(12) and the intermediate layer 106 are combined, or the like. Refer to the corresponding description for these structures.

The second electrode 404 is formed to cover the EL layer 403. The second electrode 404 corresponds to the second electrode 102 in any one of Embodiment 1 to Embodiment 6. In the case where light emission is extracted from the first electrode 401 side, the second electrode 404 is formed with a material having high reflectivity. The second electrode 404 is supplied with a voltage when connected to the pad 412.

As described above, the lighting device described in this embodiment includes a light-emitting device including the first electrode 401, the EL layer 403, and the second electrode 404. Since the light-emitting device is a light-emitting device with high emission efficiency, the lighting device in this embodiment can have low power consumption.

The substrate 400 provided with the light-emitting device having the above structure is fixed to a sealing substrate 407 with sealants 405 and 406 and sealing is performed, whereby the lighting device is completed. It is possible to use only either the sealant 405 or the sealant 406. In addition, the inner sealant 406 (not shown in FIG. 8B) can be mixed with a desiccant, which enables moisture to be adsorbed, resulting in improved reliability.

When parts of the pad 412 and the first electrode 401 are provided to extend to the outside of the sealants 405 and 406, those can serve as external input terminals. An IC chip 420 mounted with a converter or the like may be provided over the external input terminals.

The lighting device described in this embodiment uses the light-emitting device described in any one of Embodiment 1 to Embodiment 6 as an EL element; thus, the lighting device can have low power consumption.

### Embodiment 10

In this embodiment, examples of electronic devices each partly including the light-emitting device described in any one of Embodiment 1 to Embodiment 6 are described. The light-emitting device described in any one of Embodiment 1 to Embodiment 6 is a light-emitting device with favorable emission efficiency and low power consumption. As a result, the electronic devices described in this embodiment can be electronic devices each including a light-emitting portion with low power consumption.

Examples of the electronic device including the above light-emitting device include television devices (also referred to as TV or television receivers), monitors for computers and the like, digital cameras, digital video cameras, digital photo frames, cellular phones (also referred to as mobile phones or mobile phone devices), portable game machines, portable information terminals, audio playback devices, and large game machines such as pachinko machines. Specific examples of these electronic devices are shown below.

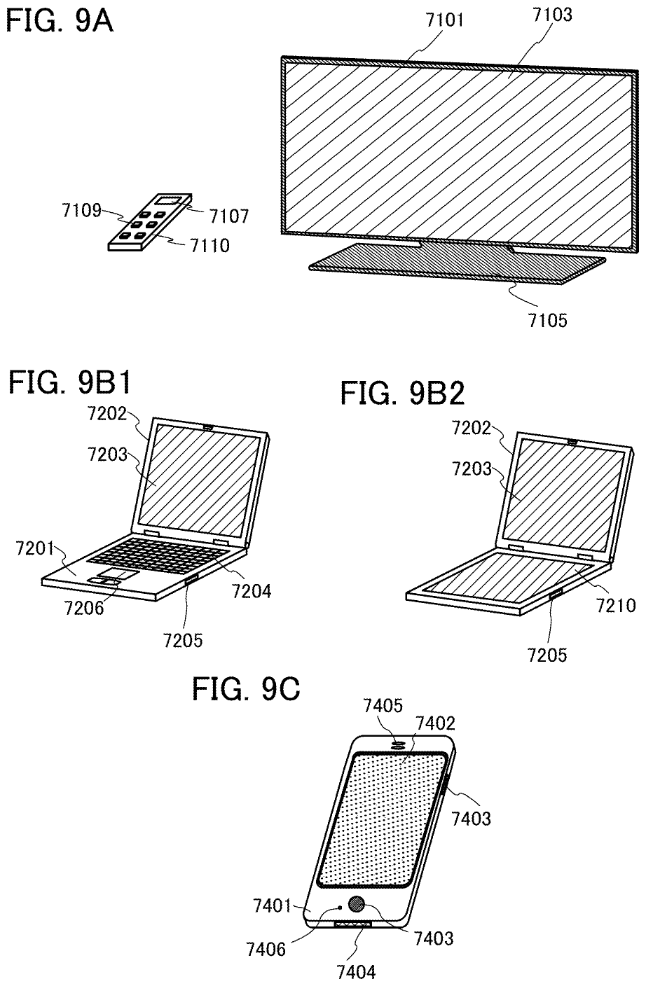

FIG. 9A illustrates an example of a television device. In the television device, a display portion 7103 is incorporated in a housing 7101. Here, the housing 7101 is supported by a stand 7105. Images can be displayed on the display portion 7103, and the light-emitting devices described in any one of Embodiment 1 to Embodiment 6 are arranged in a matrix in the display portion 7103.

The television device can be operated with an operation switch of the housing 7101 or a separate remote controller 7110. With operation keys 7109 of the remote controller 7110, channels and volume can be operated and images displayed on the display portion 7103 can be operated. Furthermore, the remote controller 7110 may be provided with a display portion 7107 for displaying data output from the remote controller 7110.

Note that the television device has a structure including a receiver, a modem, or the like. With the use of the receiver, a general television broadcast can be received, and moreover, when the television device is connected to a communication network with or without wires via the modem, one-way (from a sender to a receiver) or two-way (between a sender and a receiver or between receivers) data communication can be performed.

FIG. 9B1 shows a computer that includes a main body 7201, a housing 7202, a display portion 7203, a keyboard 7204, an external connection port 7205, a pointing device 7206, and the like. Note that this computer is fabricated using the light-emitting devices described in any one of Embodiment 1 to Embodiment 6 arranged in a matrix in the display portion 7203. The computer in FIG. 9B1 may be such a mode as illustrated in FIG. 9B2. The computer in FIG. 9B2 is provided with a second display portion 7210 instead of the keyboard 7204 and the pointing device 7206. The second display portion 7210 is of a touch-panel type, and input can be performed by operating display for input displayed on the second display portion 7210 with a finger or a dedicated pen. The second display portion 7210 can also display images other than the display for input. The display portion 7203 may also be a touch panel. Connecting the two screens with a hinge can prevent troubles such as a crack in or damage to the screens caused when the computer is stored or carried.

FIG. 9C illustrates an example of a portable terminal. The portable terminal includes operation buttons 7403, an external connection port 7404, a speaker 7405, a microphone 7406, and the like in addition to a display portion 7402 incorporated in a housing 7401. Note that the portable terminal includes the display portion 7402 that is fabricated by arranging the light-emitting devices described in any one of Embodiment 1 to Embodiment 6 in a matrix.

The portable terminal illustrated in FIG. 9C can have a structure in which information can be input by touching the display portion 7402 with a finger or the like. In this case, operations such as making a call and creating an e-mail can be performed by touching the display portion 7402 with a finger or the like.

The display portion 7402 has mainly three screen modes. The first mode is a display mode mainly for displaying images. The second mode is an input mode mainly for inputting data such as text. The third mode is a display-and-input mode in which the two modes, the display mode and the input mode, are combined.

For example, in the case of making a call or creating an e-mail, the text input mode mainly for inputting text is selected for the display portion 7402 so that text displayed on the screen can be input. In this case, it is preferable to display a keyboard or number buttons on almost the entire screen of the display portion 7402.

When a sensing device including a sensor such as a gyroscope sensor or an acceleration sensor for detecting inclination is provided inside the portable terminal, display on the screen of the display portion 7402 can be automatically changed by determining the orientation of the portable terminal (whether the portable terminal is placed vertically or horizontally).

The screen modes are switched by touching the display portion 7402 or operating the operation buttons 7403 of the housing 7401. Alternatively, the screen modes can be switched depending on the kind of images displayed on the display portion 7402. For example, when a signal of an image displayed on the display portion is moving image data, the screen mode is switched to the display mode. When the signal is text data, the screen mode is switched to the input mode.

Moreover, in the input mode, when input by touching the display portion 7402 is not performed for a certain period while a signal sensed by an optical sensor in the display portion 7402 is sensed, the screen mode may be controlled so as to be switched from the input mode to the display mode.

The display portion 7402 can also function as an image sensor. For example, an image of a palm print, a fingerprint, or the like is taken when the display portion 7402 is touched with the palm or the finger, whereby personal authentication can be performed. Furthermore, by providing a backlight that emits near-infrared light or a sensing light source that emits near-infrared light in the display portion, an image of a finger vein, a palm vein, or the like can be taken.

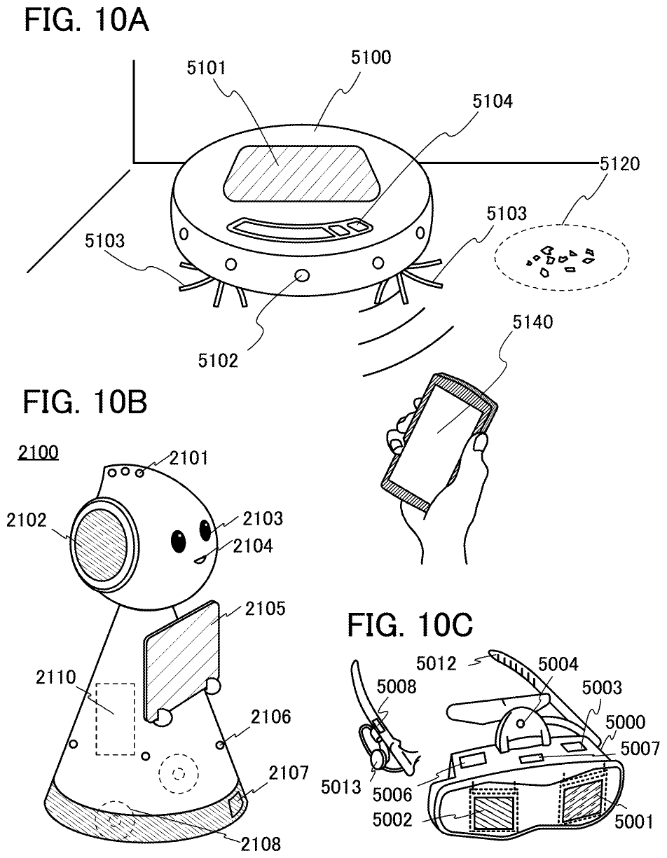

FIG. 10A is a schematic view showing an example of a cleaning robot.

A cleaning robot 5100 includes a display 5101 on its top surface, a plurality of cameras 5102 on its side surface, a brush 5103, and operation buttons 5104. Although not illustrated, the bottom surface of the cleaning robot 5100 is provided with a tire, an inlet, and the like. Furthermore, the cleaning robot 5100 includes various sensors such as an infrared sensor, an ultrasonic sensor, an acceleration sensor, a piezoelectric sensor, an optical sensor, and a gyroscope sensor. The cleaning robot 5100 has a wireless communication means.

The cleaning robot 5100 is self-propelled, detects dust 5120, and vacuums the dust through the inlet provided on the bottom surface.

The cleaning robot 5100 can determine whether there is an obstacle such as a wall, furniture, or a step by analyzing images taken by the cameras 5102. When an object that is likely to be caught in the brush 5103, such as a wire, is detected by image analysis, the rotation of the brush 5103 can be stopped.

The display 5101 can display the remaining capacity of a battery, the amount of vacuumed dust, or the like. The display 5101 may display a path on which the cleaning robot 5100 has run. The display 5101 may be a touch panel, and the operation buttons 5104 may be provided on the display 5101.

The cleaning robot 5100 can communicate with a portable electronic device 5140 such as a smartphone. Images taken by the cameras 5102 can be displayed on the portable electronic device 5140. Accordingly, an owner of the cleaning robot 5100 can monitor his/her room even when the owner is not at home. The owner can also check the display on the display 5101 by the portable electronic device such as a smartphone.

The light-emitting apparatus of one embodiment of the present invention can be used for the display 5101.

A robot 2100 illustrated in FIG. 10B includes an arithmetic device 2110, an illuminance sensor 2101, a microphone 2102, an upper camera 2103, a speaker 2104, a display 2105, a lower camera 2106, an obstacle sensor 2107, and a moving mechanism 2108.

The microphone 2102 has a function of detecting a speaking voice of a user, an environmental sound, and the like. The speaker 2104 has a function of outputting sound. The robot 2100 can communicate with a user by using the microphone 2102 and the speaker 2104.

The display 2105 has a function of displaying various kinds of information. The robot 2100 can display information desired by a user on the display 2105. The display 2105 may be provided with a touch panel. Moreover, the display 2105 may be a detachable information terminal, in which case charging and data communication can be performed when the display 2105 is set at the home position of the robot 2100.

The upper camera 2103 and the lower camera 2106 each have a function of taking an image of the surroundings of the robot 2100. The obstacle sensor 2107 can detect an obstacle in the direction where the robot 2100 advances with the moving mechanism 2108. The robot 2100 can move safely by recognizing the surroundings with the upper camera 2103, the lower camera 2106, and the obstacle sensor 2107. The light-emitting apparatus of one embodiment of the present invention can be used for the display 2105.

FIG. 10C is a diagram illustrating an example of a goggle-type display. The goggle-type display includes, for example, a housing 5000, a display portion 5001, a speaker 5003, an LED lamp 5004, operation keys (including a power switch or an operation switch), a connection terminal 5006, a sensor 5007 (a sensor having a function of measuring force, displacement, position, speed, acceleration, angular velocity, rotational frequency, distance, light, liquid, magnetism, temperature, a chemical substance, sound, time, hardness, an electric field, current, voltage, power, radiation, flow rate, humidity, gradient, oscillation, odor, or infrared ray), a microphone 5008, a display portion 5002, a support 5012, and an earphone 5013.

The light-emitting apparatus of one embodiment of the present invention can be used for the display portion 5001 and the display portion 5002.

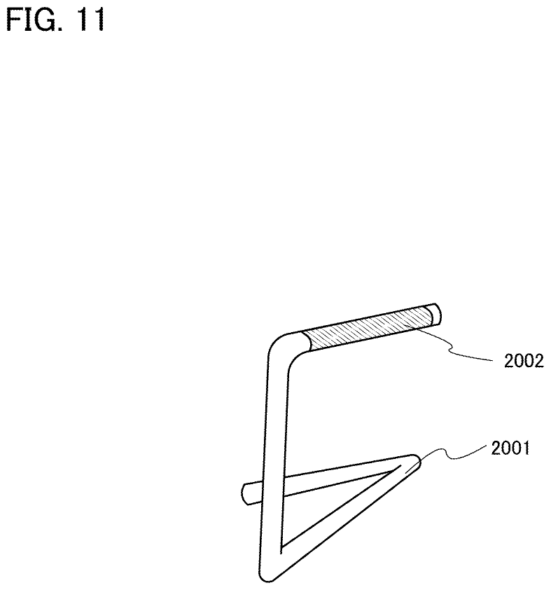

FIG. 11 shows an example where the light-emitting device described in any one of Embodiment 1 to Embodiment 6 is used for a table lamp which is a lighting device. The table lamp illustrated in FIG. 11 includes a housing 2001 and a light source 2002, and the lighting device described in Embodiment 9 may be used for the light source 2002.

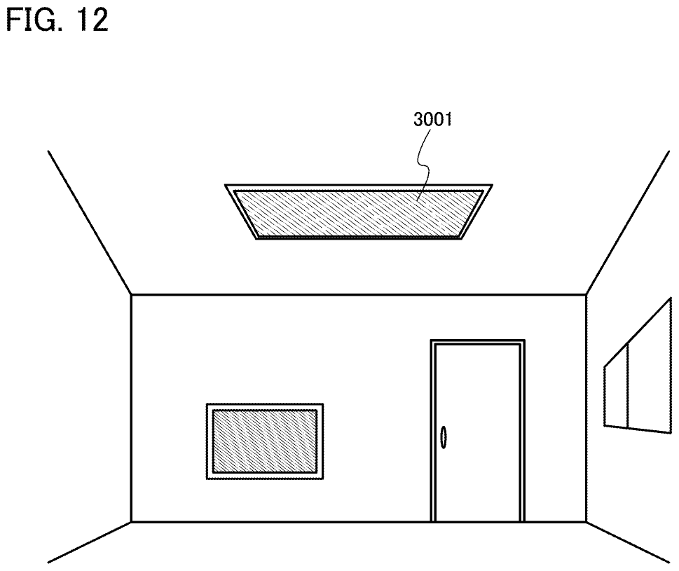

FIG. 12 shows an example where the light-emitting device described in any one of Embodiment 1 to Embodiment 6 is used for an indoor lighting device 3001. Since the light-emitting device described in any one of Embodiment 1 to Embodiment 6 is a light-emitting device with high emission efficiency, the lighting device can have low power consumption. In addition, the light-emitting device described in any one of Embodiment 1 to Embodiment 6 can have a larger area, and thus can be used for a large-area lighting device. Furthermore, the light-emitting device described in any one of Embodiment 1 to Embodiment 6 is thin, and thus can be used for a lighting device having a reduced thickness.

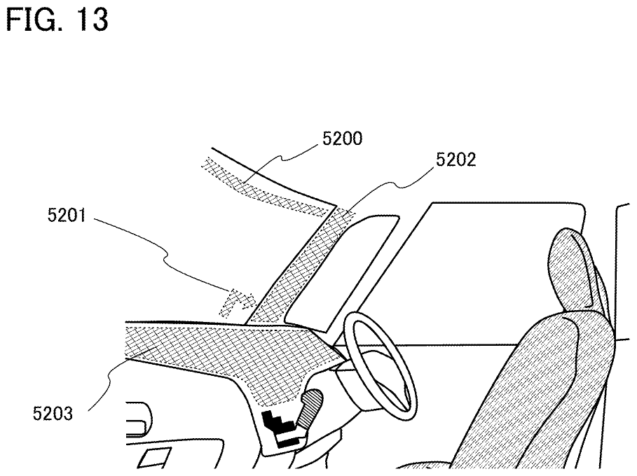

The light-emitting device described in any one of Embodiment 1 to Embodiment 6 can also be incorporated in a windshield or a dashboard of an automobile. FIG. 13 illustrates one mode in which the light-emitting device described in any one of Embodiment 1 to Embodiment 6 is used for a windshield or a dashboard of an automobile. A display region 5200 to a display region 5203 are each a display region provided using the light-emitting device described in any one of Embodiment 1 to Embodiment 6.

The display region 5200 and the display region 5201 are display apparatuses provided in the automobile windshield, in which the light-emitting devices described in any one of Embodiment 1 to Embodiment 6 are incorporated. When the light-emitting devices described in any one of Embodiment 1 to Embodiment 6 are fabricated using electrodes having light-transmitting properties as a first electrode and a second electrode, what is called see-through display apparatuses, through which the opposite side can be seen, can be obtained. Such see-through display apparatuses can be provided even in the automobile windshield without hindering the view. Note that in the case where a driving transistor or the like is provided, a transistor having a light-transmitting property, such as an organic transistor using an organic semiconductor material or a transistor using an oxide semiconductor, is preferably used.

The display region 5202 is a display apparatus provided in a pillar portion, in which the light-emitting devices described in any one of Embodiment 1 to Embodiment 6 are incorporated. The display region 5202 can compensate for the view hindered by the pillar by displaying an image taken by an imaging unit provided in the car body. Similarly, the display region 5203 provided in the dashboard portion can compensate for the view hindered by the car body by displaying an image taken by an imaging unit provided on the outside of the automobile; thus, blind areas can be eliminated to enhance the safety. Images that compensate for the areas that a driver cannot see enable the driver to ensure safety easily and comfortably.

The display region 5203 can provide a variety of kinds of information by displaying navigation data, a speedometer, a tachometer, a mileage, a fuel meter, a gearshift state, air-condition setting, and the like. The content or layout of the display can be changed freely in accordance with the preference of a user. Note that such information can also be displayed on the display region 5200 to the display region 5202. The display region 5200 to the display region 5203 can also be used as lighting devices.

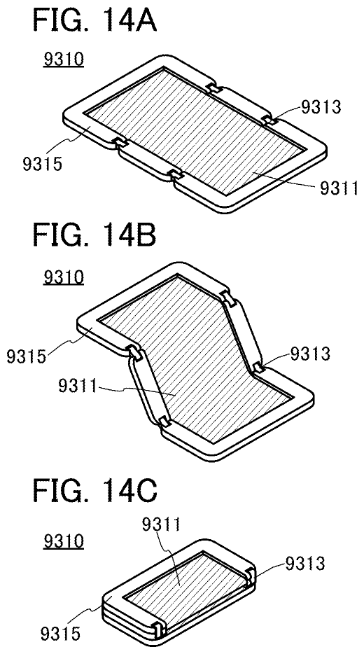

FIG. 14A to FIG. 14C illustrate a foldable portable information terminal 9310. FIG. 14A illustrates the portable information terminal 9310 that is opened. FIG. 14B illustrates the portable information terminal 9310 that is in the state of being changed from one of an opened state and a folded state to the other. FIG. 14C illustrates the portable information terminal 9310 that is folded. The portable information terminal 9310 is excellent in portability when folded, and is excellent in display browsability when opened because of a seamless large display region.

A functional panel 9311 is supported by three housings 9315 joined together by hinges 9313. Note that the functional panel 9311 may be a touch panel (an input/output device) including a touch sensor (an input device). By folding the functional panel 9311 at the hinges 9313 between two housings 9315, the portable information terminal 9310 can be reversibly changed in shape from the opened state to the folded state. The light-emitting apparatus of one embodiment of the present invention can be used for the functional panel 9311.

Note that the structures described in this embodiment can be combined with the structures described in any of Embodiment 1 to Embodiment 6 as appropriate.

As described above, the application range of the light-emitting apparatus including the light-emitting device described in any one of Embodiment 1 to Embodiment 6 is wide, so that this light-emitting apparatus can be applied to electronic devices in a variety of fields. With the use of the light-emitting device described in any one of Embodiment 1 to Embodiment 6, an electronic device with low power consumption can be obtained.

Note that this embodiment can be combined with any of the other embodiments in this specification as appropriate.

### Example 1

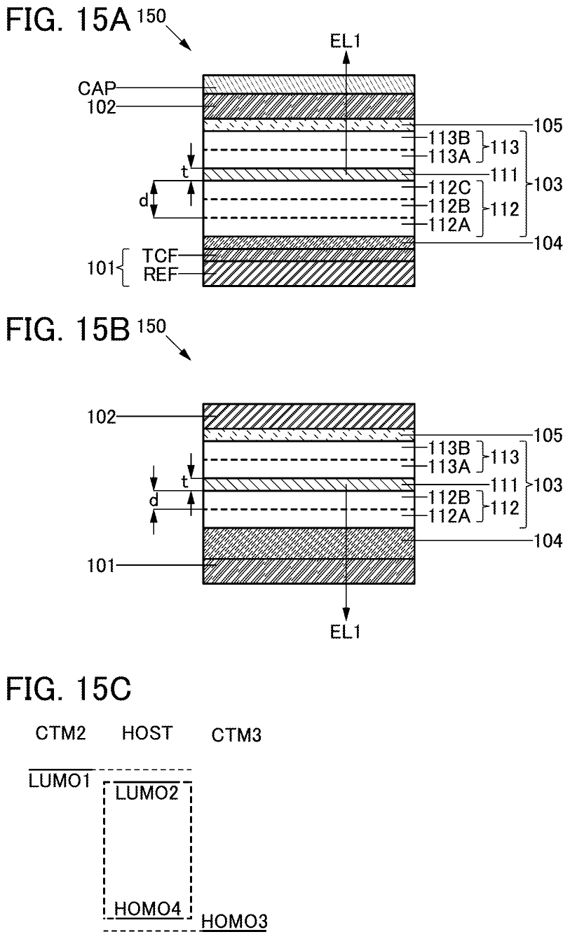

In this example, a fabricated light-emitting device 1 and a fabricated light-emitting device 2 of one embodiment of the present invention are described with reference to FIG. 15 to FIG. 27.

FIG. 15 is a diagram illustrating structures of the light-emitting devices of one embodiment of the present invention. FIG. 15A is a diagram illustrating the structure of the light-emitting device 1, FIG. 15B is a diagram illustrating the structure of the light-emitting device 2, and FIG. 15C is a diagram illustrating part of the structure of the light-emitting device.

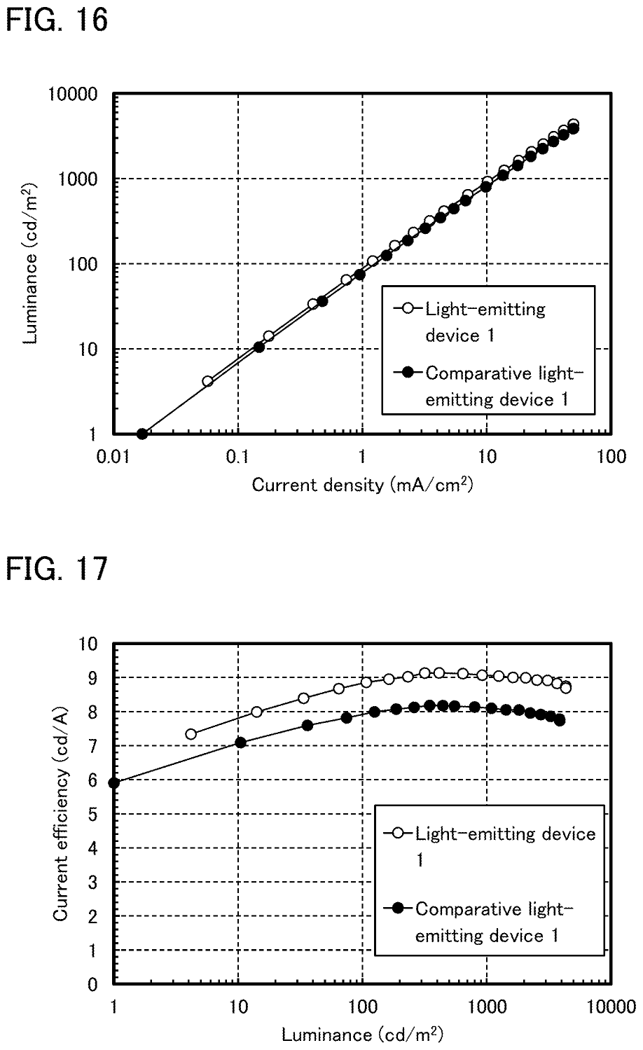

FIG. 16 is a graph showing current density—luminance characteristics of the light-emitting device 1 and a comparative light-emitting device 1.

FIG. 17 is a graph showing luminance—current efficiency characteristics of the light-emitting device 1 and the comparative light-emitting device 1.

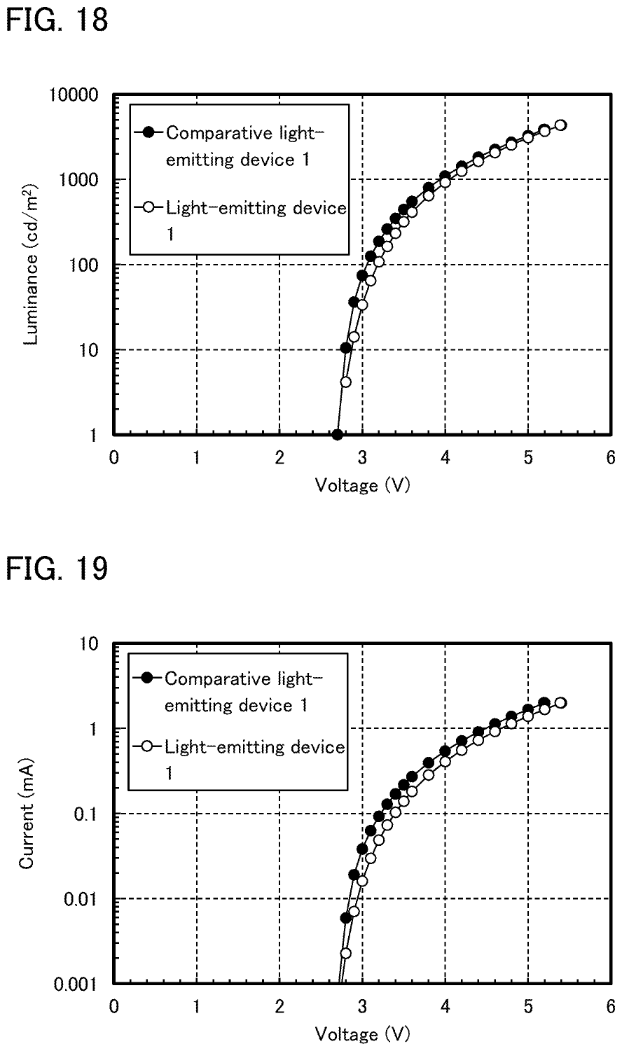

FIG. 18 is a graph showing voltage—luminance characteristics of the light-emitting device 1 and the comparative light-emitting device 1.

FIG. 19 is a graph showing voltage—current characteristics of the light-emitting device 1 and the comparative light-emitting device 1.

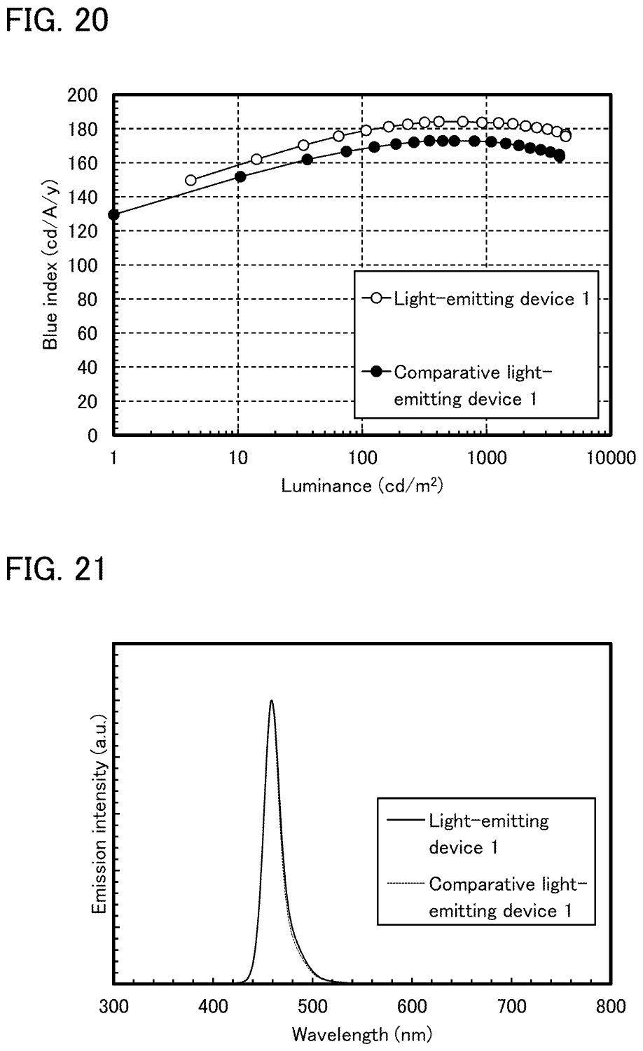

FIG. 20 is a graph showing luminance—blue index characteristics of the light-emitting device 1 and the comparative light-emitting device 1.

FIG. 21 is a graph showing emission spectra of the light-emitting device 1 and the comparative light-emitting device 1 each emitting light at a luminance of 1000 cd/m².

### <Light-Emitting Device 1>

The fabricated light-emitting device 1 described in this example includes a function of emitting the light EL1, the electrode 101, the electrode 102, and the unit 103 (see FIG. 15A).

The light EL1 has the spectrum ϕ1, and the spectrum ϕ1 has a maximum peak at the wavelength λ1 nm.

The electrode 102 includes a region overlapping with the electrode 101. The unit 103 includes a region positioned between the electrode 101 and the electrode 102, and the unit 103 includes the layer 111, the layer 112, and the layer 113.

The layer 111 includes a region positioned between the layer 112 and the layer 113, and the layer 111 contains a light-emitting material.

The layer 112 includes the layer 112A and the layer 112B. The layer 112B includes a region positioned between the layer 112A and the layer 111, and the layer 112B is in contact with the layer 112A.

The layer 112A has a refractive index of 2.02 with respect to light having a wavelength of 460 nm.

The layer 112B has a refractive index of 1.69 with respect to light having a wavelength of 460 nm, and the refractive index 1.69 is in the range of 1.4 and 1.75 and is lower than the refractive index 2.02.

There is a difference of 0.33 between the refractive index 1.69 and the refractive index 2.02.

In the fabricated light-emitting device 1 described in this example, the layer 111 has a thickness of 25 nm, and the layer 112A has a distance of 45 nm from the layer 111.

When the distance d is 45 nm, the thickness t is 25 nm, the wavelength λ is 460 nm, and the refractive index n2 is 1.69, the value of (d+t/2)×n2 is 97.125 nm. Furthermore, the value of 0.5×0.25×460 nm is 57.5 nm, and the value of 1.5×0.25×460 nm is 172.5 nm. That is, 97.125 nm is in the range of 57.5 nm and 172.5 nm.

### <<Structure of Light-Emitting Device 1>>

Table 1 shows the structure of the light-emitting device 1. Structural formulae of the materials used in the light-emitting devices described in this example are shown below.

TABLE 1

| | Reference | | Composition | Thickness/ |
|---|---|---|---|---|
| Component | numeral | Material | ratio | nm |
| Layer | CAP | DBT3P-II | | 70 |
| Electrode | 102 | Ag:Mg | 10:1 | 15 |
| Layer | 105 | LiF | | 1 |
| Layer | 113B | mPn-mDMePyPTzn:Liq | 1:1 | 20 |
| Layer | 113A | mFBPTzn | | 10 |
| Layer | 111 | Bnf(II)PhA:3,10PCA2Nbf(IV)-02 | 1:0.015 | 25 |
| Layer | 112C | PCBDBtBB-02 | | 10 |
| Layer | 112B | CTM2 | | 35 |
| Layer | 112A | PCBDBtBB-02 | | 70 |
| Layer | 104 | PCBDBtBB-02:OCHD-001 | 1:0.1 | 10 |
| Conductive film | TCF | ITSO | | 10 |
| Reflective film | REF | Ag | | 100 |

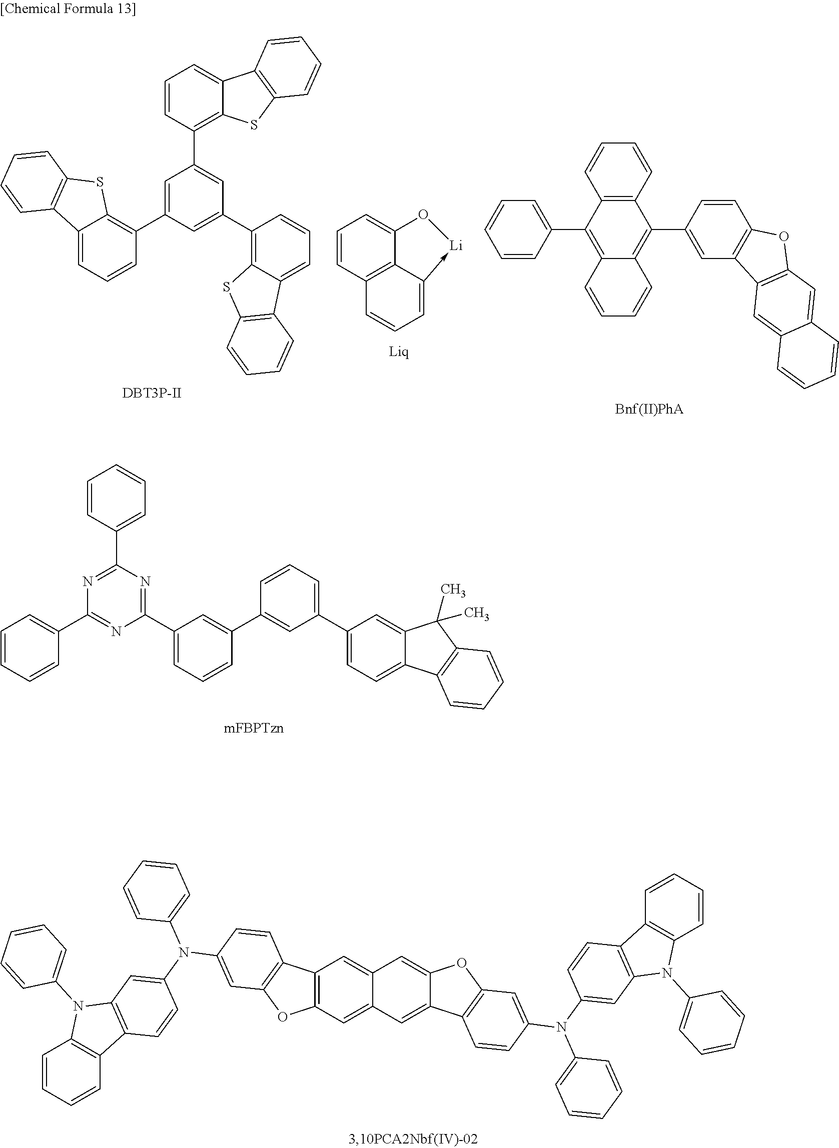

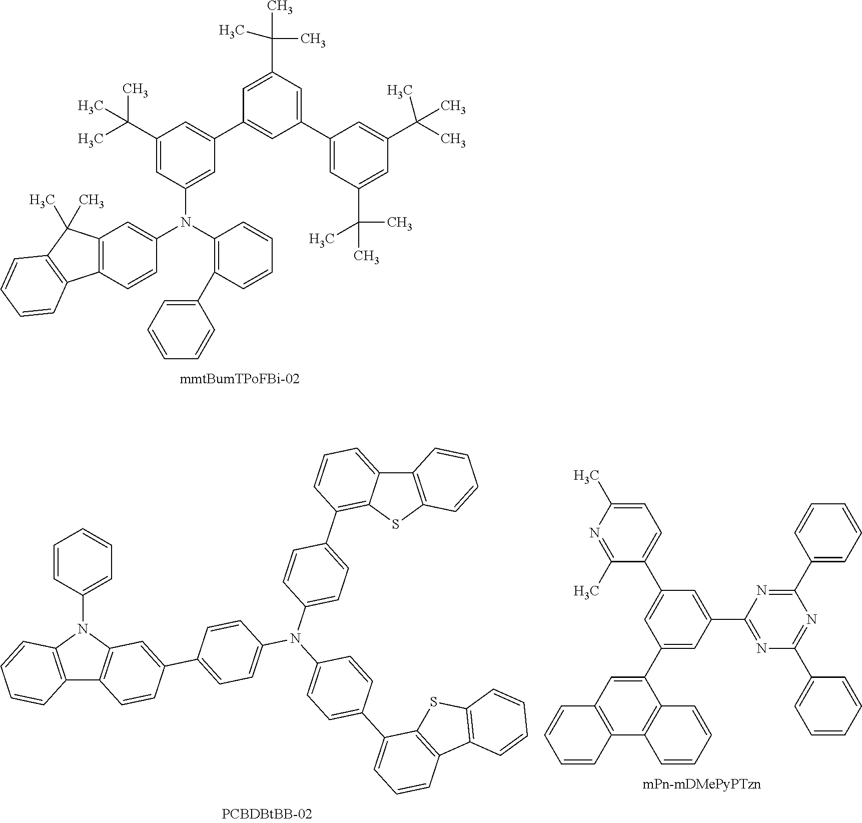

### <<Fabrication Method of Light-Emitting Device 1>>

The light-emitting device 1 described in this example was fabricated using a method including the following steps.

### [First Step]

In a first step, a reflective film REF was formed. Specifically, the reflective film REF was formed by a sputtering method using Ag as a target.

The reflective film REF contains Ag and has a thickness of 100 nm.

### [Second Step]

In a second step, a conductive film TCF was formed over the reflective film REF. Specifically, the conductive film TCF was formed by a sputtering method using indium oxide-tin oxide containing silicon or silicon oxide (abbreviation: ITSO) as a target.

The conductive film TCF contains ITSO and has a thickness of 10 nm and an area of 4 mm² mm×2 mm).

Next, a substrate over which the electrode 101 was formed was washed with water, baked at 200° C. for an hour, and then subjected to UV ozone treatment for 370 seconds. After that, the substrate was transferred into a vacuum evaporation apparatus where the pressure was reduced to approximately 10⁻⁴ Pa, and vacuum baking was performed at 170° C. for 30 minutes in a heating chamber of the vacuum evaporation apparatus. Then, the substrate was cooled down for approximately 30 minutes.

### [Third Step]

In a third step, the layer 104 was formed over the electrode 101. Specifically, materials were co-deposited by a resistance-heating method.

The layer 104 contains 4,4′-bis(dibenzothiophen-4-yl)-4″-(9-phenyl-9H-carbazol-2-yl)triphenylamine (abbreviation: PCBDBtBB-02) and an electron acceptor material (abbreviation: OCHD-001) at PCBDBtBB-02:OCHD-001=1:0.1 (weight ratio), and has a thickness of 10 nm.

### [Fourth Step]

In a fourth step, the layer 112A was formed over the layer 104. Specifically, the material CTM1 was deposited by a resistance-heating method.

The layer 112A contains PCBDBtBB-02 and has a thickness of 70 nm. Moreover, PCBDBtBB-02 has a refractive index of 2.02 with respect to light having a wavelength of 460 nm.

### [Fifth Step]

In a fifth step, the layer 112B was formed over the layer 112A. Specifically, the material CTM2 was deposited by a resistance-heating method.

As the material CTM2, N-(1,1′-biphenyl-2-yl)-N-(3,3″,5′,5″-tetra-t-butyl-1,1′:3′,1″-terphenyl-5-yl)-9,9-dimethyl-9H-fluoren-2-amine (abbreviation: mmtBumTPoFBi-02) was used. The layer 112B contains mmtBumTPoFBi-02 and has a thickness of 35 nm. Moreover, mmtBumTPoFBi-02 has a refractive index of 1.69 with respect to light having a wavelength of 460 nm.

### [Sixth Step]

In a sixth step, a layer 112C was formed over the layer 112B. Specifically, a material was deposited by a resistance-heating method.

The layer 112C contains PCBDBtBB-02 and has a thickness of 10 nm.

### [Seventh Step]

In a seventh step, the layer 111 was formed over the layer 112C. Specifically, materials were co-deposited by a resistance-heating method.

The layer 111 contains 2-(10-phenyl-9-anthracenyl)-benzo[b]naphtho[2,3-d]furan (abbreviation: Bnf(II)PhA) and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) at Bnf(II)PhA:3,10PCA2Nbf(IV)-02=1:0.015 (weight ratio), and has a thickness of 25 nm.

### [Eighth Step]

In an eighth step, the layer 113A was formed over the layer 111. Specifically, a material was deposited by a resistance-heating method.

The layer 113A contains 2-[3′-(9,9-dimethyl-9H-fluoren-2-yl)-1,1′-biphenyl-3-yl]-4,6-diphenyl-1,3,5-triazine (abbreviation: mFBPTzn) and has a thickness of 10 nm.

### [Ninth Step]

In a ninth step, the layer 113B was formed over the layer 113A. Specifically, materials were co-deposited by a resistance-heating method.

The layer 113B contains 2-[3-(2,6-dimethyl-3-pyridinyl)-5-(9-phenanthrenyl)phenyl]-4,6-diphenyl-1,3,5-triazine (abbreviation: mPn-mDMePyPTzn) and 8-hydroxyquinolinato-lithium (abbreviation: Liq) at mPn-mDMePyPTzn:Liq=1:1 (weight ratio) and has a thickness of 20 nm.

### [Tenth Step]

In a tenth step, the layer 105 was formed over the layer 113B. Specifically, a material was deposited by a resistance-heating method.

The layer 105 contains LiF and has a thickness of 1 nm.

### [Eleventh Step]

In an eleventh step, the electrode 102 was formed over the layer 105. Specifically, materials were co-deposited by a resistance-heating method.

The electrode 102 contains Ag and Mg at Ag:Mg=10:1 (volume ratio) and has a thickness of 15 nm.

### [Twelfth Step]

In a twelfth step, a layer CAP was formed over the electrode 102. Specifically, a material was deposited by a resistance-heating method.

The layer CAP contains 4,4′,4″-(benzene-1,3,5-triyl)tri(dibenzothiophene)) (abbreviation: DBT3P-II) and has a thickness of 70 nm.

### <<Operation Characteristics of Light-Emitting Device 1>>

When supplied with electric power, the light-emitting device 1 emitted the light EL1 (see FIG. 15A). Operation characteristics of the light-emitting device 1 were measured (see FIG. 16 to FIG. 21). The measurement was performed at room temperature with a spectroradiometer (UR-UL1R produced by TOPCON TECHNOHOUSE CORPORATION).

Table 2 shows main initial characteristics of the light-emitting device 1 emitting light at a luminance of approximately 1000 cd/m². Note that initial characteristics of the comparative light-emitting device 1 are also shown in Table 2, and its structure will be described later.

TABLE 2

| | | | Current | | | Current | |
|---|---|---|---|---|---|---|---|
| | Voltage | Current | density | Chromaticity | Chromaticity | efficiency | B.I. |
| | (V) | (mA) | (mA/cm²) | x | y | (cd/A) | (cd/A/y) |
| Light-emitting device 1 | 4.0 | 0.41 | 10.2 | 0.14 | 0.05 | 9.1 | 184 |
| Comparative light- | 4.0 | 0.54 | 13.5 | 0.14 | 0.05 | 8.1 | 172 |
| emitting device 1 | | | | | | | |

Note that the blue index (BI) is a value obtained by further dividing current efficiency (cd/A) by chromaticity y, and is one of the indicators representing characteristics of blue light emission. As the chromaticity y is smaller, the color purity of blue light emission tends to be higher. With high color purity for blue light emission, a wide range of blue can be expressed even with a small number of luminance components; hence, using blue light emission with high color purity reduces the luminance needed for expressing blue, leading to lower power consumption. Thus, BI that is based on chromaticity y, which is one of the indicators of color purity of blue, is suitably used as a means for showing efficiency of blue light emission. The light-emitting device with higher BI can be regarded as a blue light-emitting device having more favorable efficiency for a display.

The light-emitting device 1 was found to have favorable characteristics. For example, the light-emitting device 1 obtained luminance equivalent to that of the comparative light-emitting device 1 with a driving voltage equivalent to that of the comparative light-emitting device 1 at a current density lower than that of the comparative light-emitting device 1 (see Table 2). That is, the light-emitting device 1 obtained equivalent luminance with power consumption lower than that of the comparative light-emitting device 1. Moreover, the light-emitting device 1 exhibited higher current efficiency than the comparative light-emitting device 1 (see Table 2 and FIG. 17). The light-emitting device 1 exhibited a blue index that is approximately 1.07 times that of the comparative light-emitting device 1 (see Table 2 and FIG. 20). As a result, a novel light-emitting device that is highly convenient, useful, or reliable was successfully provided.

### Reference Example 1

The fabricated comparative light-emitting device 1 described in this example differs from the light-emitting device 1 in the thickness of the layer 112B and the material CTM2 used for the layer 112B. Specifically, the comparative light-emitting device 1 differs from the light-emitting device 1 in that PCBDBtBB-02 was used as the material CTM2 instead of mmtBumTPoFBi-02. In other words, the same material was used as the material CTM1 and the material CTM2, and the layer 112A, the layer 112B, and the layer 112C were formed as one region.

### <<Fabrication Method of Comparative Light-Emitting Device 1>>

The comparative light-emitting device 1 was fabricated using a method including the following steps.

Note that the fabrication method of the comparative light-emitting device 1 differs from the fabrication method of the light-emitting device 1 in that in the step of forming the layer 112B, PCBDBtBB-02 is used instead of mmtBumTPoFBi-02 and a thickness of 30 nm is used instead of a thickness of 35 nm. In other words, the layer 112A, the layer 112B, and the layer 112C were formed using PCBDBtBB-02 to have a total thickness of 110 nm. Different portions are described in detail here, and the above description is referred to for portions formed by a similar method.

### [Fifth Step]

In the fifth step, the layer 112B was formed over the layer 112A. Specifically, a material was deposited by a resistance-heating method.

The layer 112B contains PCBDBtBB-02 and has a thickness of 30 nm.

Table 2 shows main initial characteristics of the comparative light-emitting device 1.

### Example 2

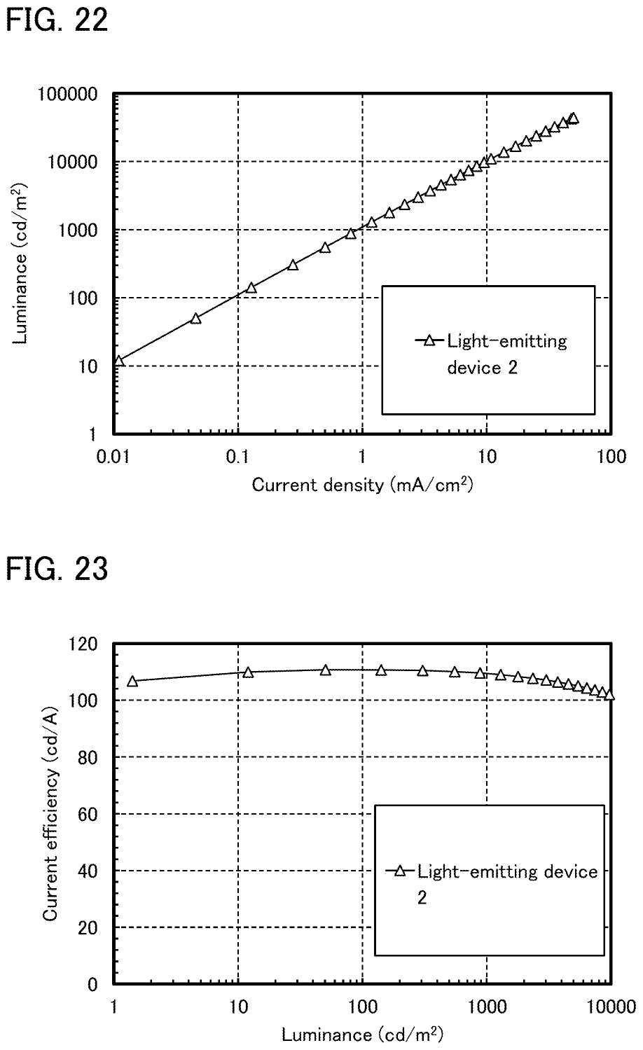

In this example, the fabricated light-emitting device 2 of one embodiment of the present invention is described with reference to FIG. 22 to FIG. 27.

FIG. 22 is a graph showing current density—luminance characteristics of the light-emitting device 2.

FIG. 23 is a graph showing luminance—current efficiency characteristics of the light-emitting device 2.

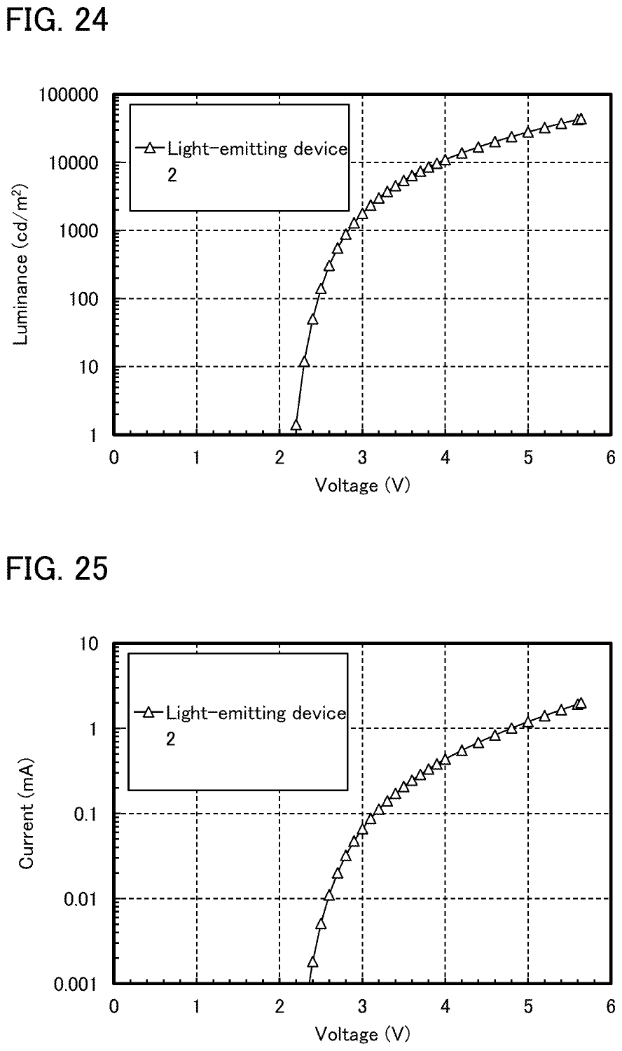

FIG. 24 is a graph showing voltage—luminance characteristics of the light-emitting device 2.

FIG. 25 is a graph showing voltage—current characteristics of the light-emitting device 2.

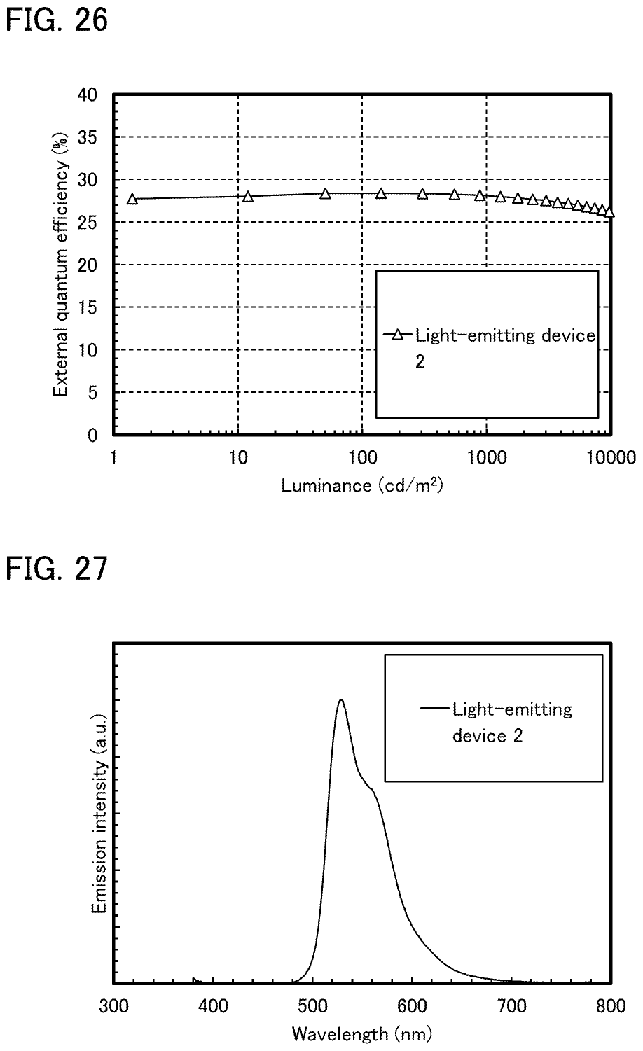

FIG. 26 is a graph showing luminance—external quantum efficiency characteristics of the light-emitting device 2. Note that the external quantum efficiency was calculated from luminance assuming that the light distribution characteristics of the light-emitting device are Lambertian type.

FIG. 27 is a graph showing an emission spectrum of the light-emitting device 2 emitting light at a luminance of 1000 cd/m².

### <Light-Emitting Device 2>

The fabricated light-emitting device 2 described in this example includes a function of emitting the light EL1, the electrode 101, the electrode 102, and the unit 103 (see FIG. 15B).

The light EL1 has the spectrum ϕ1, and the spectrum ϕ1 has a maximum peak at the wavelength λ1 nm.

The electrode 102 includes a region overlapping with the electrode 101. The unit 103 includes a region positioned between the electrode 101 and the electrode 102, and the unit 103 includes the layer 111, the layer 112, and the layer 113.

The layer 111 includes a region positioned between the layer 112 and the layer 113, and the layer 111 contains a light-emitting material.

The layer 112 includes the layer 112A and the layer 112B. The layer 112B includes a region positioned between the layer 112A and the layer 111, and the layer 112B is in contact with the layer 112A.

The layer 112A has a refractive index of 1.86 with respect to light having a wavelength of 530 nm.

The layer 112B has a refractive index of 1.67 with respect to light having a wavelength of 530 nm, and the refractive index 1.67 is lower than the refractive index 1.86.

There is a difference of 0.19 between the refractive index 1.67 and the refractive index 1.86.

In the fabricated light-emitting device 2 described in this example, the layer 111 has a thickness of 40 nm, and the layer 112A has a distance of 40 nm from the layer 111.

When the distance d is 40 nm, the thickness t is 40 nm, the wavelength λ is 530 nm, and the refractive index n2 is 1.67, the value of (d+t/2)×n2 is 100.2 nm. Furthermore, the value of 0.5×0.25×530 nm is 66.25 nm, and the value of 1.5×0.25×530 nm is 198.75 nm. That is, 100.2 nm is in the range of 66.25 nm and 198.75 nm.

In the light-emitting device 2, the layer 112B has a function of inhibiting transport of carriers from the layer 111 toward the layer 112A. Specifically, the layer 112B has a function of inhibiting transport of electrons.

### <<Structure of Light-Emitting Device 2>>

Table 3 shows the structure of the light-emitting device 2. Structural formulae of the materials used in the light-emitting device described in this example are shown below. Note that Ir(ppy)2(mbfpypy-d3) in the table represents Ir(ppy)₂(mbfpypy-d3).

TABLE 3

| | | | | Thick- |
|---|---|---|---|---|
| | Reference | | Composition | ness/ |
| Component | numeral | Material | ratio | nm |
| Electrode | 102 | Al | | 200 |
| Layer | 105 | Liq | | 1 |
| Layer | 113B | mPn-mDMePyPTzn:Liq | 1:1 | 25 |
| Layer | 113A | mFBPTzn | | 10 |
| Layer | 111 | BP-Icz(II)Tzn:PCCP: | 0.5:0.5:0.10 | 40 |
| | | Ir(ppy)2(mbfpypy-d3) | | |
| Layer | 112B | mmtBumTPchPAF-04 | | 40 |
| Layer | 112A | PCBBiF | | 100 |
| Layer | 104 | PCBBiF:OCHD-001 | 1:0.03 | 10 |
| Electrode | 101 | ITSO | | 110 |

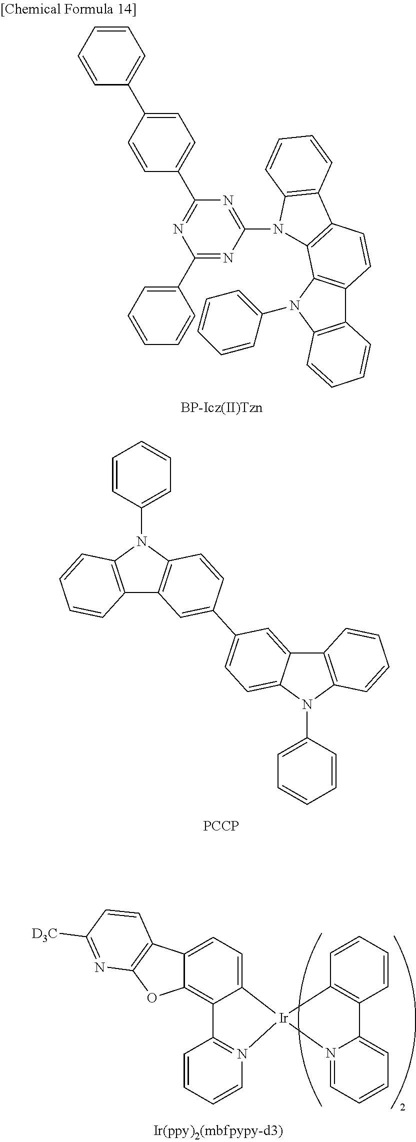

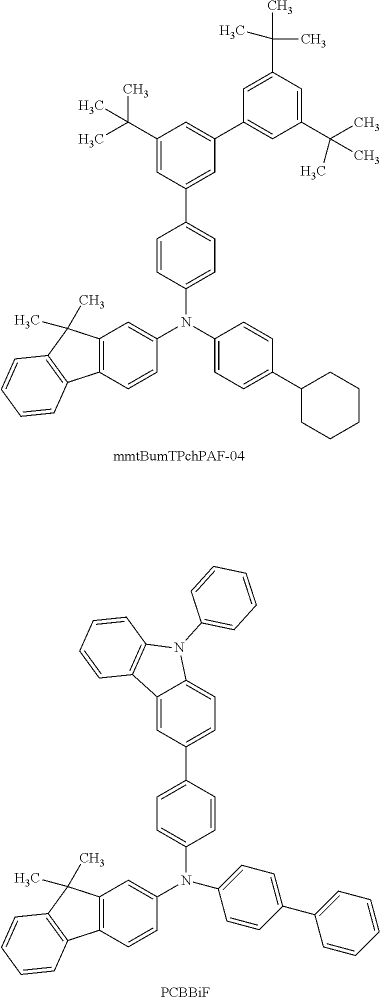

### <<Fabrication Method of Light-Emitting Device 2>>

The light-emitting device 2 described in this example was fabricated using a method including the following steps.

### [First Step]

In a first step, the electrode 101 was formed. Specifically, the electrode 101 was formed by a sputtering method using indium oxide-tin oxide containing silicon or silicon oxide (ITSO) as a target.

The electrode 101 contains ITSO and has a thickness of 110 nm and an area of 4 mm² (2 mm×2 mm).

Next, a substrate over which the electrode 101 was formed was washed with water, baked at 200° C. for an hour, and then subjected to UV ozone treatment for 370 seconds. After that, the substrate was transferred into a vacuum evaporation apparatus where the pressure was reduced to approximately 10⁻⁴ Pa, and vacuum baking was performed at 170° C. for 30 minutes in a heating chamber of the vacuum evaporation apparatus. Then, the substrate was cooled down for approximately 30 minutes.

### [Second Step]

In a second step, the layer 104 was formed over the electrode 101. Specifically, materials were co-deposited by a resistance-heating method.

The layer 104 contains N-(1,1′-biphenyl-4-yl)-9,9-dimethyl-N-[4-(9-phenyl-9H-carbazol-3-yl)phenyl]-9H-fluoren-2-amine (abbreviation: PCBBiF) and OCHD-001 at PCBBiF:OCHD-001=1:0.03 (weight ratio), and has a thickness of 10 nm.

### [Third Step]

In a third step, the layer 112A was formed over the layer 104. Specifically, the material CTM1 was deposited by a resistance-heating method.

The layer 112A contains PCBBiF and has a thickness of 100 nm. In addition, PCBBiF has a refractive index of 1.86 with respect to light having a wavelength of 530 nm.

### [Fourth Step]

In a fourth step, the layer 112B was formed over the layer 112A. Specifically, the material CTM2 was deposited by a resistance-heating method.

The layer 112B contains N-(3″,5′,5″-tri-t-butyl-1,1′:3′,1″-terphenyl-4-yl)-N-(4-cyclohexylphenyl)-9,9-dimethyl-9H-fluoren-2-amine (abbreviation: mmtBumTPchPAF-04) and has a thickness of 40 nm. Moreover, mmtBumTPchPAF-04 has a refractive index of 1.67 with respect to light having a wavelength of 530 nm.

### [Fifth Step]

In a fifth step, the layer 111 was formed over the layer 112B. Specifically, materials were co-deposited by a resistance-heating method.

The layer 111 contains 11-(4-[1,1′-biphenyl]-4-yl-6-phenyl-1,3,5-triazin-2-yl)-11,12-dihydro-12-phenyl-indolo[2,3-a]carbazole (abbreviation: BP-Icz(II)Tzn), 3,3′-bis(9-phenyl-9H-carbazole) (abbreviation: PCCP), and [2-d3-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)) at BP-Icz(II)Tzn:PCCP:Ir(ppy)₂(mbfpypy-d3)=0.5:0.5:0.10 (weight ratio), and has a thickness of 40 nm.

### [Sixth Step]

In a sixth step, the layer 113A was formed over the layer 111. Specifically, a material was deposited by a resistance-heating method.

The layer 113A contains mFBPTzn and has a thickness of 10 nm.

### [Seventh Step]

In a seventh step, the layer 113B was formed over the layer 113A. Specifically, materials were co-deposited by a resistance-heating method.

The layer 113B contains mPn-mDMePyPTzn and Liq at mPn-mDMePyPTzn:Liq=1:1 (weight ratio) and has a thickness of 25 nm.

### [Eighth Step]

In an eighth step, the layer 105 was formed over the layer 113B. Specifically, a material was deposited by a resistance-heating method.

The layer 105 contains Liq and has a thickness of 1 nm.

### [Ninth Step]

In a ninth step, the electrode 102 was formed over the layer 105. Specifically, a material was deposited by a resistance-heating method.

The electrode 102 contains Al and has a thickness of 200 nm.

### <<Operation Characteristics of Light-Emitting Device 2>>

When supplied with electric power, the light-emitting device 2 emitted the light EL1 (see FIG. 15B). Operation characteristics of the light-emitting device 1 were measured (see FIG. 22 to FIG. 27). Note that the measurement was performed at room temperature.

Table 4 shows main initial characteristics of the light-emitting device 2 emitting light at a luminance of approximately 1000 cd/m².

TABLE 4

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 2.8 | 0.03 | 0.8 | 0.34 | 0.63 | 109.6 | 28.1 |
| device 2 | | | | | | | |

The light-emitting device 2 was found to have favorable characteristics. For example, the light-emitting device 2 exhibited extremely high current efficiency (see Table 2 and FIG. 23). Moreover, the light-emitting device 2 exhibited extremely high external quantum efficiency (see Table 2 and FIG. 26). As a result, a novel light-emitting device that is highly convenient, useful, or reliable was successfully provided.

### Synthesis Example 1

In this example, a method of synthesizing the hole-transport material with a low refractive index described in Embodiment 1 is described.

First, a method of synthesizing NA-bis(4-cyclohexylphenyl)-N-(9,9-dimethyl-9H-fluoren-2yl)amine (abbreviation: dchPAF) is described in detail. The structure of dchPAF is shown below.

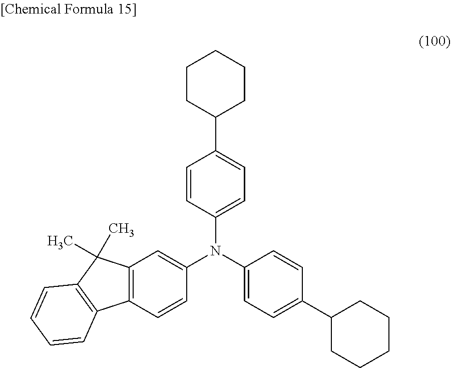 [SMILES: CC1(C)c2ccccc2-c2ccc(N(c3ccc(C4CCCCC4)cc3)c3ccc(C4CCCCC4)cc3)cc21]

### Step 1: Synthesis of N,N-bis(4-cyclohexylphenyl)-N-(9,9-dimethyl-9H-fluoren-2yl)amine (abbreviation: dchPAF)

Into a three-neck flask were put 10.6 g (51 mmol) of 9,9-dimethyl-9H-fluoren-2-amine, 18.2 g (76 mmol) of 4-cyclohexyl-1-bromobenzene, 21.9 g (228 mmol) of sodium-tert-butoxide, and 255 mL of xylene. The mixture was degassed under reduced pressure, and then the air in the flask was replaced with nitrogen. The mixture was stirred while being heated to approximately 50° C. Then, 370 mg (1.0 mmol) of allylpalladium(II) chloride dimer (abbreviation: [(Allyl)PdCl]₂] and 1660 mg (4.0 mmol) of di-tert-butyl(1-methyl-2,2-diphenylcyclopropyl)phosphine (abbreviation: cBRIDP (registered trademark)) were added, and the mixture was heated at 120° C. for approximately 5 hours. After that, the temperature of the flask was lowered to approximately 60° C., and approximately 4 mL of water was added to the mixture, so that a solid was precipitated. The precipitated solid was separated by filtration. The filtrate was concentrated, and the obtained solution was purified by silica gel column chromatography. The obtained solution was concentrated to give a concentrated toluene solution. The toluene solution was dropped into ethanol for reprecipitation. The precipitate was filtrated at approximately 10° C. and the obtained solid was dried at approximately 80° C. under reduced pressure, so that 10.1 g of a target white solid was obtained in a yield of 40%. The synthesis scheme of dchPAF in Step 1 is shown below.

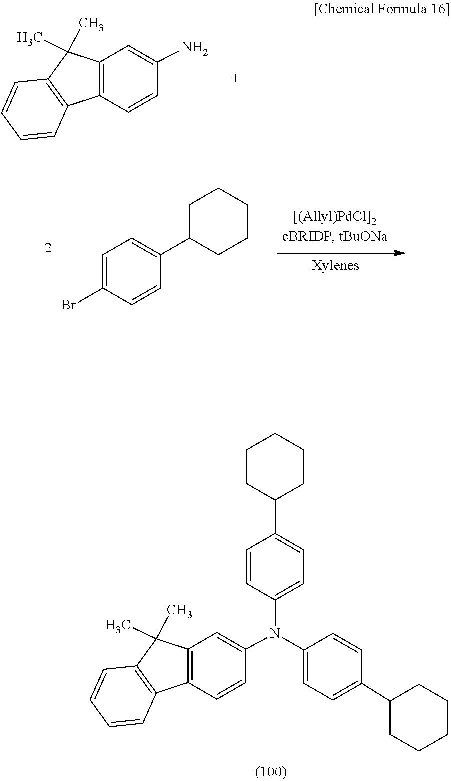 [SMILES: CC1(C)c2ccccc2-c2ccc(N)cc21 ; Brc1ccc(C2CCCCC2)cc1 ; CC1(C)c2ccccc2-c2ccc(N(c3ccc(C4CCCCC4)cc3)c3ccc(C4CCCCC4)cc3)cc21]

Analysis results by nuclear magnetic resonance spectroscopy (1H-NMR) of the white solid obtained in Step 1 are shown below. The results show that dchPAF was synthesized in this synthesis example.

¹H-NMR. δ(CDCl₃): 7.60 (d, 1H, J=7.5 Hz), 7.53 (d, 1H, J=8.0 Hz), 7.37 (d, 2H, J=7.5 Hz), 7.29 (td, 1H, J=7.5 Hz, 1.0 Hz), 7.23 (td, 1H, J=7.5 Hz, 1.0 Hz), 7.19 (d, 1H, J=1.5 Hz), 7.06 (m, 8H), 6.97 (dd, 1H, J=8.0 Hz, 1.5 Hz), 2.41-2.51 (brm, 2H), 1.79-1.95 (m, 8H), 1.70-1.77 (m, 2H), 1.33-1.45 (brm, 14H), 1.19-1.30 (brm, 2H).

Similarly, organic compounds represented by Structural Formula (101) to Structural Formula (105) below were synthesized.

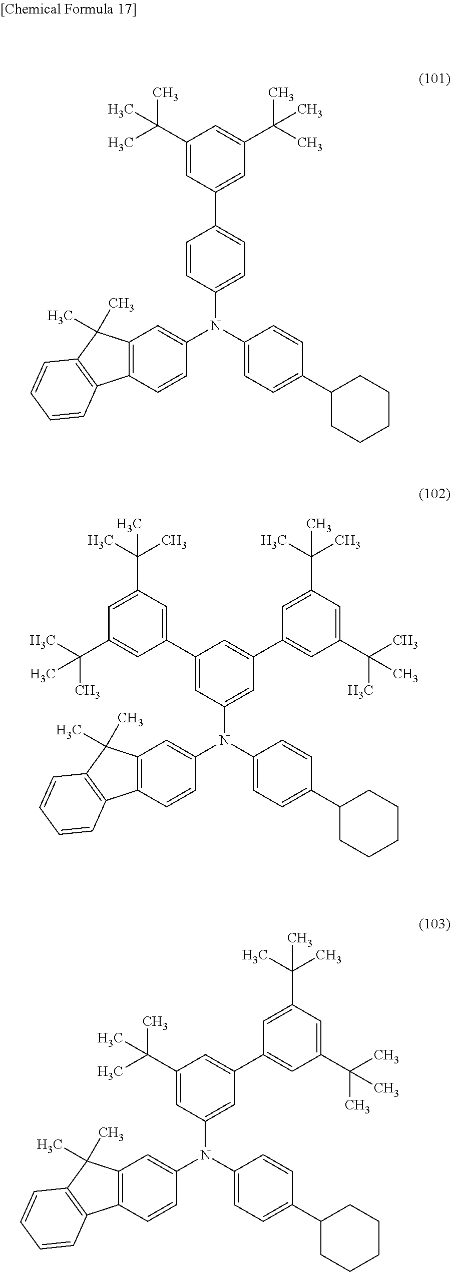 [SMILES: CC(C)(C)c1cc(-c2ccc(N(c3ccc(C4CCCCC4)cc3)c3ccc4c(c3)C(C)(C)c3ccccc3-4)cc2)cc(C(C)(C)C)c1 ; CC(C)(C)c1cc(-c2cc(-c3cc(C(C)(C)C)cc(C(C)(C)C)c3)cc(N(c3ccc(C4CCCCC4)cc3)c3ccc4c(c3)C(C)(C)c3ccccc3-4)c2)cc(C(C)(C)C)c1 ; CC(C)(C)c1cc(-c2cc(C(C)(C)C)cc(C(C)(C)C)c2)cc(N(c2ccc(C3CCCCC3)cc2)c2ccc3c(c2)C(C)(C)c2ccccc2-3)c1]

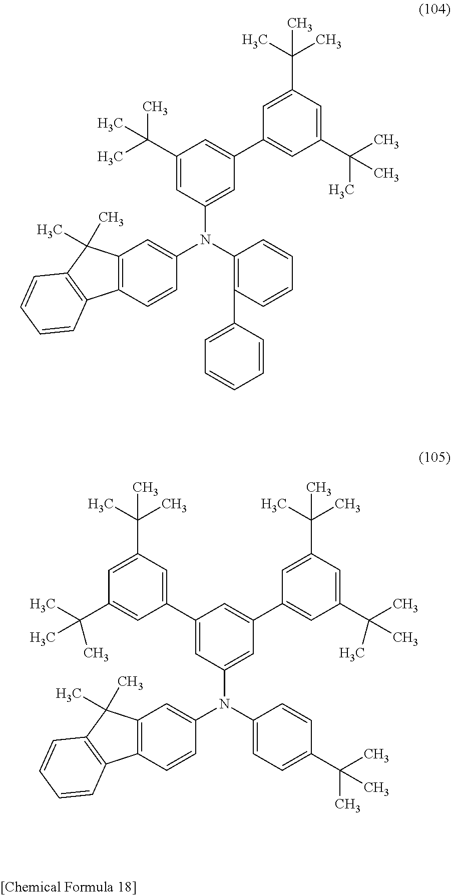 [SMILES: CC(C)(C)c1cc(-c2cc(C(C)(C)C)cc(C(C)(C)C)c2)cc(N(c2ccc3c(c2)C(C)(C)c2ccccc2-3)c2ccccc2-c2ccccc2)c1 ; CC(C)(C)c1ccc(N(c2cc(-c3cc(C(C)(C)C)cc(C(C)(C)C)c3)cc(-c3cc(C(C)(C)C)cc(C(C)(C)C)c3)c2)c2ccc3c(c2)C(C)(C)c2ccccc2-3)cc1]

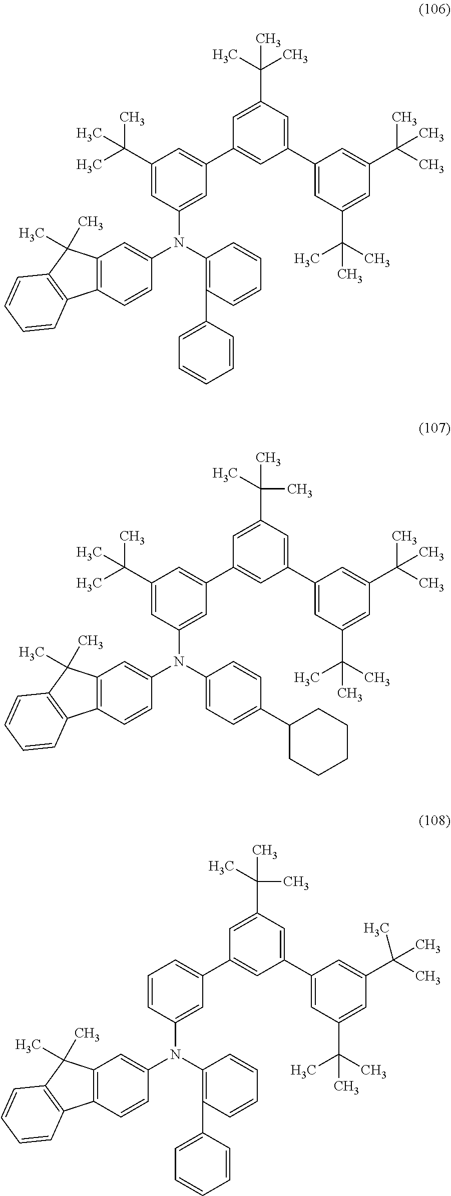 [SMILES: CC(C)(C)c1cc(-c2cc(N(c3ccc4c(c3)C(C)(C)c3ccccc3-4)c3ccccc3-c3ccccc3)cc(C(C)(C)C)c2)cc(-c2cc(C(C)(C)C)cc(C(C)(C)C)c2)c1 ; CC(C)(C)c1cc(-c2cc(N(c3ccc(C4CCCCC4)cc3)c3ccc4c(c3)C(C)(C)c3ccccc3-4)cc(C(C)(C)C)c2)cc(-c2cc(C(C)(C)C)cc(C(C)(C)C)c2)c1 ; CC(C)(C)c1cc(-c2cccc(N(c3ccc4c(c3)C(C)(C)c3ccccc3-4)c3ccccc3-c3ccccc3)c2)cc(-c2cc(C(C)(C)C)cc(C(C)(C)C)c2)c1]

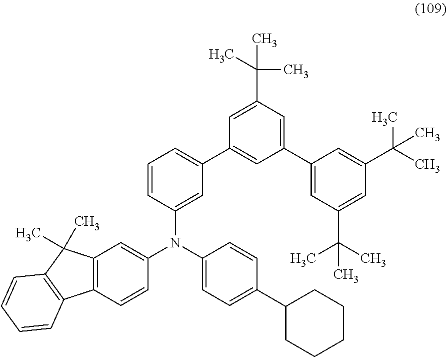 [SMILES: CC(C)(C)c1cc(-c2cccc(N(c3ccc(C4CCCCC4)cc3)c3ccc4c(c3)C(C)(C)c3ccccc3-4)c2)cc(-c2cc(C(C)(C)C)cc(C(C)(C)C)c2)c1]

Analysis results by nuclear magnetic resonance spectroscopy (1H-NMR) of the above organic compounds are shown below.

Structural Formula (101): N-(4-cyclohexylphenyl)-N-(3″,5″-ditertiarybutyl-1,1″-biphenyl-4-yl)-N-(9,9-dimethyl-9H-fluoren-2yl)amine (abbreviation: mmtBuBichPAF) ¹H-NMR. δ(CDCl₃): 7.63 (d, 1H, J=7.5 Hz), 7.57 (d, 1H, J=8.0 Hz), 7.44-7.49 (m, 2H), 7.37-7.42 (m, 4H), 7.31 (td, 1H, J=7.5 Hz, 2.0 Hz), 7.23-7.27 (m, 2H), 7.15-7.19 (m, 2H), 7.08-7.14 (m, 4H), 7.05 (dd, 1H, J=8.0 Hz, 2.0 Hz), 2.43-2.53 (brm, 1H), 1.81-1.96 (m, 4H), 1.75 (d, 1H, J=12.5 Hz), 1.32-1.48 (m, 28H), 1.20-1.31 (brm, 1H).

Structural Formula (102): N-(3,3″,5,5″-tetra-t-butyl-1,1′:3′,1″-terphenyl-5′-yl)-N-(4-cyclohexylphenyl)-9,9-dimethyl-9H-fluoren-2-amine (abbreviation: mmtBumTPchPAF) ¹H-NMR (300 MHz, CDCl₃): δ=7.63 (d, J=6.6 Hz, 1H), 7.58 (d, J=8.1 Hz, 1H), 7.42-7.37 (m, 4H), 7.36-7.09 (m, 14H), 2.55-2.39 (m, 1H), 1.98-1.20 (m, 51H).

Structural Formula (103): N-[(3,3′,5′-t-butyl)-1,1′-biphenyl-5-yl]-N-(4-cyclohexylphenyl)-9,9-dimethyl-9H-fluoren-2-amine (abbreviation: mmtBumBichPAF) ¹H-NMR. δ(CDCl₃): 7.63 (d, 1H, J=7.5 Hz), 7.56 (d, 1H, J=8.5 Hz), 7.37-40 (m, 2H), 7.27-7.32 (m, 4H), 7.22-7.25 (m, 1H), 7.16-7.19 (brm, 2H), 7.08-7.15 (m, 4H), 7.02-7.06 (m, 2H), 2.43-2.51 (brm, 1H), 1.80-1.93 (brm, 4H), 1.71-1.77 (brm, 1H), 1.36-1.46 (brm, 10H), 1.33 (s, 18H), 1.22-1.30 (brm, 10H).

Structural Formula (104): N-(1,1′-biphenyl-2-yl)-N-[(3,3′,5′-tri-t-butyl)-1,1′-biphenyl-5-yl]-9,9-dimethyl-9H-fluoren-2-amine (abbreviation: mmtBumBioFBi) ¹H-NMR. δ(CDCl₃): 7.57 (d, 1H, J=7.5 Hz), 7.40-7.47 (m, 2H), 7.32-7.39 (m, 4H), 7.27-7.31 (m, 2H), 7.27-7.24 (m, 5H), 6.94-7.09 (m, 6H), 6.83 (brs, 2H), 1.33 (s, 18H), 1.32 (s, 6H), 1.20 (s, 9H).

Structural Formula (105): N-(4-tert-butylphenyl)-N-(3,3″,5,5″-tetra-t-butyl-1,1′:3′,1″-terphenyl-5′-yl)-9,9-dimethyl-9H-fluoren-2-amine (abbreviation: mmtBumTPtBuPAF) ¹H-NMR. δ(CDCl₃): 7.64 (d, 1H, J=7.5 Hz), 7.59 (d, 1H, J=8.0 Hz), 7.38-7.43 (m, 4H), 7.29-7.36 (m, 8H), 7.24-7.28 (m, 3H), 7.19 (d, 2H, J=8.5 Hz), 7.13 (dd, 1H, J=1.5 Hz, 8.0 Hz), 1.47 (s, 6H), 1.32 (s, 45H).

Structural Formula (106): N-(1,1′-biphenyl-2-yl)-N-(3,3″,5′,5″-tetra-t-butyl-1,1′:3′,1″-terphenyl-5-yl)-9,9-dimethyl-9H-fluoren-2-amine (abbreviation: mmtBumTPoFBi-02) ¹H-NMR. δ(CDCl₃): 7.56 (d, 1H, J=7.4 Hz), 7.50 (dd, 1H, J=1.7 Hz), 7.33-7.46 (m, 11H), 7.27-7.29 (m, 2H), 7.22 (dd, 1H, J=2.3 Hz), 7.15 (d, 1H, J=6.9 Hz), 6.98-7.07 (m, 7H), 6.93 (s, 1H), 6.84 (d, 1H, J=6.3 Hz), 1.38 (s, 9H), 1.37 (s, 18H), 1.31 (s, 6H), 1.20 (s, 9H).

Structural Formula (107): N-(4-cyclohexylphenyl)-N-(3,3″,5′,5″-tetra-t-butyl-1,1′:3′,1″-terphenyl-5-yl)-9,9-dimethyl-9H-fluoren-2-amine (abbreviation: mmtBumTPchPAF-02) ¹H-NMR. δ(CDCl₃): 7.62 (d, 1H, J=7.5 Hz), 7.56 (d, 1H, J=8.0 Hz), 7.50 (dd, 1H, J=1.7 Hz), 7.46-7.47 (m, 2H), 7.43 (dd, 1H, J=1.7 Hz), 7.37-7.39 (m, 3H), 7.29-7.32 (m, 2H), 7.23-7.25 (m, 2H), 7.20 (dd, 1H, J=1.7 Hz), 7.09-7.14 (m, 5H), 7.05 (dd, 1H, J=2.3 Hz), 2.46 (brm, 1H), 1.83-1.88 (m, 4H), 1.73-1.75 (brm, 1H), 1.42 (s, 6H), 1.38 (s, 9H), 1.36 (s, 18H), 1.29 (s, 9H).

Structural Formula (108): N-(1,1′-biphenyl-2-yl)-N-(3″,5′,5″-tri-t-butyl-1,1′:3′,1″-terphenyl-5-yl)-9,9-dimethyl-9H-fluoren-2-amine (abbreviation: mmtBumTPoFBi-03) ¹H-NMR. δ(CDCl₃): 7.55 (d, 1H, J=7.4 Hz), 7.50 (dd, 1H, J=1.7 Hz), 7.42-7.43 (m, 3H), 7.27-7.39 (m, 10H), 7.18-7.25 (m, 4H), 7.00-7.12 (m, 4H), 6.97 (dd, 1H, J=6.3 Hz, 1.7 Hz), 6.93 (d, 1H, J=1.7 Hz), 6.82 (dd, 1H, J=7.3 Hz, 2.3 Hz), 1.37 (s, 9H), 1.36 (s, 18H), 1.29 (s, 6H).

Structural Formula (109): N-(4-cyclohexylphenyl)-N-(3″,5′,5″-tri-t-butyl-1,1′:3′,1″-terphenyl-5-yl)-9,9-dimethyl-9H-fluoren-2-amine (abbreviation: mmtBumTPchPAF-03) ¹H-NMR. δ(CDCl₃): 7.62 (d, 1H, J=7.5 Hz), 7.56 (d, 1H, J=8.6 Hz), 7.51 (dd, 1H, J=1.7 Hz), 7.48 (dd, 1H, J=1.7 Hz), 7.46 (dd, 1H, J=1.7 Hz), 7.42 (dd, 1H, J=1.7 Hz), 7.37-7.39 (m, 4H), 7.27-7.33 (m, 2H), 7.23-7.25 (m, 2H), 7.05-7.13 (m, 7H), 2.46 (brm, 1H), 1.83-1.90 (m, 4H), 1.73-1.75 (brm, 1H), 1.41 (s, 6H), 1.37 (s, 9H), 1.35 (s, 18H).

The substances described above each have an ordinary refractive index higher than or equal to 1.50 and lower than or equal to 1.75 in a blue light emission range (455 nm to 465 nm) or an ordinary refractive index higher than or equal to 1.45 and lower than or equal to 1.70 with respect to 633-nm light, which is usually used for measurement of refractive indices.

### Synthesis Example 2

In this example, a method of synthesizing the hole-transport material with a low refractive index described in Embodiment 1 is described.

A method of synthesizing N-(4-cyclohexylphenyl)-N-(3″,5′,5″-tri-t-butyl-1,1′:3′,1″-terphenyl-4-yl)-9,9-dimethyl-9H-fluoren-2-amine (abbreviation: mmtBumTPchPAF-04) is described. The structure of mmtBumTPchPAF-04 is shown below.

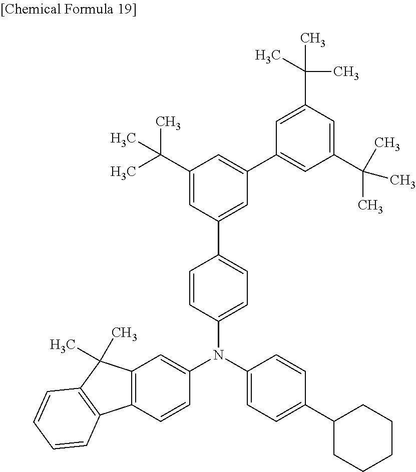 [SMILES: CC(C)(C)c1cc(-c2ccc(N(c3ccc(C4CCCCC4)cc3)c3ccc4c(c3)C(C)(C)c3ccccc3-4)cc2)cc(-c2cc(C(C)(C)C)cc(C(C)(C)C)c2)c1]

### Step 1: Synthesis of 4-bromo-3″,5′,5″-tri-tert-butyl-1,1′:3′,1″-terphenyl

In a three-neck flask were put 9.0 g (20.1 mmol) of 2-(3′,5,5′-tri-tert-butyl[1,1′-biphenyl]-3-yl)-4,4,5,5-tetramethyl-1,3,2-dioxaborolane, 6.8 g (24.1 mmol) of 1-bromo-4-iodobenzene, 8.3 g (60.3 mmol) of potassium carbonate, 100 mL of toluene, 40 mL of ethanol, and 30 mL of tap water. The mixture was degassed under reduced pressure, and then the air in the flask was replaced with nitrogen. Then, 91 mg (0.40 mmol) of palladium acetate and 211 mg (0.80 mmol) of triphenylphosphine were added, and the mixture was heated at 80° C. for approximately 4 hours. After that, the temperature was lowered to room temperature, and the mixture was separated into an organic layer and an aqueous layer. Magnesium sulfate was added to this solution to eliminate moisture, whereby this solution was concentrated. The obtained hexane solution was purified by silica gel column chromatography, whereby 6.0 g of a target white solid was obtained in a yield of 62.5%. The synthesis scheme of 4-bromo-3″,5′,5″-tri-tert-butyl-1,1′:3′,1″-terphenyl in Step 1 is shown below.

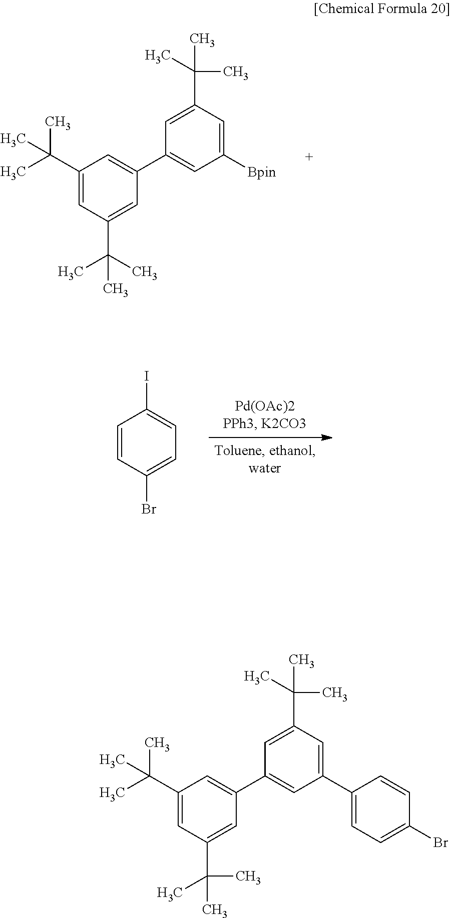

### Step 2: Synthesis of mmtBumTPchPAF-04

In a three-neck flask were put 3.0 g (6.3 mmol) of 4-bromo-3″,5′,5″-tri-tert-butyl-1,1′:3′,1″-terphenyl obtained in Step 1, 2.3 g (6.3 mmol) of N-(4-cyclohexylphenyl)-N-(9,9-dimethyl-9H-fluoren-2yl)amine, 1.8 g (18.9 mmol) of sodium tert butoxide, and 32 mL of toluene. The mixture was degassed under reduced pressure, the air in the flask was replaced with nitrogen, 72 mg (0.13 mmol) of bis(dibenzylideneacetone)palladium(0) and 76 mg (0.38 mmol) of tri-tert-butylphosphine were added, and the mixture was heated at 80° C. for approximately 2 hours. After that, the temperature of the flask was lowered to approximately 60° C., approximately 1 mL of water was added, a precipitated solid was separated by filtration, and the solid was washed with toluene. The filtrate was concentrated, and the obtained toluene solution was purified by silica gel column chromatography. The obtained solution was concentrated to give a concentrated toluene solution. Ethanol was added to this toluene solution and the toluene solution was concentrated under reduced pressure, whereby an ethanol suspension was obtained. The solid precipitated in this ethanol suspension was filtrated at approximately 20° C., and the obtained solid was dried at approximately 80° C. under reduced pressure, whereby 4.1 g of a target white solid was obtained in a yield of 85%. The synthesis scheme of mmtBumTPchPAF-04 in Step 2 is shown below.

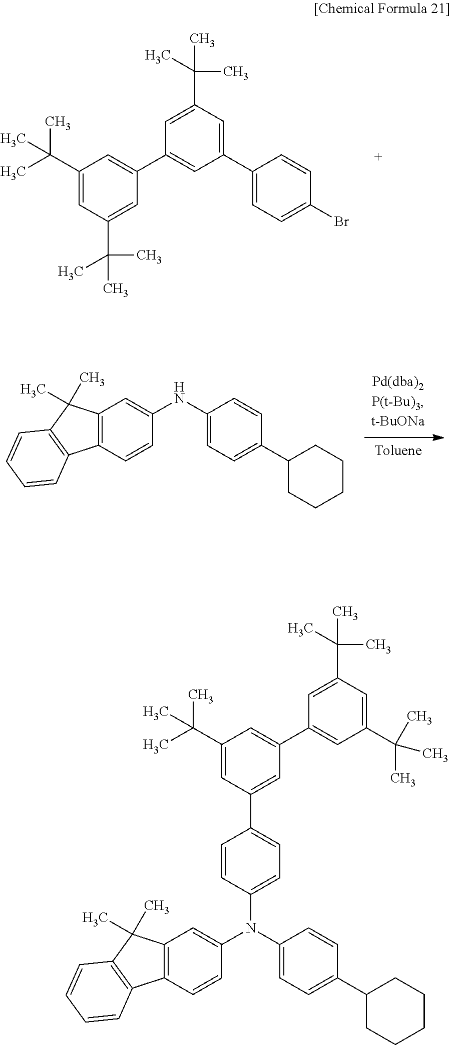 [SMILES: CC(C)(C)c1cc(-c2ccc(Br)cc2)cc(-c2cc(C(C)(C)C)cc(C(C)(C)C)c2)c1 ; CC1(C)c2ccccc2-c2ccc(Nc3ccc(C4CCCCC4)cc3)cc21 ; CC(C)(C)c1cc(-c2ccc(N(c3ccc(C4CCCCC4)cc3)c3ccc4c(c3)C(C)(C)c3ccccc3-4)cc2)cc(-c2cc(C(C)(C)C)cc(C(C)(C)C)c2)c1]

Note that the results of analysis by nuclear magnetic resonance (¹H-NMR) spectroscopy of the white solid obtained in Step 2 above are shown below. The results show that mmtBumTPchPAF-04 was synthesized in this synthesis example.

¹H-NMR. δ(CDCl₃): 7.63 (d, 1H, J=7.5 Hz), 7.52-7.59 (m, 7H), 7.44-7.45 (m, 4H), 7.39 (d, 1H, J=7.4 Hz), 7.31 (dd, 1H, J=7.4 Hz), 7.19 (d, 2H, J=6.6 Hz), 7.12 (m, 4H), 7.07 (d, 1H, J=9.7 Hz), 2.48 (brm, 1H), 1.84-1.93 (brm, 4H), 1.74-1.76 (brm, 1H), 1.43 (s, 18H), 1.39 (brm, 19H), 1.24-1.30 (brm, 1H).

### REFERENCE NUMERALS

CAP: layer, 101: electrode, 102: electrode, 103: unit, 104: layer, 105: layer, 106: intermediate layer, 106A: layer, 106B: layer, 111: layer, 112: layer, 112A: layer, 112B: layer, 112C: layer, 113: layer, 113A: layer, 113B: layer, 150: light-emitting device, 400: substrate, 401: electrode, 403: EL layer, 404: electrode, 405: sealant, 406: sealant, 407: sealing substrate, 412: pad, 420: IC chip, 601: source line driver circuit, 602: pixel portion, 603: gate line driver circuit, 604: sealing substrate, 605: sealant, 607: space, 608: wiring, 610: element substrate, 611: switching FET, 612: current control FET, 613: electrode, 614: insulator, 616: EL layer, 617: electrode, 618: light-emitting device, 623: FET, 700: light-emitting panel, 951: substrate, 952: electrode, 953: insulating layer, 954: partition layer, 955: EL layer, 956: electrode, 1001: substrate, 1002: base insulating film, 1003: gate insulating film, 1006: gate electrode, 1007: gate electrode, 1008: gate electrode, 1020: interlayer insulating film, 1021: interlayer insulating film, 1022: electrode, 1024B: electrode, 1024G: electrode, 1024R: electrode, 1024W: electrode, 1025: partition, 1028: EL layer, 1029: electrode, 1031: sealing substrate, 1032: sealant, 1033: base material, 1034B: coloring layer, 1034G: coloring layer, 1034R: coloring layer, 1035: black matrix, 1036: overcoat layer, 1037: interlayer insulating film, 1040: pixel portion, 1041: driver circuit portion, 1042: peripheral portion, 2001: housing, 2002: light source, 2100: robot, 2101: illuminance sensor, 2102: microphone, 2103: upper camera, 2104: speaker, 2105: display, 2106: lower camera, 2107: obstacle sensor, 2108: moving mechanism, 2110: arithmetic device, 3001: lighting device, 5000: housing, 5001: display portion, 5002: display portion, 5003: speaker, 5004: LED lamp, 5006: connection terminal, 5007: sensor, 5008: microphone, 5012: support, 5013: earphone, 5100: cleaning robot, 5101: display, 5102: camera, 5103: brush, 5104: operation button, 5120: dust, 5140: portable electronic device, 5200: display region, 5201: display region, 5202: display region, 5203: display region, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7210: display portion, 7401: housing, 7402: display portion, 7403: operation button, 7404: external connection port, 7405: speaker, 7406: microphone, 9310: portable information terminal, 9311: functional panel, 9313: hinge, 9315: housing

1. (canceled)

2. A light-emitting device comprising:
a first electrode;
a second electrode;
a first layer;
a second layer; and
a third layer,
wherein the light-emitting device is configured to emit light comprising a first spectrum,
wherein the first spectrum has a maximum peak at a wavelength λ1,
wherein the second electrode comprises a region overlapping with the first electrode,
wherein the first layer comprises a region positioned between the first electrode and the second electrode,
wherein the first layer comprises a region positioned between the second layer and the third layer,
wherein the first layer comprises a light-emitting material,
wherein the second layer comprises a region positioned between the first electrode and the first layer,
wherein the second layer comprises a fourth layer and a fifth layer,
wherein the fifth layer comprises a region positioned between the fourth layer and the first layer,
wherein the fourth layer comprises a first organic compound,
wherein the first organic compound has a first refractive index n1 with respect to light having the wavelength λ1,
wherein the fifth layer is in contact with the fourth layer,
wherein the fifth layer comprises a second organic compound,
wherein the second organic compound has a second refractive index n2 with respect to light having the wavelength λ1, and
wherein the second refractive index n2 is lower than the first refractive index n1.

3. The light-emitting device according to claim 2,
wherein the first refractive index n1 differs from the second refractive index n2 by 0.1 or more and 1.0 or less.

4. The light-emitting device according to claim 2,
wherein the second refractive index n2 is higher than or equal to 1.4 and lower than or equal to 1.75.

5. A light-emitting device comprising:
a first electrode;
a second electrode;
a first layer;
a second layer; and
a third layer,
wherein the second electrode comprises a region overlapping with the first electrode,
wherein the first layer comprises a region positioned between the first electrode and the second electrode,
wherein the first layer comprises a region positioned between the second layer and the third layer,
wherein the first layer comprises a light-emitting material,
wherein the first layer emits photoluminescent light,
wherein the photoluminescent light comprises a second spectrum,
wherein the second spectrum has a maximum peak at a wavelength λ2,
wherein the second layer comprises a region positioned between the first electrode and the first layer,
wherein the second layer comprises a fourth layer and a fifth layer,
wherein the fifth layer comprises a region positioned between the fourth layer and the first layer,
wherein the fourth layer comprises a first organic compound,
wherein the first organic compound has a first refractive index n1 with respect to light having the wavelength λ2,
wherein the fifth layer is in contact with the fourth layer,
wherein the fifth layer comprises a second organic compound,
wherein the second organic compound has a second refractive index n2 with respect to light having the wavelength λ2, and
wherein the second refractive index n2 is lower than the first refractive index n1.

6. The light-emitting device according to claim 5,
wherein the first refractive index n1 differs from the second refractive index n2 by 0.1 or more and 1.0 or less.

7. The light-emitting device comprising: according to claim 5,
wherein the second refractive index n2 is higher than or equal to 1.4 and lower than or equal to 1.75.

8. A light-emitting device comprising:
a first electrode;
a second electrode;
a first layer;
a second layer; and
a third layer,
wherein the second electrode comprises a region overlapping with the first electrode,
wherein the first layer comprises a region positioned between the first electrode and the second electrode,
wherein the first layer comprises a region positioned between the second layer and the third layer,
wherein the first layer comprises a light-emitting material,
wherein the light-emitting material emits photoluminescent light,
wherein the photoluminescent light comprises a third spectrum,
wherein the third spectrum has a maximum peak at a wavelength λ3,
wherein the second layer comprises a region positioned between the first electrode and the first layer,
wherein the second layer comprises a fourth layer and a fifth layer,
wherein the fifth layer comprises a region positioned between the fourth layer and the first layer,
wherein the fourth layer comprises a first organic compound,
wherein the first organic compound has a first refractive index n1 with respect to light having the wavelength λ3,
wherein the fifth layer is in contact with the fourth layer,
wherein the fifth layer comprises a second organic compound,
wherein the second organic compound has a second refractive index n2 with respect to light having the wavelength λ3, and
wherein the second refractive index n2 is lower than the first refractive index n1.

9. The light-emitting device according to claim 8,
wherein the first refractive index n1 differs from the second refractive index n2 by 0.1 or more and 1.0 or less.

10. The light-emitting device according to claim 2,
wherein the fourth layer has a distance d between the fourth layer and the first layer, and
wherein the distance d is greater than or equal to 20 nm and less than or equal to 120 nm.

11. The light-emitting device according to claim 2,
wherein the fourth layer has a distance d between the fourth layer and the first layer,
wherein the first layer has a thickness t, and
wherein the distance d is in a range represented by the thickness t, the wavelength λ1, the second refractive index n2, and the following Formula (1).
$$[ Formula 1 ]$$
$$0.5 \times 0.25 \times λ 1 \leq ( d + \frac{t}{2} ) \times n 2 \leq 1.5 \times 0.25 \times λ 1 \qquad ( 1 )$$

12. The light-emitting device according to claim 2,
wherein the fifth layer is in contact with the first layer, and
wherein the fifth layer is configured to inhibit transport of carriers from the first layer toward the fourth layer.

13. The light-emitting device according to claim 12,
wherein the second organic compound has a hole-transport property,
wherein the second organic compound has a first LUMO level,
wherein the first layer comprises a host material,
wherein the host material has a second LUMO level, and
wherein the second LUMO level is lower than the first LUMO level.

14. The light-emitting device according to claim 2,
wherein the second organic compound is an amine compound.

15. The light-emitting device according to claim 2,
wherein the first organic compound is an amine compound.

16. The light-emitting device according to claim 2,
wherein the second organic compound is a monoamine compound,
wherein the monoamine compound comprises a group of aromatic groups and a nitrogen atom,
wherein the group of aromatic groups comprises a first aromatic group, a second aromatic group, and a third aromatic group,
wherein the nitrogen atom is bonded to the first aromatic group, the second aromatic group, and the third aromatic group,
wherein the group of aromatic groups comprises a substituent,
wherein the substituent comprises sp3 carbon,
wherein the sp3 carbon forms a bond with another atom by an sp3 hybrid orbital, and
wherein the sp3 carbon accounts for higher than or equal to 23% and lower than or equal to 55% of all carbon included in the monoamine compound.

17. A light-emitting apparatus comprising:
the light-emitting device according to claim 2; and a transistor or a substrate.

18. A display apparatus comprising:
the light-emitting device according to claim 2; and a transistor or a substrate.

19. A lighting device comprising:
the light-emitting apparatus according to claim 17; and a housing.

20. An electronic device comprising:
the display apparatus according to claim 18; and a sensor, an operation button, a speaker, or a microphone.

21. The light-emitting device according to claim 8,
wherein the second refractive index n2 is higher than or equal to 1.4 and lower than or equal to 1.75.
